SEQUENCE LISTING (37 sequences)

SEQ ID NO: 1 - protein, 10 aa, Bos taurus
FKDLGEEHFK

SEQ ID NO: 2 - protein, 8 aa, Bos taurus
DLGEEHFK

SEQ ID NO: 3 - protein, 13 aa, Bos taurus
TCVADESHAGCEK

SEQ ID NO: 4 - protein, 12 aa, Bos taurus
SLHTLFGDELCK

SEQ ID NO: 5 - protein, 21 aa, Bos taurus
QEPERNECFLSHKDDSPDLPK

SEQ ID NO: 6 - protein, 16 aa, Bos taurus
NECFLSHKDDSPOLPK

SEQ ID NO: 7 - protein, 19 aa, Bos taurus
ECCHGOLLECADDRADLAK

SEQ ID NO: 8 - protein, 22 aa, Bos taurus
VHKECCHGDLLECADDRADLAK

SEQ ID NO: 9 - protein, 9 aa, Bos taurus
SHCIAEVEK

SEQ ID NO: 10 - protein, 31 aa, Bos taurus
SHCIAEVEKDAIPENLPPLTADFAEDKDVCK

SEQ ID NO: 11 - protein, 13 aa, Bos taurus
DOPHACYSTVFDK

SEQ ID NO: 12 - protein, 25 aa, Bos taurus
EYEATLEECCAKDDPHACYSTVFDK

SEQ ID NO: 13 - protein, 13 aa, Bos taurus
LKHLVDEPONLIK

SEQ ID NO: 14 - protein, 11 aa, Bos taurus
HLVDEPONLIK

SEQ ID NO: 15 - protein, 16 aa, Bos taurus
LFTFHADICTLPOTEK

SEQ ID NO: 16 - protein, 13 aa, Bos taurus
LKHLVDEPQNLIK

SEQ ID NO: 17 - protein, 16 aa, Bos taurus
LFTFHADICTLPDTEK

SEQ ID NO: 18 - protein, 9 aa, Bos taurus
CELAAAMKR

SEQ ID NO: 19 - protein, 16 aa, Bos taurus
NTDGSTDYGILQINSR

SEQ ID NO: 20 - protein, 15 aa, Bos taurus
IVSDGNGMNAWVAWR

SEQ ID NO: 21 - protein, 9 aa, Bos taurus
GTDVQAWIR

SEQ ID NO: 22 - protein, 16 aa, Bos taurus
SEIAHRFKDLGEEHFK

SEQ ID NO: 23 - protein, 10 aa, Bos taurus
FKDLGEEFHK

SEQ ID NO: 24 - protein, 21 aa, Bos taurus
GLVLIAFSQYLQQCPFDEHVK

SEQ ID NO: 25 - protein, 10 aa, Bos taurus
LVNELTEFAK

SEQ ID NO: 26 - protein, 12 aa, Bos taurus
ETYGDMADCCEK

SEQ ID NO: 27 - protein, 15 aa, Bos taurus
AEFVEVTKLVTDLTK

SEQ ID NO: 28 - protein, 19 aa, Bos taurus
ECCHGDLLECADDRADLAK

SEQ ID NO: 29 - protein, 9 aa, Bos taurus
SCHIAEVEK

SEQ ID NO: 30 - protein, 31 aa, Bos taurus
SCHIAEVEKDAIPENLPPLTADFAEDKDVCK

SEQ ID NO: 31 - protein, 13 aa, Bos taurus
DAFLGSFLYEYSR

SEQ ID NO: 32 - protein, 13 aa, Bos taurus
DDPHACYSTVFDK

SEQ ID NO: 33 - protein, 26 aa, Bos taurus
DDPHACYSTVFDKHKLHVDEPQNLIK

SEQ ID NO: 34 - protein, 11 aa, Bos taurus
HLVDEPQNLIK

SEQ ID NO: 35 - protein, 14 aa, Bos taurus
MPCTEDYLSLILNR

SEQ ID NO: 36 - protein, 21 aa, Bos taurus
AFDEKLFTFHADICTLPDTEK

SEQ ID NO: 37 - protein, 16 aa, Bos taurus
LFTFHADICTLPDTEK

(19) United States
(12) Patent Application Publication
(10) Pub. No.: US 2025/0059228 A1
(43) Pub. Date: Feb. 20, 2025

(54) FAST, EASY, AND DIRECT AZIDYLATION OF BIOMOLECULES IN SOLUTION

(71) Applicant: Wisconsin Alumni Research Foundation, Madison, WI (US)

(72) Inventors: Michael Sussman, Cross Plains, WI (US); Benjamin Minkoff, Madison, WI (US); Jamison Wolfer, Cross Plains, WI (US); Heather Burch, Lone Rock, WI (US)

(73) Assignee: Wisconsin Alumni Research Foundation, Madison, WI (US)

(21) Appl. No.: 18/723,010

(22) PCT Filed: Dec. 22, 2022
(86) PCT No.: PCT/US2022/053757

§ 371 Date: Jun. 21, 2024

Related U.S. Application Data
(60) Provisional application No. 63/429,703, filed on Dec. 2, 2022.
(60) Provisional application No. 63/350,462, filed on Jun. 9, 2022.
(60) Provisional application No. 63/292,944, filed on Dec. 22, 2021.

(51) Int. Cl. C07K 1/107; B01J 23/72; C07H 1/00; C07K 1/13; C07K 14/765

(52) U.S. Cl. CPC C07K 1/1077; B01J 23/72; C07H 1/00; C07K 1/13; C07K 14/765

(57) ABSTRACT

A method of attaching an azide moiety to a biomolecule. The method entails contacting a biomolecule in a solution with an azide and an oxidizing agent, for a time and at a temperature wherein at least one azide moiety is covalently bonded to the biomolecule to yield an azidylated biomolecule. The method can be used to identify hydrophobic microenvironments in soluble proteins. Also, a method of attaching a reagent comprising an alkyne to a biomolecule. The method comprises reacting a biomolecule in a solution with an azide and a reagent comprising an alkyne, for a time and at a temperature wherein at least one reagent comprising an alkyne is covalently bonded to the biomolecule via triazole linkage.

### CROSS-REFERENCE TO RELATED APPLICATIONS

Priority is hereby claimed to PCT Application Ser. No. PCT/US2022/053757, filed Dec. 22, 2022, which claims priority to provisional application Ser. No. 63/429,703, filed Dec. 2, 2022, provisional application Ser. No. 63/350,462, filed Jun. 9, 2022, and provisional application Ser. No. 63/292,944, filed Dec. 22, 2021, the content of all of which are incorporated herein by reference.

### FEDERAL FUNDING STATEMENT

This invention was made with government support under HDTRA1-16-1-0049 awarded by the DOD/DTRA and under 1943816, 2010789 and 1546742 awarded by the National Science Foundation and under DE-FG02-88ER13938 awarded by the US Department of Energy. The government has certain rights in the invention.

### SEQUENCE LISTING

The instant application contains a Sequence Listing which has been submitted in an XML file with the USPTO through Patent Center and is hereby incorporated by reference in its entirety. The Sequence Listing XML, created on Apr. 10, 2024, is named “Fast, Easy, and Direct Azidylation of Biomolecules in Solution.xml” and is 33,102 bytes in size.

### BACKGROUND

The development of so-called “click chemistry,” i.e., copper-catalyzed azide-alkyne cycloaddition (“CuAAC”), in the early 2000's opened a new era in the study of molecular interactions. See, for example, Baskin J M, Bertozzi C R. (2007) “Bioorthogonal Click Chemistry: Covalent Labeling in Living Systems,” QSAR Comb Sci. 26:1211-1219. Click chemistry provides an easy way to covalently link molecules together. An early description of the CuAAC reaction is found in Kolb H C, Finn M G, Sharpless K B (2001) “Click Chemistry: Diverse Chemical Function from a Few Good Reactions,” Angewandte Chemie International Edition 40 (11): 2004-2021. Many discoveries, tools, and products have been developed from that initial discovery. Covalently attaching molecules together with click chemistry, however, requires first synthesizing or attaching the reactive azide and alkyne moieties to the molecules that are to be linked or “clicked.” This has proven problematic when working with macromolecular biomolecules such as whole proteins, macromolecular nucleic acids, and the like.

Click chemistry has proven to be well-suited to biomolecular investigations. See, for example, Presolski, Hong, and Finn (2011) “Copper-Catalyzed Azide-Alkyne Click Chemistry for Bioconjugation,” Curr Protoc Chem Biol. 3 (4): 153-162. As noted there, azides and alkynes are small and unobtrusive moieties. They lack the ability to engage in strong hydrogen bonding, as well as acid-base, hydrophobic, coulombic, dipolar, and x-stacking interactions. As a result, they minimally perturb the biological molecules to which they are attached (if at all). The literature now includes a growing number of examples in which azide- or alkyne-derivatized nutrients or cofactors are taken up and incorporated into biological molecules by living cells. By way of a very small sampling, see Kiick K L, Saxon E, Tirrell D A, Bertozzi C R (2002) “Incorporation of Azides into Recombinant Proteins for Chemoselective Modification by the Staudinger Ligation,” Proc Natl Acad Sci USA. 99 (1): 19-24; Ning X, Guo J, Wolfert M A, Boons G J (2008) “Visualizing Metabolically Labeled Glycoconjugates of Living Cells by Copper-Free and Fast Huisgen Cycloadditions,” Angew Chem Int Ed Engl. 47 (12): 2253-5; and Rangan K J, Yang Y Y, Charron G, Hang H C (2010) “Rapid visualization and large-scale profiling of bacterial lipoproteins with chemical reporters,” J Am Chem Soc. 132:10628-1062.

The uncatalyzed 1,3-dipolar cycloaddition reaction of standard azides and alkynes is highly specific, but quite slow without catalysis. The Cu(I) catalysis of the reaction between azides and terminal alkynes was first described independently in 2002 in Tornøe C W, Christensen C, Meldal M. (2002) “Peptidotriazoles on Solid Phase: [1,2,3]-Triazoles by Regiospecific Copper (I)-Catalyzed 1,3-Dipolar Cycloadditions of Terminal Alkynes to Azides,” J. Org. Chem. 67:3057-3062; and Rostovtsev V V, Green L G, Fokin V V, Sharpless K B (2002) “A Stepwise Huisgen Cycloaddition Process: Copper (I)-Catalyzed Regioselective Ligation of Azides and Terminal Alkynes,” Angew Chem, Int Ed. 41:2596-2599. Another general solution to the azide-alkyne reaction rate problem is to make the alkyne highly strained in a ring structure. Such reactions are now referred to as “copper-free click chemistry.” See, for example, Codelli J A, Baskin J M, Agard N J, Bertozzi C R (2008) “Second-Generation Difluorinated Cyclooctynes for Copper-Free Click Chemistry,” J. Am. Chem. Soc. 130 (34): 11486-11493.

As flexible and useful as it is, click chemistry still requires that one of the molecules contains an azide and the other an alkyne. As noted above, affixing one or the other of these reactive groups to biomolecules has proven troublesome.

A 1963 paper to F. Minisci and R. Galli, “Reactivity of Hydroxy and Alkoxy Radiscals in Presence of Olefins and Oxidation-Reduction Systems, Introduction of Azido, Chloro, and Acyloxy Groups in Allylic Position and Azido-Chlorination of Olefins,” Tetrahedron Letters 6:357-360, describes an azide/halogen radical addition to ring structures, specifically across a double bond (from page 358 of the paper):

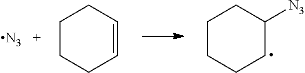 [SMILES: C1=CCCCC1 ; [N-]=[N+]=NC1CCCCC1 ; "·N3"]

Fenton's reagent (a mixture of H₂O₂ and iron (II) sulfate) was used. But neither amino acids, nor proteins, nor nucleic acids are mentioned.

Singh A and Koroll G W (1982) “Pulse Radiolysis of Aqueous Solutions of Sodium Azide: Reactions of Azide Radical With Tryptophan And Tyrosine,” Radiat. Phys. Chem. 19 (2): 137-146, speculates that an azide might add to aromatic amino acids. However, in this work, H₂O₂ was generated as a byproduct of radiolysis. The authors suggest that the H₂O₂ contributes to azide radical quenching and destruction. A directly contrary paper appeared in 1984: Butler J, Land E J, Swallow A J (1984) “The Azide Radical and Its Reaction with Tryptophan and Tyrosine,” Radiat Phys Chem 23 (1-2): 265-270. This paper cites to Singh and Koroll (1982), and strongly concludes “We, therefore, disagree that there is evidence that N* adds to TrpH.”

This same conclusion was drawn in Gatin, Billault, Duchambon, Van der Rest, Sicard-Roselli (2021) “Oxidative radicals (HO· or N3·) induce several di-tyrosine bridge isomers at the protein scale,” Free Radical Biology and Medicine 162:461-470. Here, the authors explored dimerization of peptides and proteins via azide radical-generated tyrosine radicals. The authors note that in treating tyrosine with hydroxyl radicals, the evidence shows addition of —OH onto the aromatic ring. But when the corresponding reaction is run with azide radicals, they saw no evidence of the addition of azide groups onto rings, “[N]o N3· addition on the ring was reported.” In short, this paper mentions the interaction of azide radicals with amino acids solely in the context of generating amino acid radicals to study dimerization of the amino acid radicals.

Thus, there remains a long-felt and unmet need for a fast, easy, and direct method to azidylate biomolecules, including proteins, polypeptides, and nucleic acids. Such a method would enable access to the panoply of reactions that can be accomplished using click chemistry.

### SUMMARY OF THE INVENTION

Disclosed herein are the following:
1. A method of attaching an azide moiety to a biomolecule, the method comprising contacting a biomolecule in a solution with an azide and an oxidizing agent, for a time and at a temperature wherein at least one azide moiety is bonded to the biomolecule to yield an azidylated biomolecule.
2. The method of claim 1, wherein the biomolecule is a protein.
3. The method of claim 2, wherein the protein is an intracellular protein.
4. The method of claim 2, wherein the protein is a membrane-bound protein.
5. The method of claim 2, wherein the protein is a circulating protein.
6. The method of claim 2, wherein the protein is an antibody.
7. The method of claim 1, wherein the biomolecule is a nucleic acid polymer.
8. The method of claim 7, wherein the nucleic acid polymer is a DNA polymer or a RNA polymer.
9. The method of any one of claims 1-8, wherein the solution is an aqueous solution.
10. The method of any one of claims 1-8, wherein the solution is a non-aqueous solution.
11. The method of any one of claims 1-10, wherein the oxidizing agent comprises H₂O₂.
12. The method of any one of claims 1-10, wherein the oxidizing agent consists of H₂O₂.
13. The method of any one of claims 1-10, wherein the oxidizing agent comprises phenyliodosohydroxy tosylate.
14. The method of any preceding claims, comprising contacting the biomolecule with the azide for 1 second to 1 hour, at a temperature of from 4° C. to 100° C.
15. The method of any preceding claim, further comprising reacting the azidylated biomolecule with a reagent comprising an alkyne.
16. The method of claim 15, wherein the alkyne is a terminal alkyne.
17 The method of claim 15, wherein the alkyne is an internal alkyne.
18. The method of claim 15, wherein the reagent comprising an alkyne is a cyclic alkyne.
19 The method of claim 15, wherein the reaction with an alkyne is a CuAAC reaction.
20. The method of claim 15, wherein the reaction with an alkyne is a SPAAC reaction.
21. A method of attaching a reagent comprising an alkyne to a biomolecule, the method comprising reacting a biomolecule in a solution with an azide and a reagent comprising an alkyne, for a time and at a temperature wherein at least a some of the reagent comprising an alkyne is covalently bonded to the biomolecule via a triazole linkage.
22. The method of claim 21, wherein the alkyne is a terminal alkyne.
23 The method of claim 21, wherein the alkyne is an internal alkyne.
24 The method of claim 21, wherein the reagent comprising an alkyne is a cyclic alkyne.
25. The method of claim 21, wherein the biomolecule is a protein.
26 The method of claim 25, wherein the protein is an intracellular protein.
27 The method of claim 25, wherein the protein is a membrane-bound protein.
28. The method of claim 25, wherein the protein is a circulating protein.
29. The method of claim 25, wherein the protein is an antibody.
30 The method of claim 25, wherein the protein comprises aromatic residues.
31. The method of claim 25, wherein the protein is bovine serum albumin.
32. The method of claim 21, wherein the biomolecule is a nucleic acid polymer.
33. The method of claim 32, wherein the nucleic acid polymer is a DNA polymer or a RNA polymer.
34 The method of any one of claims 21-33, wherein the solution is an aqueous solution.
35. The method of any one of claims 21-33, wherein the solution is a non-aqueous solution.
36. The method of any one of claims 21-35, wherein the reaction is a CuAAC reaction.
37. The method of any one of claims 21-35, wherein the reaction is a SPAAC reaction.
38. The method of any one of claims 21-37, comprising conducting the reaction for about 1 second to about 1 hour, at a temperature of from about 4° C. to about 100° C.
39 The method of any one of claims 21-38, further comprising vortexing the vessel in which the reaction occurs during the reaction.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A. The azide anion and azido radical triatomic species exhibit molecular resonance, whereas the diatomic hydroxyl radical does not.

FIG. 1B. The photocleavable, biotinylated alkyne used to modify, enrich, release, and detect points of azidylation. Photocleavage with 365 nm light occurs across the bond indicated with the red line, and following the click reaction, results in the triazole product shown in FIG. 1C.

FIG. 1C. The general workflow and mass shift detectable on peptides following the listed steps.

FIG. 1D. The full illustrated experimental workflow, step-by-step.

FIG. 2A. Gel-based detection of azidylation of bovine serum albumin (“BSA”) as demonstrated by fluorescent click chemistry and blotting after SDS-PAGE. The top panel is a streptavidin blot using a copper and alkyne-based conjugation system visualized by fluorescent anti-streptavidin antibody. Below is shown a similar gel stained for protein with Coomassie Blue, demonstrating equal loading of BSA per lane. Ladders and lanes are from separate regions of the same gels and have been cropped but not vertically shifted (grey and black lines, respectively). Background fluorescence is observed to a small degree in lanes without peroxide and azide, but this background is not observed via mass spectrometic analysis of conjugated peptides following enrichment as shown in bar graph.

FIG. 2B. BSA azidylation is azide dose-responsive, and at lower concentrations peroxide causes increased azidylation over that with azide alone. As in FIG. 2A, the top panel is a streptavidin blot, and below is Coomassie staining of the same samples demonstrating equal loading. Below, similar levels of azidylation are observed via mass spectrometry with 100 mM azide+/−peroxide, lower levels with 10 mM azide and added peroxide, and higher levels with 1 mM azide and added peroxide.

FIG. 3. Adding hydrogen peroxide, sodium azide, or the two together does not increased observed oxidation (+16) on BSA.

FIG. 4. Left panel: GFP fluorescence is not lost under reaction conditions used for BSA and lysozyme. Right panel: If reaction continues a minute longer, more loss of fluorescence is seen in azide containing samples, right. Error bars represent standard deviation, n=3 per sample.

FIG. 5A. Unlike BSA, both azide and peroxide are necessary for direct protein azidylation of lysozyme. The top panel shows a streptavidin blot; the middle panel is Coomassie staining of the same samples demonstrating equal loading. An increase in blotting is observed with added peroxide alone. Lysozyme does not exhibit the same properties as BSA; peroxide and azide together are necessary for azidylation.

FIG. 5B. Small amounts of azide titrated into the copper-catalyzed azide-alkyne cycloaddition reaction cause azidylation of BSA. Ladders and lanes are from separate regions of the same gels and have been cropped but not vertically shifted (grey and black lines, respectively). Increases in streptavidin reactivity correlate with increased azidylation as observed with mass spectrometry (graph). Whereas 1 mM or 10 mM azide yield approximately the same amount of azidylation, none is observed with 10 μM. Again, the top panel shows the streptavidin blot; the middle panel the Coomassie-stained gel.

FIG. 5C. Palmitate titrated into the azidylation reaction inhibits BSA azidylation. Shown above is fluorescence clicked onto BSA post-azidylation and shown below is a coomassie stain of the same gel. Palmitate, even at the lowest concentration tested, inhibits direct azidylation of BSA.

FIG. 5D. In human serum albumin co-crystallized with palmitate, sites analogous to those heavily azidylated in BSA sit directly in a palmitate binding pocket. Shown in blue, HSA. Shown in red, co-crystallized palmitate. Shown in orange sticks, residues homologous to azidylation sites observed in BSA. Three azide sites homologous to BSA sit within 11 Å each of bound lipids.

FIG. 5E. The working model for DACC. Azide: protein interaction is coordinated both by aromatic amino acids and hydrophobic interactions, and supplying an oxidant, in this case H₂O₂ or reagents for CuAAC, causes azidylation. Proposed end products of azidylation on confirmed residues are shown. For tryptophan and lysine, single modification on different carbons throughout the ring or hydrocarbon chain may be occurring, only one is shown as an example.

FIG. 6. Replicate 1 of intact BSA treated with 100 mM azide and 1% H₂O₂, with putative sites and when possible, site probabilities, displayed above the sequence.

FIG. 7. Replicate 2 of intact BSA treated with 100 mM szide and 1% H₂O₂, with putative sites and when possible, site probabilities, displayed above the sequence.

FIG. 8. Replicate 3 of intact BSA treated with 100 mM szide and 1% H₂O₂, with putative sites and when possible, site probabilities, displayed above the sequence.

FIG. 9. Replicate 1 of digested BSA peptides treated with 100 mM azide and 1% H₂O₂. No azidylation was identified.

FIG. 10. Replicate 2 of digested BSA peptides treated with 100 mM azide and 1% H₂O₂. No azidylation was identified.

FIG. 11. Replicate 3 of digested BSA peptides treated with 100 mM azide and 1% H₂O₂. No azidylation was identified.

FIG. 12. Lysozyme structure (8LYZ) is shown with hydrophobic patch residues shown in orange and sticks. The two most azidylated tryptophans (W108, left, and W123, right) are shown as sticks and in blue.

FIG. 13A depicts the general azidylation reaction disclosed herein. Bovine serum albumin (“BSA”) was treated with hydrogen peroxide and azide to azidylate the BSA. Following this, click chemistry was used to attach a photocleavable biotin tag to the azidylated BSA via the labeled terminal alkyne. A concurrent control reaction was performed without added alkyne. Attaching the biotin tag to BSA allowed visualization of the azidylated, biotinylated BSA using Western blotting with streptavidin to detect the attached biotin. The results are shown in the gel of FIG. 13B.

FIG. 13B is a photograph of parallel gels demonstrating the biotinylation of the BSA by click chemistry as described in FIG. 13A. On the left in FIG. 13B is a Coomassie-stained western blot demonstrating large amounts of BSA in both mock (−) and alkyne-treated (+) samples. A simultaneously run western blot is shown on the right demonstrating that large amounts of biotin are only detected BSA that has been azidylated and “clicked” to the alkyne in A (+) versus the mock reaction (−).

FIG. 14A depicts a non-limiting, exemplary workflow following the “click” reaction of the alkyne reagent to the azidylated protein (in this example, BSA). Proteins were digested to peptides. The resulting peptides derived from BSA that include the photocleavable biotin tag were bound to streptavididn resin, followed by elution/cleavage with 365 nm light. This produced the mass adduct triazole shown in FIG. 14A. The location and amount of the adduct was assayed with targeted bioinformatic searches following mass spectrometry data acquisition. Mass spectrometry (“MS”) results are shown in FIG. 14B (no alkyne) and FIG. 14C (with alkyne).

FIG. 14B shows the mass spectrometry results when no alkyne is added to the reaction.

FIG. 14C shows the mass spectrometry results when the biotinylated alkyne is added to the reaction. As shown in FIGS. 14B and 14C, there was significantly more mass adduct tag when the azidylated BSA was subjected to the click reaction, digested, and enriched as described in FIG. 14A. There was virtually no mass adduct tag when the mock reaction was run without the biotinylated alkyne. FIGS. 14B and 14C both show the sequence of BSA. Highlighted in green are peptides identified by mass spectrometry. Above the sequences, O designates standard oxidation of the corresponding residue. C designates standard carbamidomethylation of the corresponding residue. P (further highlighted with a red arrow) designates the mass adduct shown in FIG. 14A (+96.04 amu). In all of FIGS. 14A, 14B, and 14C, the BSA was azidilyated using 500 mM azide and 1.0% H₂O₂ in water, at ambient temperature. (See Examples, below.)

FIG. 15A depicts a non-limiting example of a paired control reaction. Proteins were digested to peptides. Peptides from BSA that included the photocleavable biotin tag (“BSA-PCtag-Biotin” in the figure) added via the “click” reaction were bound to streptavididn resin, followed by elution/cleavage with 365 nm light. The unbound fractions from the steptavidin enrichment were also analyzed via MS as a paired control.

FIG. 15B depicts the MS results for the unbound fraction. FIG. 15C depicts the MS results for the enriched fraction. As shown in FIG. 15B, no mass adduct tag is found in the unbound fraction of the enrichment. In contrast, a significant amount of the adduct tag in the enriched fraction; FIG. 15C. In the same fashion as for FIGS. 14B and 14C, FIGS. 15B and 15C show the sequence of BSA. Highlighted in green are the peptides identified by MS. Above the sequences, O designates standard oxidation of the corresponding residue, C designates standard carbamidomethylation of the corresponding residue, and P (further highlighted with a red arrow) designates the mass adduct shown in FIG. 14A (+96.04 amu). Here, 100 mM azide and 1.0% H₂O₂ were used for azidylation.

FIGS. 16A and 16B: Clickability above background in Arabidopsis cytosolic extract is H₂O₂— and azide-dependent. Samples were clicked with CuAAC chemistry following azidylation to AZDye 680 Alkyne. See the Examples. FIG. 16A shows fluorescent imaging of the gel. FIGS. 16B and 16B, Coomassie stain of same gel.

FIGS. 17A and 17B: BSA clickability is both azide- and phenyliodosohydroxy tosylate (PT)-dependent. Dose-dependent clickability is shown for both reagents. DBCO AzDye800 was clicked onto protein following azidylation via SPAAC chemistry, and fluorescently imaged gels are shown on top. Below, the same gels used for fluorescent imaging were dyed with Coomassie blue stain and protein load was assayed. FIG. 17A: Azide is kept constant at 100 mM and dosage of PT is varied. Under 20 μM PT does not produce clickability above background under the conditions used. PT concentrations were as shown. FIG. 17B: PT is kept constant at 200 μM and dosage of azide is varied. The lowest concentration used, 10 μM, produces clickability above background.

FIGS. 18A and 18B show percentage of azidylated lysozyme with azide dosages of 1 mM, 10 mM, 100 mM, and 500 mM, and with/without H₂O₂. FIG. 18B shows the peptide spectral match (“PSM”) count of the total and modified lysozyme, and the calculated percentage of the modified lysozyme that corresponds to the percentage shown in FIG. 18A.

FIGS. 19A-19D show sequence coverage maps following the “click” reaction to attach biotinylated alkyne to products obtained from the azidylation reactions shown in FIGS. 19A and 19B. Highlighted in green are peptides identified by mass spectrometry. Above the sequences, “O” designates standard oxidation of the corresponding residue. “C” designates standard carbamidomethylation of the corresponding residue. “P” designates the mass adduct of the PC biotin tag.

FIG. 20 depicts a non-limiting, exemplary “one-pot” click reaction. Protein A is mixed with standard click reagents, sodium azide, and an alkyne-chemical group B. The reaction is conducted for 20 min, at room temperature, in the dark, and without mixing, to covalently bond protein A to chemical group B via a clicked triazole linkage.

FIG. 21 shows percentage of modified BSA following the “one-pot” click reaction with 10 μM, 100 μM, 1 mM and 10 mM azide, without and with vortexing during the reaction.

FIGS. 22A-22D show sequence coverage maps following the “one-pot” click reactions of the biotinylated alkyne, azide, and BSA conducted in conditions shown in FIG. 21. Highlighted in green are peptides identified by mass spectrometry. Above the sequences, “O” designates standard oxidation of the corresponding residue. “C” designates standard carbamidomethylation of the corresponding residue. “P” designates the mass adduct of the PC biotin tag.

FIGS. 23A-23D. Methodology for oxidative azidylation and detection via mass spectrometry. FIG. 23A: The diatomic hydroxyl radical, created with energy input, has no delocalized resonance, the triatomic azide free radical delocalizes electron density via resonance. FIGS. 23B and 23C: Alkynes used in conjunction with copper-catalyzed azide-alkyne cycloaddition (CuAAC) for detection, enrichment, and mapping of azide covalently bound to protein. FIG. 23D: Schematic of methodology for detection of covalent azidylation, as described in main text.

FIGS. 24A-24E. Lysozyme and BSA are azidylated in an azide dose-dependent fashion. FIG. 24A: Both azide and peroxide are necessary for strong, direct protein azidylation of lysozyme. Shown above is a streptavidin blot, and below is Coomassie staining of the same samples demonstrating equal loading. An increase in blotting is observed with added peroxide alone. FIG. 24B: Increased azidylation is observed when hydrogen peroxide is added to lysozyme and azide. FIG. 24C: Lysozyme (PDB: 8LYZ) shown in dark gray, and every azidylated residue shown in orange and labelled with residue number. FIG. 24D: Gel-based detection of azidylation of BSA as demonstrated by fluorescent click chemistry and blotting after SDS-PAGE. Shown above is a streptavidin blot using a copper and alkynebased conjugation system visualized by fluorescent anti-streptavidin antibody. Below is shown a similar gel stained for protein with Coomassie Blue, demonstrating equal loading of BSA per lane. Ladders and lanes are from separate regions of the same gels and have been cropped but not vertically shifted (grey and black line, respectively). Background fluorescence is observed to a small degree in lanes without peroxide and azide, but this background is not observed via mass spectrometric analysis of conjugated peptides following enrichment as shown in bar graph. FIG. 24E: BSA azidylation is azide dose-responsive, and at lower concentrations peroxide causes increased azidylation over that with azide alone. As in FIG. 24A, shown above is a streptavidin blot, and below is Coomassie staining of the same samples demonstrating equal loading. Below, similar levels of azidylation are observed via mass spectrometry with 100 mM azide+/−peroxide, lower levels with 10 mM azide and added peroxide, and higher levels with 1 mM azide and added peroxide.

FIG. 25. GFP fluorescence is not lost under reaction conditions used for BSA and lysozyme, left. If reaction continues a minute longer, more loss of fluorescence is seen in azide containing samples, right. Error bars represent standard deviation, n=3 per sample.

FIG. 26. Adding hydrogen peroxide, sodium azide, or the two together does not increased observed oxidation (+16) on BSA.

FIGS. 27A-27E. Azidylation requires 3D structure and can be outcompeted with hydrophobic ligands. FIG. 27A: Digesting BSA to tryptic peptides prior to hydroxyl radical footprinting increases modification, as expected given its correlation to solvent accessibility; digesting BSA prior to azidylation abolishes modification entirely. FIG. 27B: Similar to azide, digesting BSA to peptides ameliorates ANS binding and fluorescence. Left to right, empty digestion buffer (50 mM ammonium bicarbonate), 1.5 μM BSA prior to adding protease for digestion, 1.5 μM protease after 4 hours at 42° C. with no protease added, 1.5 μM protease after 4 hours digestion at 42° C., and 1.5 μM BSA peptides after overnight digestion, desalting, and resolubilization into digestion buffer. FIG. 27C: Digesting BSA ameliorates ANS binding and fluorescence in a time-dependent fashion. Data points are averages and error bars are standard deviation from n=3 fluorescent measurements of single samples. FIG. 27D: Lysozyme azidylation can be ameliorated by first adding and equilibrating the noncovalent hydrophobic probe ANS. Shown above is a streptavidin blot, and below is Coomassie staining of the same samples demonstrating equal loading. FIG. 27E: BSA azidylation can be ameliorated by first adding and equilibrating palmitate to BSA, which binds within buried pockets. Here, fluorescence, rather than biotin, was clicked onto BSA using the CuAAC reaction, and the gel was fluorescently imaged, then Coomassie stained and imaged a second time.

FIG. 28. Three replicates of intact BSA treated with 100 mM Azide and 1% H₂O₂, with putative sites and when possible, site probabilities, displayed above the sequence.

FIG. 29. Three replicates of digested BSA peptides treated with 100 mM Azide and 1% H₂O₂. No azidylation was identified.

FIG. 30. Fully digested BSA peptides do not bind to ANS and do not cause ANS fluorescence, and may inhibit at higher concentrations, though this could be due to trace salts not removed with the desalting procedure. All experiments were performed in 50 mM ammonium bicarbonate. Data points are the average for n=3 measurements and error bars are +/−standard deviation.

FIGS. 31A-31G. Azidylation occurs in solvent inaccessible regions, and in Arabidopsis tissue lysate, falls within active sites and known azide binding sites. FIG. 31A: Azidylated residues (orange) in lysozyme line a contiguous, buried cleft, and are aligned with one another (PDB: 8LYZ). FIG. 31B: Azidylated residues in BSA (orange) are near two bound palmitate molecules (blue in HSA co-crystallized with palmitate (PDB: 1E7H). FIG. 31C: On rubisco, the two strongest sites of azidylation (shown in orange) are buried within the active site, and in direct contact with transition state analogue 2-carboxyarabinitol-1,5-bishosphate (shown in blue) in the crystal structure (PDB: 5IU0). FIG. 31D: On catalase, the strongest site of azidylation is Y407 (shown in orange), which is conserved in Bos taurus catalase (shown here with azide co-crystallized), coordinates the heme group, and is <5 Å from azide bound in the crystal structure (PDB: 1TH2). The heme-bound iron has been removed for better viewing. FIG. 31E: The site of azidylation identified on Cu/Zn superoxide dismutase, V123, is conserved in Saccharomyces cerevisiae (shown here with azide co-crystallized) and is ˜6 Å from the azide bound in the crystal structure (PDB: 1YAZ). FIG. 31F: Azidylation on rubisco's large subunit only occurs on solvent inaccessible regions when the reaction is performed in a clarified Arabidopsis tissue lysate. Relative solvent accessible surface area (SASA) is shown from N- to C-terminus and orange dots are localized azidylated residues or short stretches. FIG. 31G: The cumulative data suggest that azide binds to three-dimensional hydrophobic regions on protein (shown as red in this hypothetical diagram). This binding occurs first noncovalently, as shown in middle images. Oxidation then radicalizes azide into the azido radical, which attacks and covalently modify amino acid side chains at binding sites, as shown on the right diagram.

FIG. 32. Azidylation on lysozyme in highly enriched in solvent inaccessible regions. Relative solvent accessible surface area (SASA) is shown from N- to C-terminus and orange dots are localized azidylated residues or short stretches

FIG. 33. The strongest azidylation identified on Arabidopsis rubisco corresponds to conserved histidines in close proximity to bound CO₂ (left, PDB: 4FOK) and O₂ (right, PDB: 4FOH) in Galdiera rubisco.

FIG. 34. Azidylation on catalase only occurs on solvent inaccessible regions when the reaction is performed in a clarified Arabidopsis tissue lysate. Relative solvent accessible surface area (SASA) is shown from N- to Cterminus and orange dots are localized azidylated residues or short stretches.

FIG. 35. Azidylation on Cu/Zn superoxide dismutase only occurs on one solvent inaccessible region when the reaction is performed in a clarified Arabidopsis tissue lysate. Relative solvent accessible surface area (SASA) is shown from N- to C-terminus and orange dots are localized azidylated residues or short stretches.

### DETAILED DESCRIPTION

### Abbreviations and Definitions

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 1 to 9, from 3.6 to 4.6, from 3.5 to 9.9, and so forth.

All references to singular characteristics or limitations shall include the corresponding plural characteristic or limitation, and vice-versa, unless otherwise specified or clearly implied to the contrary by the context in which the reference is made. That is, unless specifically stated to the contrary, “a” and “an” mean “one or more.” The phrase “one or more” is readily understood by one of skill in the art, particularly when read in context of its usage. For example, “one or more” substituents on a phenyl ring designates one to five substituents.

All combinations of method or process steps as used herein can be performed in any order, unless otherwise specified or clearly implied to the contrary by the context in which the referenced combination is made.

The methods disclosed herein can comprise, consist of, or consist essentially of the essential elements and limitations of the method as described herein, as well as any additional or optional ingredients, components, or limitations described herein or otherwise useful in synthetic organic chemistry.

The term “biomolecule” is defined broadly herein to encompass both small and macromolecular molecules found in nature, explicitly including, but not limited to proteins and polypeptides (terms which are used synonymously herein) and polynucleic acids of all types (e.g., DNA, RNA, and combinations thereof). Also included within the term are non-natural modified versions thereof, such as proteins with non-natural residues, tagged and labeled versions of natural biomolecules, etc. Non-limiting examples of “biomolecules” include antibodies, serum proteins, membrane-bound proteins, intracellular proteins and nucleic acids, genomic DNA, mRNA, tRNA, shRNA, etc.

The term “contacting” refers to the act of touching, making contact, or of bringing to immediate or close proximity, including at the molecular level, for example, to bring about a chemical reaction, or a physical change, e.g., in a solution or in a reaction mixture.

An “effective amount” refers to an amount of a chemical or reagent effective to facilitate a chemical reaction between two or more reaction components, and/or to bring about a recited effect. Thus, an “effective amount” generally means an amount that provides the desired effect.

The terms “label” and “labeled” are defined broadly herein to encompass any and all molecular markers, labels, or probes of any structure or configuration, now known or developed in the future, that can be detected by any means (now known or developed in the future). The term “label” as used herein is synonymous terms such as “marker” and “probe” and others that are conventionally encountered in the relevant literature. The term “label” includes, without limitation, radioactive labels, fluorescent labels, chromophoric labels, affinity-based labels (such as antibody-type markers, biotin, etc.), and the like. Conventional radioactive isotopes used for detection include, without limitation, ³²P, ¹³C, ²H, and many others. A huge number of fluorescent and chromophoric probes are known in the art and commercially available from numerous worldwide suppliers, including Life Technologies (Carlsbad, California, USA), Enzo Life Sciences (Farmingdale, New York, USA), and Millipore Sigma (also known as Sigma-Aldrich (St. Louis, Missouri, USA).

The term “solvent” refers to any liquid that can dissolve a compound to form a solution, without limitation. Solvents include water and various organic solvents, such as hydrocarbon solvents, for example, alkanes and aryl solvents, as well as halo-alkane solvents. Examples include hexanes, benzene, toluene, xylenes, chloroform, methylene chloride, dichloroethane, and alcoholic solvents such as methanol, ethanol, propanol, isopropanol, and linear or branched (see or tert) butanol, and the like. Aprotic solvents that can be used in the method include, but are not limited to perfluorohexane, α,α,α-trifluorotoluene, pentane, hexane, cyclohexane, methylcyclohexane, decalin, dioxane, carbon tetrachloride, freon-11, benzene, toluene, triethyl amine, carbon disulfide, diisopropyl ether, diethyl ether, t-butyl methyl ether (MTBE), chloroform, ethyl acetate, 1,2-dimethoxyethane (glyme), 2-methoxyethyl ether (diglyme), tetrahydrofuran (THF), methylene chloride, pyridine, 2-butanone (MEK), acetone, hexamethylphosphoramide, N-methylpyrrolidinone (NMP), nitromethane, dimethylformamide (DMF), acetonitrile, sulfolane, dimethyl sulfoxide (DMSO), propylene carbonate, and the like.

### The Method:

Disclosed herein is a simple method for directly and rapidly attaching azide groups (N3, —N═N═N) to biomolecules in solution in seconds, using widely available solvents. The method comprises contacting a biomolecule in solution (preferably, but not limited to aqueous solutions) with an azide and an oxidizing agent, for a time and under conditions such that azide moieties are covalently attached to the biomolecule. The biomolecules so azidylated can then optionally be further modified in any number of ways using click chemistry via the CuAAC or SPAAC reactions. Importantly, it has been verified by experimentation that the method disclosed herein attaches azide to protein. The azidylated protein can be further modified to include markers added via click chemistry. Detection can be accomplished by any suitable means, such as Western blotting. The reaction has further been confirmed by detection (via mass spectrometry) of the predicted mass adduct resulting from the click chemistry reactions.

The reaction is very straightforward. A biomolecule in solution is contacted with an azide and an oxidizing agent. This is done for a time (about 1 second to about 1 hour) and at a temperature (ambient is preferred, but from about 4° C. to about 100° C.) wherein at least one azide moiety is covalently bonded to the biomolecule. The reaction yields an azidylated biomolecule. The azidiylated biomolecule may then optionally be reacted with a reagent comprising an alkyne.

Also disclosed herein is a “one-pot” click method which allows attaching an alkyne-containing reagent to a biomolecule via a clicked triazole linkage in a single step. The method comprises reacting a biomolecule in a solution with an azide and a reagent comprising an alkyne, for a time and at a temperature wherein at least one reagent comprising an alkyne is covalently bonded to the biomolecules via a triazole linkage. The reaction can be conducted using any standard click reagents, such as CuAAC and SPAAC. It has been verified by experimentation that the method disclosed herein covalently attached alkyne-containing molecule to protein. The reaction is conducted for a time (about 1 second to about 1 hour) and at a temperature (ambient is preferred, but from about 4° C. to about 100° C.). It is preferred that the reaction is conducted with very gentle mixing (or without mixing) to minimize protein cleavage.

### Click-Chemistry:

As used herein, the term “click chemistry” is used to refer generically and broadly to a family of azide-alkyne cyclo-addition reactions, including (by way of example and not limitation) copper (I)-catalyzed azide-alkyne cycloaddition (hereinafter “CuAAC”) and strain-promoted azide-alkyne cycloaddition (hereinafter “SPAAC”), which does not require a copper (I) containing catalyst.

Click chemistry is a set of rapid and specific reactions for assembling fragments into more complex structures. The archetypical click reaction is the cycloaddition of azides and alkynes to form 1,2,3-triazoles, originally discovered by Rolf Huisgen. Barry Sharpless and Morten Meldal found that copper catalysts rendered the cycloaddition more selective and facile, making it useful for preparing small molecules, pharmaceuticals, antibodies, and polymers. Carolyn Bertozzi and her group have also been instrumental in developing click chemistry that does not require a copper catalyst.

Bioorthogonal reactions are a subset of click reactions useful for chemistry in living things; they must assemble molecules rapidly and selectively at low concentrations in water and at near-ambient temperatures. The term “bioorthogonal” was coined by Dr. Bertozzi in the early 2000 s; she and her research group developed two of the first biorthogonal reactions, the Staudinger ligation and strain-promoted azide-alkyne cycloadditions, i.e. SPAAC. This was a monumental advancement in the study of biological systems because copper is often toxic to cells.

The CuAAC reaction has been widely reported in the scientific literature. See, for example, Presolski, Hong, and Finn (2011) “Copper-Catalyzed Azide-Alkyne Click Chemistry for Bioconjugation,” Curr Protoc Chem Biol. 3 (4): 153-162, which is incorporated herein by reference.

The CuAAC reaction proceeds generally by the following reaction scheme:

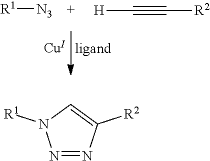

The basic CuAAC reaction requires only copper ions in the +1 oxidation state. These may be supplied by a discrete Cu(I) complex, by metallic copper, or copper-impregnated materials. See, for example, Rostovtsev V V, Green L G, Fokin V V, Sharpless K B. (2002) “A Stepwise Huisgen Cycloaddition Process: Copper (I)-Catalyzed Regioselective Ligation of Azides and Terminal Alkynes,” Angew Chem, Int Ed. 41:2596-2599. See also Lipshutz B H, Frieman B A, Tomaso A E., J (2006) “Copper-in-Charcoal (Cu/C): Heterogeneous, Copper-Catalyzed Asymmetric Hydrosilylations,” Angew Chem, Int Ed. 45:1259-1264. The reaction is also widely practiced using a mixture of a Cu(II) salt and a reducing agent, sodium ascorbate being the most popular (see, e.g., Rostovtsev et al. 2002, supra). Optionally, accelerating ligands may also be added to the reaction. These accelerating ligands act as chelating agents to maintain a readily available concentration of Cu(I) in solution. (Copper ions are quite facile and can undergo redox and disproportionation reactions that rapidly decrease the concentration of Cu(I) in the reaction solution.)

The CuAAC reaction has a host of benefits in the context of conjugating biomolecules. It yields a non-toxic triazole from biological building blocks that have been modified with non-perturbing azides and unactivated alkynes. The CuAAC reaction is reliable and tolerates a wide range of reaction conditions. It is pH-independent and can be carried out in water at ambient, room temperature. It can be utilized in reactions taking place entirely in solution and can also be utilized for solid-phase immobilization reactions. In the biomolecular realm in particular, azido groups and acetylenic groups are quite rare in natural biomolecules. Hence, the reaction is highly bio-orthogonal and specific.

The Cu(I)-free [2+3] cycloaddition, strain-promoted click strategy (SPAAC) relies on the use of strained dibenzylcyclooctynes (“DBCO's”). See, for example, Agard N J, Prescher, J A, and Bertozzi, CR (2004) “A strain-promoted [3+2]azide-alkyne cycloaddition for covalent modification of biomolecules in living systems,” J. Am. Chem. Soc. 126 (46): 15046-7. The strained conformation of DBCO's decreases the activation energy for the cycloaddition click reaction, enabling it to be carried out without the need for a catalyst. The reactions take place at low temperatures (ambient) with an efficiency greater than that of the Cu(I)-catalyzed ligation.

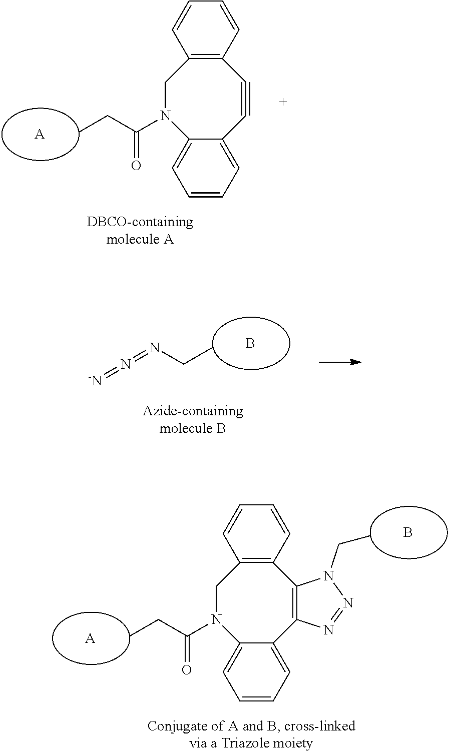

The Cu(I)-free ligation reaction scheme is shown schematically above. Diarylcyclooctyne-activated biomolecule A reacts with azide-activated biomolecule B without Cu(I) in aqueous conditions to form a stable triazole. Diarylcyclooctynes are thermally stable compounds with very narrow and specific reactivity toward azides. The ligation reaction is very fast and results in almost quantitative yield of stable triazoles.

A wide range of reagents for practicing click chemistry are available commercially from numerous international suppliers, including Millipore-Sigma, Inc. (Madison, Wisconsin, USA, a wholly owned subsidiary of Merck KGaA, Darmstadt, Germany), Interchim Inc. (San Pedro, California, USA), Interchim SA (Montluçon, France), and Cheshire Sciences Ltd., Chester, England. These reagents include a host of labeled alkynes and azides that permit a huge array of discovery-type and confirmatory-type reactions. These commercially available reagents include fluorescently labeled alkynes and azides, biotin-tagged alkynes and azide, and the like. For example, the following fluorescently labeled CuAAC reagents are available commercially from Interchim Inc.:
Alkyne-PEO₄-CR110: Fluor 488-Acetylene; C5/C6-carboxyfluorescein; Abs/Em=501/525 nm.
Alkyne-PEO₄-CR6G: Fluor 525-Acetylene; C5/C6-Carboxyrhodamine 6G; Abs/Em=522/544 nm.
Alkyne-PEO₄-TAMRA: Fluor 545-Acetylene; TMRA-PEO₄-Alkyne; MW: 644.73; Abs/Em=546/565 nm.
Alkyne-PEO₄-SRB: Fluor 568-Acetylene; Abs/Em=568/584 nm.
Alkyne-PEO₄—SR101 (with sulfo-propyl substituent): Fluor 585-Acetylene; Abs/Em=584/603 nm.
Alkyne-Trisulfo-Cyanine3: TrisulfoCy3-Acetylene; MW: 761.92; Abs/Em=550/570 nm.
Alkyne-Trisulfo-Cyanine5: TrisulfoCy5-Acetylene; MW: 787.96; Abs/Em=647/663 nm.
Alkyne-Trisulfo-Cyanine7 (with ethyl substituent): Trisulfo-Cy7-Acetylene; MW: 1010.22; Abs/Em=753/775 nm.
Alkyne-Disulfo-Cyanine3: Disulfo-Eth-CY7-Acetylene, CF₃CO₂ salt; MW: 781.86; Abs/Em=555/565 nm; soluble in DMSO.
Alkyne-Disulfo-Cyanine5: Disulfo-Eth-CY5-Acetylene, CF₃CO₂ salt; MW: 807.90; Abs/Em=649/66 nm; soluble in DMSO.
Alkyne-Tetrasulfo-Cyanine5.5: Tetrasulfo-Eth-CY5.5-Acetylene, CF₃CO₂ salt; MW: 1068.14; Abs/Em=578/701 nm; soluble in DMSO.
Alkyne-Disulfo-Cyanine7: Disulfo-Eth-CY7-Acetylene, CF₃CO₂ salt; MW: 833.93; Abs/Em=749/776 m; soluble in DMSO.
Alkyne-SulfoCyanine3: MonoCy3-Acetylene; MW: 573.75; Abs/Em=550/567 nm; extinction coefficient (“EC”)=96800; quantum yield (“QY”)=0.15.
Alkyne-Disulfo-Cyanine3: DisulfoCy3-Acetylene Na salt; MW: 675.79; Abs/Em=548/567 nm; EC=162000; QY=0.15.
Alkyne-Disulfo-Cyanine5: DisulfoCy5-Acetylene Na salt; MW: 701.83; Abs/Em=646/664 nm; EC=271000; QY=0.28.
Alkyne-Disulfo-Cyanine3: Disulfo-Cy3-Acetylene K salt; MW: 691.9; Abs/Em=548/563 nm; EC=162000; QY=0.1; CF260 nm=0.03; CF280 nm=0.06; soluble in water, DMF, and DMSO.
Alkyne-Disulfo-Cyanine3.5: Disulfo-Cy3.5-Acetylene.
Alkyne-Disulfo-Cyanine5: Disulfo-Cy5-Acetylene Potassium salt; MW: 717.94; Abs/Em=649/662 nm; EC=271000; QY=0.28; CF 260 nm=0.04; CF 280 nm=0.04; soluble in water, DMF, DMSO.
Alkyne-Tetrasulfo-Cyanine5.5: TetraSulfo-Cy5.5-Acetylene tri-K salt; MW: 1054.36; Abs/Em=673/691 nm; EC=195000; CF260 nm=0.09; CF280 nm=0.11; soluble in water, DMF, DMSO.
Alkyne-Disulfo-Cyanine7: Disulfo-Cy7-Acetylene K salt; MW: 745.3; Abs/Em=750/773 nm; EC=240600; CF₂₆₀ nm=0.04; CF₂₈₀ nm=0.04; Soluble in water, DMF, DMSO.
Alkyne-Tetrasulfo-Cyanine7.5: Tetrasulfo-CY7.5-Acetylene tri-K salt; MW: 1120.46; Abs/Em=778/797 nm; EC=222000; CF260 nm=0.09; CF280 nm=0.09; soluble in water, DMF, DMSO
Alkyne-CYanine3: Cy3-Acetylene; MW: 530.14; Abs/Em=555/570 nm; EC=150000; QY=0.31; CF260 nm=0.04; CF280 nm=0.09.
Alkyne-Cyanine5: Cy5-Acetylene; MW: 556.18; Abs/Em=646/662 nm; EC=250000; QY=0.2; CF260 nm=0.03; CF280 nm=0.04.
Alkyne-CYanine5.5: Cy5.5-Acetylene; MW: 656.30; Abs/Em=684/710 nm; EC=209000; QY=0.2; CF260 nm=0.02; CF280 nm=0.03.
Alkyne-CYanine7: Cy7-Acetylene; MW: 622.38; Abs/Em=750/773 nm; EC=199000; QY=0.23; CF260 nm=0.022; CF280 nm=0.029.
Alkyne-CYanine7.5: CY7.5-Acetylene; MW: 722.40; Abs/Em=788 nm/808 nm; EC=223000.

This is just a small sampling of the fluorescently labeled or otherwise functionally modified, commercially available CuAAC reagents on the market.

Biotin-labeled alkynes are also commercially available, such as acetylene-PEO₄-biotin, which can be purchased from Interchim and Millipore-Sigma.

Propargyl-CEP-oligonucleotides can also be used to attach azide-containing reporter groups such as biotin or fluorescent dyes by click chemistry. This allows the synthesis of highly modified DNA strands carrying multiple labels in a density that is not achieved by classic labeling techniques. (NB: “propargyl”=2-propynyl (HC≡C—CH₂—; CEP=cyanoethyl-N,N-diisopropyl phosphoramidite.) Commercially available reagents include
5-Propargyloxy-dU CEP
5-Octadiynyl-dU CEP
5-(Propargyloxy)-2′-deoxyuridine
5-(1,7-Octadiyn-1-yl)-2′-deoxyuridine
5′-O-(Dimethoxytrityl)-5-(propargyloxy)-2′-deoxyuridine
5-Octadiynyl-TMS-dU CEP
5-Octadiynyl-TMS-dC CEP
5-Octadiynyl-dC CEP
5-Octadiynyl-TIPS-dU CEP among many others.

In the same fashion as for CuAAC, there is a wide variety of commercially available reagents for copper-free SPAAC, including (by way of example and not limitation): dibenzylcyclooctyne-amine, dibenzylcyclooctyne-acid, dibenzylcyclooctyne-NHS ester, dibenzylcyclooctyne-S—S—NHS ester, dibenzylcyclooctyne-maleimide, sulfo-dibenzylcyclooctyne-NHS ester, dibenzylcyclooctyne-PEG₄-alcohol, dibenzylcyclooctyne-PEG₄-acid, dibenzylcyclooctyne-PEG₄-amine, dibenzylcyclooctyne-PEG₅-NHS ester, dibenzylcyclooctyne-PEG₄-maleimide, dibenzylcyclooctyne-biotin conjugate, dibenzylcyclooctyne-PEG₄-biotin conjugate, dibenzylcyclooctyne-PEG₁₂-biotin conjugate, dibenzylcyclooctyne-S—S-PEG₃-Biotin Conjugate, dibenzylcyclooctyne-S—S-PEG₁₁-biotin conjugate, and sulfo-dibenzylcyclooctyne-biotin conjugate.

A slew of fluorescently labeled or otherwise modified DBCO molecules are also commercially available from the above suppliers, including (among many others):
Dibenzylcyclooctyne-Fluor 488 Abs/Em=501/525 nm
Dibenzylcyclooctyne-Fluor 525 Abs/Em=522/544 nm
Dibenzylcyclooctyne-Fluor 545 Abs/Em=546/565 nm
Dibenzylcyclooctyne-Fluor 568 Abs/Em=568/584 nm
Dibenzylcyclooctyne-Fluor 585 Abs/Em=584/603 nm
Cy3-DBCO
Cy5-DBCO
Cy5.5-DBCO
Cy7-DBCO
Cy7.5-DBCO

By way of example, and not limitation, Millipore Sigma currently offers the following DBCO and related reagents:

| Millipore | |
|---|---|
| Sigma | |
| Cat. No. | Systematic Name |
| 762024 | Azide-PEG3-biotin conjugate |
| 745073 | N-[1R,8S,9s)-Bicyclo[6.1.0] |
| | non-4-yn-9-ylmethyloxycarbonyl]- |
| | 1,8-diamino-3.6-dioxaoctane |
| 742678 | (1R,8S,9s)-Bicyclo[6.1.0]non-4-yn- |
| | 9-ylmethanol |
| 744867 | (1R,8S,9s)-Bicyclo[6.1.0]non-4- |
| | yn-9-ylmethyl-N-succinimidyl |
| | carbonate |
| 777366 | DBCO-Cy3 |
| 777374 | DBCO-Cy5 |
| 761540 | Dibenzocyclooctyne-amine |
| 761524 | Dibenzocyclooctyne-N-hydroxy- |
| | succinimidyl ester |
| 761532 | Dibenzocyclooctyne-S-S-N- |
| | hydroxysuccinimidyl ester |
| 760668 | Dibenzocyclooctyne-maleimide |
| 759902 | Dibenzocyclooctyne-PEG4-acid |
| 761982 | Dibenzocyclooctyne-PEG4-alcohol |
| 760749 | Dibenzocyclooctyne-PEG4-biotin |
| | conjugate |
| 760773 | Dibenzocyclooctyne-PEG4-Fluor |
| | 545 |
| 760676 | Dibenzocyclooctyne-PEG4- |
| | maleimide |
| 762040 | Dibenzocyclooctyne-sulfo-N- |
| | hydroxysuccinimidyl ester |
| 760706 | Sulfo-dibenzocyclooctyne-biotin |
| | conjugate |

The flexibility and utility of copper-free click chemistry in the investigation of the interactions of biomolecules is manifest. For example, a novel class of difluorinated cyclooctyne (DIFO) reagents were employed in copper-free click chemistry for the site-selective labeling of biomolecules in vitro and in vivo. See Codelli J A, Baskin J M, Agard N J, Bertozzi C R. (2008) “Second-Generation Difluorinated Cyclooctynes for Copper-Free Click Chemistry,” J. Am. Chem. Soc. 130 (34): 11486-11493.

Catalyst-free click reactions are useful for preparing radiometal-based pharmaceuticals. Radiotracer [⁶⁴Cu]DOTA-ADIBON₃-Ala-PEG₂₈-A20FMDV2, used for positron emission tomography imaging of integrin αvβ6-expressing tumors, has been synthesized via copper-free click chemistry. Satpati D, Bauer N, Hausner S H, Sutcliffe J L (2014) “Synthesis of [⁶⁴Cu]DOTA-ADIBON₃-Ala-PEG₂₈-A20FMDV2 via copper-free click chemistry for PET imaging of integrin αvβ6,” J Radioanal Nucl Chem. 302 (2): 765-771.

Iodine radioisotope labeling of cyclooctyne-containing molecules by copper-free click reaction has been reported. Radioiodination using the tin precursor was carried out at room temperature to obtain ¹²⁵I-labeled azide. Dibenzocyclooctyne (DBCO)-containing cRGD peptide and gold nanoparticle were labeled by employing ¹²⁵I-labeled azide to afford triazoles in good radiochemical yields (67-95%). This method is useful for both in vitro and in vivo labeling of DBCO group-containing molecules with iodine radioisotopes. See Jeon J, Kang J A, Shim H E, Nam Y R, Yoon S, Kim H R, Lee D E, Park S H (2015) “Efficient method for iodine radioisotope labeling of cyclooctyne-containing molecules using strain-promoted copper-free click reaction,” Bioorganic & Medicinal Chemistry 23 (13): 3303-3308.

A protein, site-specific labeling techniques employing the SPAAC reaction between dibenzocyclooctyne-fluor 545 (DBCO-fluor 545) and an azide-bearing unnatural amino acid is described in Zhang G, Zheng S, Liu H, Chen P R. (2015) “Illuminating biological processes through site-specific protein labeling,” Chem. Soc. Rev. 44 (11): 3405-3417.

These types of click chemistry reactions, and many more, are made much easier using the presently disclosed method because the method allows for the fast and easy azidylation of a biomolecule of interest. Further, the standard click reagents of CuAAC or SPAAC can be used in the “one-pot” click method disclosed herein to attach an alkyne-containing reagent to a biomolecule via clicked triazole linkage in a single step.

### Utility:

The method can be used in a host of different ways to elucidate various biomolecular interactions. For example, the method can be used to measure solvent accessibility of full proteomes. The “clickability” of the attachment (i.e., does the alkyne-containing reagent react with the azidylated biomolecule targe, and if so, to what extent) means an enrichable tag can be clicked onto modified regions of biomolecules of interest. Currently, there is no easy way to measure solvent accessibility on a global scale with complex protein samples. This is mainly due to sampling issues within proteomes. As with other proteomic fields, enrichment is the answer. When applied as such, the method disclosed herein is useful to measure proteome-wide solvent accessibility.

The ability to measure proteome-wide solvent accessibility is valuable for academic research with the goal of understanding biological systems and mechanisms. These data are also extremely valuable to the pharmaceutical industry, for drug discovery in broader biological contexts. On top of being able to map drug binding sites in a complex proteome, the technique allows researchers to detect off-target protein sites to help modify drugs once a lead candidate is obtained.

In addition to drug discovery in the pharmaceutical industry, covalently attaching drugs or other conjugates to antibodies for targeted delivery in living systems is a growing field. Antibodies can easily be modified with azide using the method disclosed herein. Once so modified, the azidylated antibodies can be conjugated with an alkyne-modified pharmacologically active agent for targeted delivery of the active agent to a specific in vivo location. From a biotech industry standpoint, generating azidylated proteins for further modification view click chemistry has immense value due the specificity, robustness, and versatility of the CuAAC and SPAAC reactions.

The method is also vastly cheaper and easier than current alternative methods to create azidylated proteins. Currently, the only method to azidylated proteins that doesn't involve a significant amount of chemical derivatization with multiple steps is to have cell lines incorporate azido amino acids (of which only a handful exist) into protein as it is made in vivo. This is a long, cumbersome, expensive process that has no guarantee of yielding the desired azidylated protein product.

Additionally, the method is extremely useful to label proteins quickly and easily, with fluorescent labels, radioactive labels, any kind of label that can be modified to bear a reactive alkyne group. Currently such methods generally use fluorescently labeled lysine or cysteine reagents. Radioactive tyrosine iodination has also been used in the past. Current approaches also generate fusion proteins comprising green fluorescent protein (“GFP”) or some other proteinaceous fluorophore. Or amino acid-modifying reagents ar used, as mentioned above.

In contrast, the method disclosed herein can easily add fluorescence to proteins in vitro via the click reaction. For example, there are many labs doing reactive oxygen species (ROS) imaging under a microscope. The method disclosed herein enables imaging ROS using click chemistry (azide addition is dependent upon hydroxyl radical generation).

Another example of how direct, simple and fast azidylation of proteins is useful to the scientific community is in its use as a covalent labeling (CL) reagent. CLs are widely used to determine the three-dimensional shape of proteins in their native folded state. In essence, CLs react with amino acid residues in proteins based on their inherent reactivity with the 20 amino acid side chains. Importantly, CL's also react with amino acid residues based on the solvent-accessible surface area (SASA) of the protein. SASA is calculated from a 3D structure previously determined. (The 3D structure of the protein may be determined by any means now known or developed in the future, including, by way of example and not limitation, NMR, X-ray crystallography, and cryoEM.) SASA is typically measured by computationally rolling a ball the size of a water molecule across the protein's 3D structure and the depth to which the ball enters the protein's inner cavities and becomes close in proximity to a particular amino acid is the SASA value for that amino acid. This value closely corresponds to the reactivity observed with hydroxyl radical footprinting (HRF), one of the most widely used forms of CL. This is extremely important, for example, in the protein therapeutics industry, where the contact points between an antibody and an antigen are being mapped to single amino acid resolution. Once these 3D epitopes are mapped, comprehensive deep landscape mutagenesis can be used to improve the efficacy of binding and create, for example, a better antibody that has higher neutralizing capability for disease such as COVID (e.g. Regeneron monoclonal antibody attacking the spike protein on the outside of SARS CoV-2).

Because an azido radical is almost as small as a hydroxyl radical, our method for azidylating proteins may prove to be more widely used for analyzing protein 3D structure than HRF currently is.

### Use of the Method for Identifying Hydrophobic Microenvironmetns in Water-Soluble Proteins:

Disclosed herein is a method of identifying hydrophobic microenvironment in a soluble protein. The method comprises contacting the protein in a solution with an azide and an oxidizing agent, for a time and at a temperature wherein at least one azide moiety is covalently bonded to the protein, and localizing the binding of the azide using mass spectrometry.

Hydrophobic microenvironments, also known as hydrophobic patches, are essential for many aspects of water-soluble proteins, from ligand or substrate binding and protein-protein interactions to proper folding after translation and aggregation during denaturation. Unlike transmembrane domains easily recognized from primary sequence, these structured, three-dimensional hydrophobic patches cannot be predicted simply using the presence of hydrophobic residues near each other in three-dimensional space. The lack of experimental strategies for directly determining their locations hinders further understanding of their structure and function.

Here, as shown in Example 5, we demonstrate that the small anionic, aromatic, and triatomic molecule N3″ (azide), is attracted to these patches, but in the presence of an oxidant, the azide loses an electron and forms a highly reactive radical that covalently attacks C—H bonds of nearby amino acids. Using two pure model proteins (BSA and lysozyme) and a cell-free lysate isolated from the model higher plant Arabidopsis thaliana, we find that radical-mediated covalent azidylation occurs within catalytical active sites and ligand binding sites. The results are consistent with a model in which the azide radical is acting as an ‘affinity reagent’ for nonaqueous three-dimensional protein microenvironments. We propose that the azide radical is a facile means of identifying hydrophobic microenvironments in soluble proteins and in addition, provides a simple new method for attaching chemical handles to proteins without the need for genetic manipulation or specialized reagents.

A critical aspect of life is the ability of large protein polymers formed from the 20 different amino acid monomers to fold into unique three-dimensional structures. Although DNA encodes all the information needed to assemble the primary sequence of these proteins, a precise understanding of how they fold remains unknown. The method described herein is specifically aimed at further understanding how water-soluble proteins create water-free hydrophobic microenvironments on their surface and buried within their three-dimensional structure. The method and observations described herein have wide potential for enabling laboratories worldwide to advance understanding of how proteins perform their critical functions.

### EXAMPLES

The following examples are included herein solely to provide a more complete disclosure of the method described and claimed herein. The examples do not limit the scope of the claims in any fashion.

### Example 1

### SUMMARY

Although hydrophobic microenvironments have been implicated in protein folding and unfolding, there are few methods that experimentally identify their precise location within a protein's 3D structure, while in solution. We report here a simple method using sodium azide and hydrogen peroxide to covalently label the side chain of amino acids located within putative hydrophobic domains of two water-soluble model proteins, BSA and lysozyme. This observation was facilitated using click chemistry to add a biotin group, enabling enrichment of the modified peptides. Mass spectrometry confirmed both the molecular structure and location of covalent azidylation in amino acid side chains of both proteins. This simple and fast Direct Azidylation and Click Capture (“DACC”) procedure provides a method for investigating the role of hydrophobic patches in the 3D structure and dynamic motion of proteins in an aqueous solution.

### BACKGROUND

The incredibly diverse ability of different sequences of amino acids to fold their amino acid side chains into precise catalytic structures, combined with high throughput genetic technologies, holds great promise for new innovations in synthetic organic chemistry (1). Critical to this work is a precise understanding of the location of a protein's amino acid side chains while in dynamic motion in an aqueous solution, rather than frozen after vitrification into a specific conformer during cryoEM or in a crystal prior to analysis by X-ray diffraction. A plethora of studies on the folding and unfolding of proteins, both in vivo and in vitro, has indicated that the shielding of short hydrophobic patches from aqueous solution is an important driving force for these dynamic processes (2, 3). To study in-solution protein dynamics, mass spectrometric based ‘footprinting’ using covalent amino acid modifying reagents such as hydroxyl radicals (“HRF,” for hydroxyl radical footprinting) has emerged as a growing field that can provide considerable insights (+). Protein footprinting can provide critical information on solvent accessible amino acid side chains, corroborate similar information derived from crystal structures (5), and with appropriately modified labeling reagents, can also access and modify the hydrophobic environments found in transmembrane domains (6). In the Example described herein, it has been found that the triatomic molecule azide is a simple, small covalent modifying reagent useful for identifying small hydrophobic patches of amino acids that are not within transmembrane domains but rather, are found in soluble proteins created within their unique three-dimensional structures.

Our model describing the location and type of attachment of the azide moiety to amino acids within proteins is consistent with the fact that the majority of azide addition chemistry reported in the literature, whether the mechanism involves the azide anion or the azido radical, occurs in organic solvents or in organic/aqueous solvent mixtures (7). Because of this, the vast majority of what is known about azidylation chemistry is inapplicable to native protein or direct biological context. That said, the azide radical's interaction with amino acids and to a lesser extent, peptides, has been studied; however, very few reports indicate that azido radicals can covalently modify free amino acids, and the evidence for this is sparse (8-12). The data obtained to date rather suggests that azido radicals abstract hydrogen from aromatic residues, creating transient sidechain radicals which can go on to create structures such as a dityrosine bridge (13). We sought to test whether a biologically applicable system could be devised to directly azidylate proteins. In order to provide definitive data on the molecular nature of this azidylation, and to identify the locations of this modification in natively folded proteins, we used “bottom-up” tandem mass spectrometric-based technology following copper click-based azide-alkyne cycloaddition.

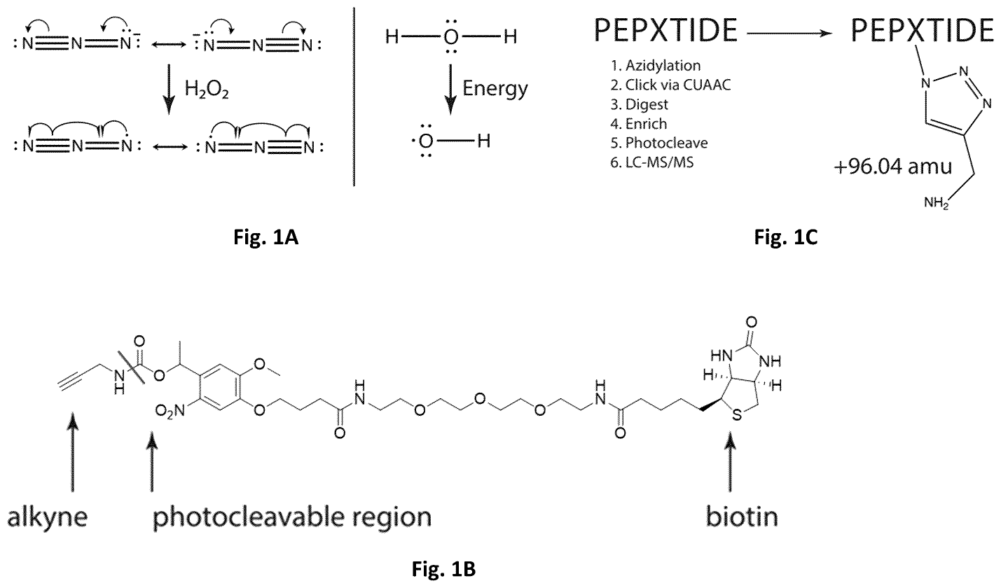

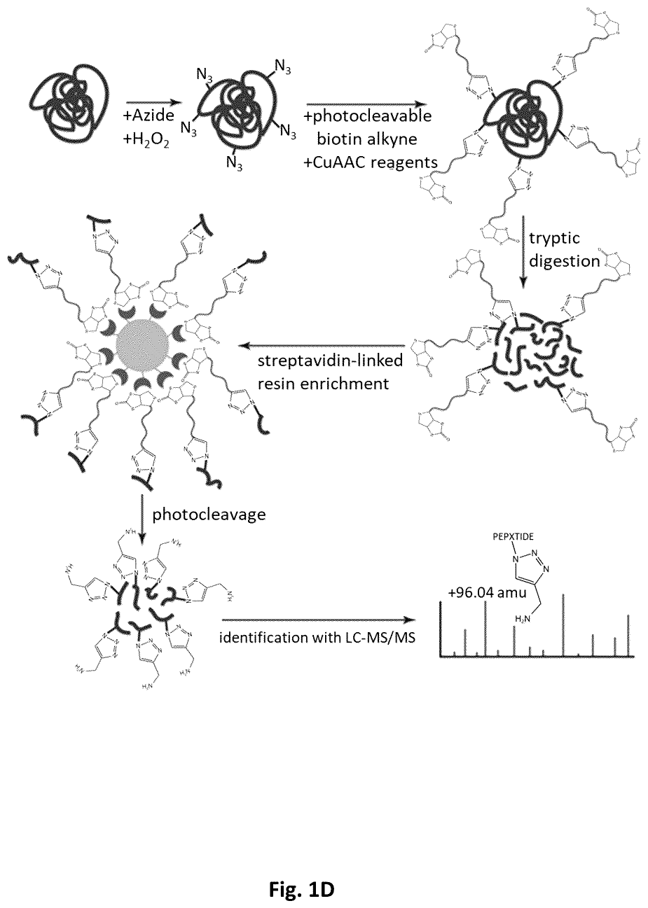

The use of hydroxyl radicals as a means of covalently labeling amino acids and nucleotides which are accessible to the solvent is a well-known method commonly called “footprinting.” There are many methods for hydroxyl radical-based protein footprinting, including synchrotron-, pulsed laser-, or plasma-induced hydroxyl radical generation from water to mixing hydrogen peroxide with protein in solution (4). It is important to note that the OH radical is a small, diatomic molecule that lacks delocalized electron density, while the azido radical is triatomic and exhibits resonance that delocalizes the singlet electron across the three nitrogen atoms (FIG. 1A), thus providing the capability of short-range pi electron attraction with aromatic molecules. We thus hypothesized that the azido radical would exhibit markedly different interaction behavior and reactivity from the hydroxyl radical, a prediction borne out by the examples described herein.

To date, the direct attachment of azido radicals to any protein has not been reported. To introduce an azide into a protein and enable azide-alkyne cycloaddition, there are currently three routes (14, 15). First, introduction of an azide-containing residue during translation; second, azide-linked site-specific chemical derivatization, such as maleimide, N-hydroxysuccinimide (NHS) ester chemistry, and N-terminal derivatization using a pyridine compound (16); or third, derivatization of primary amines to azides using diazo transfer (17, 18). The third route is limited in that at physiological pH, few amines undergo the conversion. Thus, the process takes many hours and requires specialized reagents (18). We reasoned that a method that relies upon azido free radical attack, rather than amine-induced electrophilic attack of the imidazole azide reagent, and that uses easy to obtain, safe reagents would have widespread utility for protein labelling; thus, we explored the use of azido radicals to directly label proteins

Azido radicals that interact with free amino acids are generated by irradiating water to create hydroxyl radicals, which in turn react with azide salts to produce radicalized azide (10, 11). Given this, we decided to ask whether more accessible methods of oxidation could be used to facilitate azido radical generation in a buffer system amenable to native protein in solution. Hydrogen peroxide (H₂O₂) was selected as the oxidant due to the relative ease of use, availability, and reported oxidizing capability in a protein context (19, 20). Because the efficiency of azidylation in an aqueous protein context might be low, and thus, the azidylated product may be much lower in abundance than the unmodified starting molecule, an assay was developed to detect and enrich for the modified compound via ‘clickability’ with copper-catalyzed, azide-alkyne cycloaddition (CuAAC). Using a biotinylated, photocleavable alkyne allowed capture with streptavidin followed by photorelease with light exposure (FIG. 1B). This method could also be used as an analytical tool since the clicked-on modification can be detected by both Western blotting and mass spectrometry (MS). Thus, the method we developed and disclosed herein starts with protein azidylation, which is then derivatized to a triazole using CuAAC and the alkyne, as shown in FIG. 1B. For monitoring azidylation, protein can then be run on a gel and blotted with streptavidin to detect clicked-on biotin. For a mass spectrometric based analysis, the azidylated, then biotinylated, protein may also be proteolytically digested, and the resulting modified peptides can be enriched with streptavidin resin and eluted via photocleavage to produce peptides with an expected triazole-containing mass adduct of +96.04 amu (FIGS. 1C, 1D). We call this method Direct Azidylation and Click Capture (DACC).

### Results and Discussion

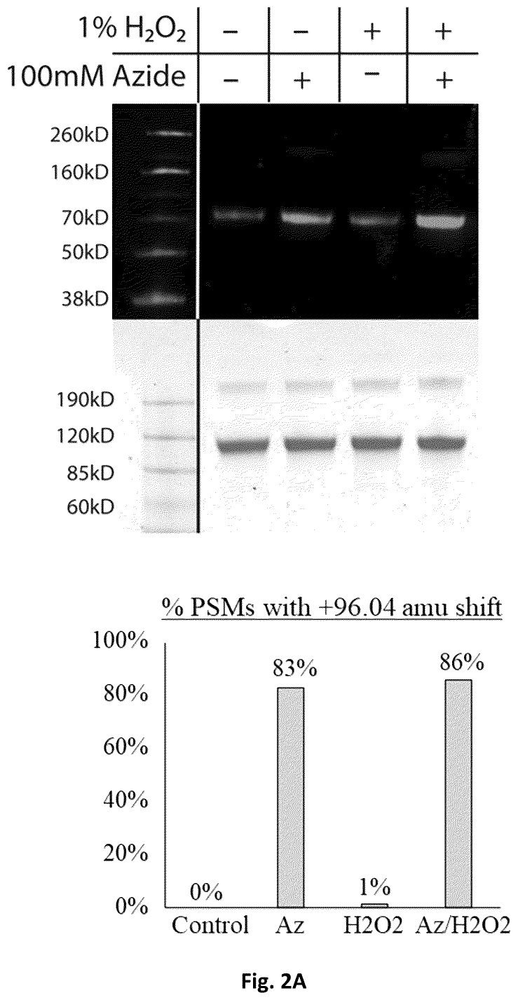

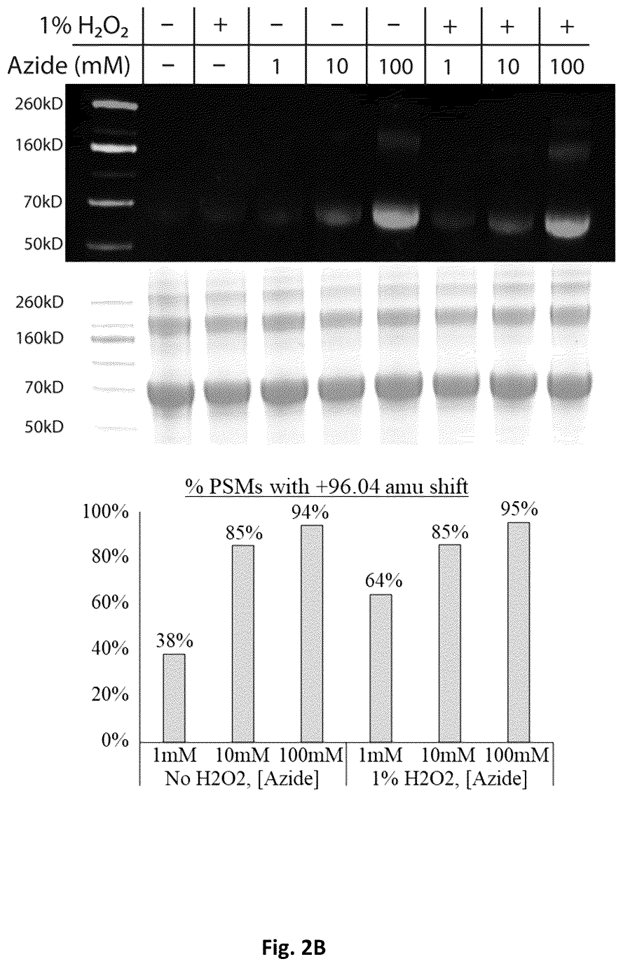

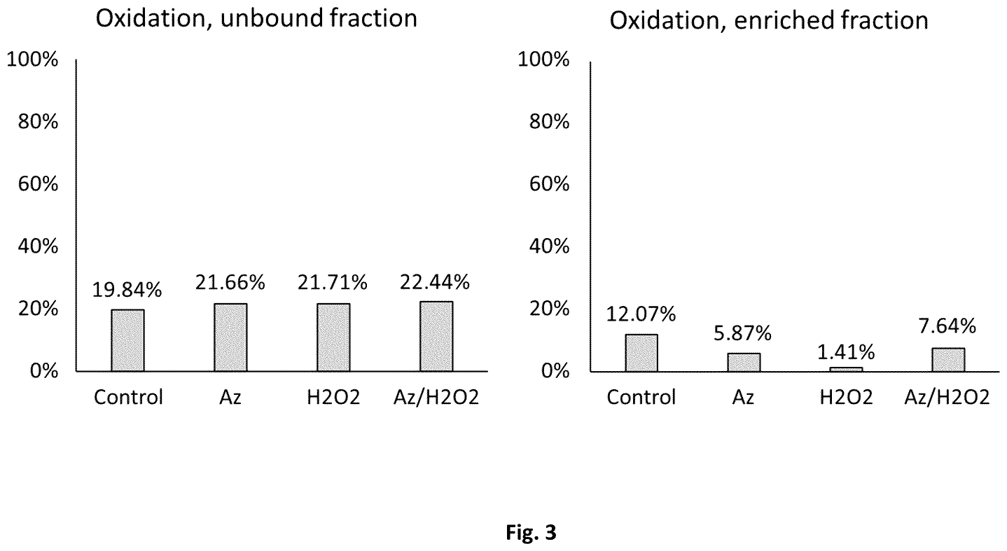

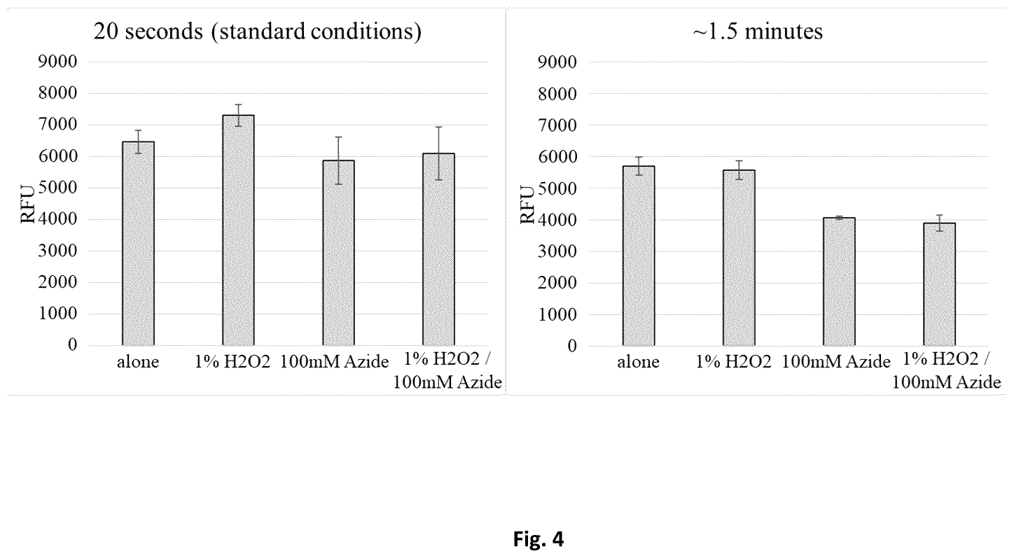

As shown in FIG. 2A, by mixing 10 μM bovine serum albumen (BSA) together with 1% H₂O₂ and 100 mM azide, azidylation of BSA was detected with both blotting and MS. Hydrogen peroxide addition alone did not lead to significant azidylation (FIG. 2A) or oxidation above control levels (FIG. 3) Unexpectedly, addition of 100 mM azide alone to BSA led to significant azidylation. To test whether this phenomenon was related to azide concentration (100 mM azide: 10 μM BSA is a molar ratio of 10,000:1) a dose-dependent experiment was performed (FIG. 2B, Table 1). In this experiment, levels of azidylation similar to that seen before were observed with the 100 mM doses of azide+/−H₂O₂. At 10 mM azide, slightly less azidylation was observed in the H₂O₂ containing samples, and at 1 mM azide, significantly less azidylation was observed when H₂O₂ wasn't added. Thus, at lower azide levels, H₂O₂ is necessary for BSA azidylation whereas at higher azide concentrations, detectable azidylation can be observed without the simultaneous addition of hydrogen peroxide. As described more fully below, we have concluded that the lack of requirement for simultaneous presence of hydrogen peroxide with azide is a unique attribute of BSA's ability to noncovalently bind azide that carries over into the click chemistry reaction mixture and which contains sufficient oxidizing capability to create amino acid reactive azido radicals.

TABLE 1
Azide dose-dependent azidylation occurs mainly on histidines or proximal to histidines on serines and is unequally distributed.
Peptide spectral match (“PSM”) counts are residue-centric, i.e., missed cleavage events that led to azidylation
on specified residues had their PSMs summed with fully cleaved peptides that contain the same azidylation site.

| | | | #PSMs | | | | | |
|---|---|---|---|---|---|---|---|---|
| | Residues | | No H₂O₂ | | | 1% H₂O₂ | | |
| | In | Site of | 1 mM | 10 mM | 100 mM | 1 mM | 10 mM | 100 mM |
| Peptides | Protein | modification | Azide | Azide | Azide | Azide | Azide | Azide |
| FKDLGEEHFK | 35-44 | H42 | — | 40 | 124 | — | 7 | 115 |
| (SEQ. ID. NO: 1) | | | | | | | | |
| DLGEEHFK | 37-44 | | | | | | | |
| (SEQ. ID. NO: 2) | | | | | | | | |
| TCVADESHAGCEK | 76-88 | H83 | — | — | 15 | — | — | 43 |
| (SEQ. ID. NO: 3) | | | | | | | | |
| SLHTLFGDELCK | 89-100 | S89 | — | 62 | 170 | — | — | 170 |
| (SEQ. ID. NO: 4) | | | | | | | | |
| QEPERNECFLSHKDDSPDLPK | 118-138 | H129 | — | — | 51 | — | — | 124 |
| (SEQ. ID. NO: 5) | | | | | | | | |
| NECFLSHKDDSPOLPK | 123-138 | | | | | | | |
| (SEQ. ID. NO: 6) | | | | | | | | |
| ECCHGOLLECADDRADLAK | 267-285 | H270 | — | — | 19 | — | — | 21 |
| (SEQ. ID. NO: 7) | | | | | | | | |
| VHKECCHGDLLECADDRADLAK | 264-285 | | — | — | 11 | — | — | 36 |
| (SEQ. ID. NO: 8) | | | | | | | | |
| SHCIAEVEK (SEQ. ID. NO: 9) | 310-318 | | | | | | | |
| SHCIAEVEKDAIPENLPPLTADFA | 310-340 | S310 | 16 | 40 | 197 | 22 | 93 | 380 |
| EDKDVCK | | | | | | | | |
| (SEQ. ID. NO: 10) | | | | | | | | |
| DOPHACYSTVFDK (SEQ. ID. | 387-399 | H390 | — | — | 42 | — | 6 | 96 |
| NO: 11) | | | | | | | | |
| EYEATLEECCAKDDPHACYSTVF | 375-399 | | | | | | | |
| DK (SEQ. ID. NO: 12) | | | | | | | | |
| LKHLVDEPONLIK | 400-412 | H403 | — | 6 | 212 | — | — | 253 |
| (SEQ. ID. NO: 13) | | | | | | | | |
| HLVDEPONLIK | 402-412 | | | | | | | |
| (SEQ. ID. NO: 14) | | | | | | | | |
| LFTFHADICTLPOTEK | 529-544 | H533 | — | — | 91 | — | — | 105 |
| (SEQ. ID. NO: 15) | | | | | | | | |

In order to provide insights into the molecular mechanisms underlying the gel-based observations, high resolution tandem mass spectrometry was used to localize azidylation sites and measure degree of azidylation (Table 1). High energy collisional dissociation (HCD) fragmentation of peptides containing the azide modification revealed that under these conditions the triazole adduct is labile, but could be retained by using electron transfer dissociation with supplemental collisional activation (EThcD), evidenced by both a richer series of fragment ions and presence of unfragmented precursor. The major sites of modification were histidine and serine, though the two azidylated serines were respectively one and two residues from a histidine. See Table 1). Consistent with stronger azide-dependent azidylation at higher azide concentrations, both total peptide spectral matches (PSMs) and PSMs per modified site increased as azide dose increased. Reproducibility between the + and − H₂O₂ samples was good; however, at 10 mM azide in the absence of H₂O₂ strong azidylation was observed on S89, which was not identified in the 10 mM azide+H₂O₂ sample. Samples supplied with 100 mM azide exhibited far more azidylation than those with lower doses—to verify that this combined treatment with H₂O₂ and azide isn't denaturing, we analyzed GFP fluorescence under identical reaction conditions and found that no fluorescence was lost, suggesting that the conditions used are nondenaturing. However, letting the reaction continue for 60 seconds beyond initial treatment led to minor loss of fluorescence in azide-containing samples, and thus it is likely that longer treatments may cause unfolding (FIG. 4).

Several other peptides were tested as well. Thus, Table 2 is a fragment ion table of peptide LKHLVDEPQNLIK (SEQ. ID. NO: 16), obtained from HCD fragmentation

TABLE 2
Fragment Ion Table for LKHLVDEPQNLIK (SEQ. ID. NO: 16)
Ion Series | Neutral Losses | Precursor Ions | Internal Fragments

| #1 | b⁺ | b²⁺ | Seq. | y⁺ | y²⁺ | #2 |
|---|---|---|---|---|---|---|
| 1 | 114.09134 | 57.54931 | L | | | 13 |
| 2 | 242.18630 | 121.59679 | K | 1529.85944 | 765.43336 | 12 |
| 3 | 475.29357 | 238.15042 | H-PC | 1401.76448 | 701.38588 | 11 |
| | | | bioti . . . | | | |
| 4 | 588.37763 | 294.69245 | L | 1168.65721 | 584.83225 | 10 |
| 5 | 687.44604 | 344.22666 | V | 1055.57315 | 528.29021 | 9 |
| 6 | 802.47299 | 401.74013 | D | 956.50474 | 478.75601 | 8 |
| 7 | 931.51558 | 466.26143 | E | 841.47779 | 421.24254 | 7 |
| 8 | 1028.56834 | 514.78781 | P | 712.43520 | 356.72124 | 6 |
| 9 | 1156.62692 | 578.81710 | Q | 615.38244 | 308.19486 | 5 |
| 10 | 1270.66985 | 635.83856 | N | 487.32386 | 244.16557 | 4 |
| 11 | 1383.75391 | 692.38059 | L | 373.28093 | 187.14410 | 3 |
| 12 | 1496.83798 | 748.92263 | I | 260.19687 | 130.60207 | 2 |
| 13 | | | K | 147.11280 | 74.06004 | 1 |

A fragment ion table of peptide LKHLVDEPQNLIK (SEQ. ID. NO: 16), obtained from EThcD fragmentation (ETD with supplemental HCD activation) is shown in Table 3.

TABLE 3
Fragment IonTable of Peptide LKHLVDEPQNLIK (SEQ. ID. NO: 16)
Ion Series | Neutral Losses | Precursor Ions | Internal Fragments

| #1 | c⁺ | c⁺H⁺ | c⁺H²⁺ | c²⁺ | c⁻H⁺ | c⁻H²⁺ | Seq. |
|---|---|---|---|---|---|---|---|
| 1 | 131.11789 | 132.12571 | 66.56650 | 66.06258 | 130.11006 | 65.55867 | L |
| 2 | 259.21285 | 260.22068 | 130.61398 | 130.11006 | 258.20503 | 129.60615 | K |
| 3 | 492.32011 | 493.32794 | 247.16761 | 246.66370 | 491.31229 | 246.15978 | H-PC biot . . . |
| 4 | 605.40418 | 606.41200 | 303.70964 | 303.20573 | 604.39635 | 302.70181 | L |
| 5 | 704.47259 | 705.48042 | 353.24385 | 352.73993 | 703.46477 | 352.23602 | V |
| 6 | 819.49954 | 820.50736 | 410.75732 | 410.25341 | 818.49171 | 409.74949 | D |
| 7 | 948.54213 | 949.54995 | 475.27861 | 474.77470 | 947.53430 | 474.27079 | E |
| 8 | 1045.59489 | 1046.60272 | 523.80500 | 523.30108 | 1044.58707 | 522.79717 | P |
| 9 | 1173.65347 | 1174.66129 | 587.83429 | 587.33037 | 1172.64564 | 586.82646 | Q |
| 10 | 1287.69640 | 1288.70422 | 644.85575 | 644.35184 | 1286.68857 | 643.84792 | N |
| 11 | 1400.78046 | 1401.78829 | 701.39778 | 700.89387 | 1399.77264 | 700.38996 | L |
| 12 | 1513.86452 | 1514.87235 | 757.93981 | 757.43590 | 1512.85670 | 756.93199 | I |
| 13 | | | | | | | K |

| | | | | | | | | #2 |
|---|---|---|---|---|---|---|---|---|
| y⁺ | y²⁺ | z⁺ | z⁺H⁺ | z⁺H²⁺ | z²⁺ | z⁻H⁺ | z⁻H²⁺ | 13 |
| 1529.85944 | 765.43336 | 1514.84854 | 1515.85637 | 758.43182 | 757.92791 | 1513.84072 | 757.42400 | 12 |
| 1401.76448 | 701.38588 | 1386.75358 | 1387.76140 | 694.38434 | 693.88043 | 1385.74575 | 693.37651 | 11 |
| 1168.65721 | 584.83225 | 1153.64632 | 1154.65414 | 577.83071 | 577.32680 | 1152.63849 | 576.82288 | 10 |
| 1055.57315 | 528.29021 | 1040.56225 | 1041.57008 | 521.28868 | 520.78476 | 1039.55443 | 520.28085 | 9 |
| 956.50474 | 478.75601 | 941.49384 | 942.50166 | 471.75447 | 471.25056 | 940.48601 | 470.74664 | 8 |
| 841.47779 | 421.24254 | 826.46689 | 827.47472 | 414.24100 | 413.73709 | 825.45907 | 413.23317 | 7 |
| 712.43520 | 356.72124 | 697.42430 | 698.43213 | 349.71970 | 349.21579 | 696.41648 | 348.71188 | 6 |
| 615.38244 | 308.19486 | 600.37154 | 601.37936 | 301.19332 | 300.68941 | 599.36371 | 300.18549 | 5 |
| 487.32386 | 244.16557 | 472.31296 | 473.32079 | 237.16403 | 236.66012 | 471.30514 | 236.15621 | 4 |
| 373.28093 | 187.14410 | 358.27003 | 359.27786 | 180.14257 | 179.63865 | 357.26221 | 179.13474 | 3 |
| 260.19687 | 130.60207 | 245.18597 | 246.19379 | 123.60054 | 123.09662 | 244.17814 | 122.59271 | 2 |
| 147.11280 | 74.06004 | 132.10191 | 133.10973 | 67.05850 | 66.55459 | 131.09408 | 66.05068 | 1 |

A fragment ion table of peptide LFTFHADICTLPDTEK (SEQ. ID. NO: 17), obtained from HCD fragmentation is shown in Table 4.

TABLE 4
Fragment Ion Table of Peptide LFTFHADICTLPDTEK (SEQ. ID. NO: 17)
Ion Series | Neutral Losses | Precursor Ions | Internal Fragments

| #1 | b⁺ | b²⁺ | Seq. | y⁺ | y²⁺ | #2 |
|---|---|---|---|---|---|---|
| 1 | 114.09134 | 57.54931 | L | | | 16 |
| 2 | 261.15975 | 131.08352 | F | 1890.88506 | 945.94617 | 15 |
| 3 | 362.20743 | 181.60735 | T | 1743.81665 | 872.41196 | 14 |
| 4 | 605.32420 | 303.16574 | F-PC bioti . . . | 1642.76897 | 821.88812 | 13 |
| 5 | 742.38311 | 371.69519 | H | 1399.65221 | 700.32974 | 12 |
| 6 | 813.42022 | 407.21375 | A | 1262.59329 | 631.80029 | 11 |
| 7 | 928.44717 | 464.72722 | D | 1191.55618 | 596.28173 | 10 |
| 8 | 1041.53123 | 521.26925 | I | 1076.52924 | 538.76826 | 9 |
| 9 | 1201.56188 | 601.28458 | C-Carbami . . . | 963.44517 | 482.22622 | 8 |
| 10 | 1302.60956 | 651.80842 | T | 803.41452 | 402.21090 | 7 |
| 11 | 1415.69362 | 708.35045 | L | 702.36685 | 351.68706 | 6 |
| 12 | 1512.74638 | 756.87683 | P | 589.28278 | 295.14503 | 5 |
| 13 | 1627.77333 | 814.39030 | D | 492.23002 | 246.61865 | 4 |
| 14 | 1728.82101 | 864.91414 | T | 377.20308 | 189.10518 | 3 |
| 15 | 1857.86360 | 929.43544 | E | 276.15540 | 138.58134 | 2 |
| 16 | | | K | 147.11280 | 74.06004 | 1 |

A fragment ion table of peptide LFTFHADICTLPDTEK (SEQ. ID. NO: 17), obtained from EThcD fragmentation (ETD with supplemental HCD activation) is shown in Table 5

TABLE 5
Fragment Ion Table of Peptide LFTFHADICTLPDTEK (SEQ. ID. NO: 17)
Ion Series | Neutral Losses | Precursor Ions | Internal Fragments

| #1 | c⁺ | c⁺H⁺ | c⁺H²⁺ | c²⁺ | c⁻H⁺ | c⁻H²⁺ | Seq. |
|---|---|---|---|---|---|---|---|
| 1 | 131.11789 | 132.12571 | 66.56650 | 66.06258 | 130.11006 | 65.55867 | L |
| 2 | 278.18630 | 279.19413 | 140.10070 | 139.59679 | 277.17848 | 139.09288 | F |
| 3 | 379.23398 | 380.24181 | 190.62454 | 190.12063 | 378.22616 | 189.61672 | T |
| 4 | 526.30240 | 527.31022 | 264.15875 | 263.65484 | 525.29457 | 263.15092 | F |
| 5 | 759.40966 | 760.41748 | 380.71238 | 380.20847 | 758.40183 | 379.70455 | H-PC bioti . . . |
| 6 | 830.44677 | 831.45460 | 416.23094 | 415.72702 | 829.43895 | 415.22311 | A |
| 7 | 945.47371 | 946.48154 | 473.74441 | 473.24050 | 944.46589 | 472.73658 | D |
| 8 | 1058.55778 | 1059.56560 | 530.28644 | 529.78253 | 1057.54995 | 529.27861 | I |
| 9 | 1218.58843 | 1219.59625 | 610.30176 | 609.79785 | 1217.58060 | 609.29394 | C-Carbami . . . |
| 10 | 1319.63611 | 1320.64393 | 660.82560 | 660.32169 | 1318.62828 | 659.81778 | T |
| 11 | 1432.72017 | 1433.72799 | 717.36764 | 716.86372 | 1431.71234 | 716.35981 | L |
| 12 | 1529.77293 | 1530.78076 | 765.89402 | 765.39010 | 1528.76511 | 764.88619 | P |
| 13 | 1644.79988 | 1645.80770 | 823.40749 | 822.90358 | 1643.79205 | 822.39966 | D |
| 14 | 1745.84755 | 1746.85538 | 873.93133 | 873.42742 | 1744.83973 | 872.92350 | T |
| 15 | 1874.89015 | 1875.89797 | 938.45262 | 937.94871 | 1873.88232 | 937.44480 | E |
| 16 | | | | | | | K |

| | | | | | | | | #2 |
|---|---|---|---|---|---|---|---|---|
| y⁺ | y²⁺ | z⁺ | z⁺H⁺ | z⁺H²⁺ | z²⁺ | z⁻H⁺ | z⁻H²⁺ | 16 |
| 1890.88506 | 945.94617 | 1875.87416 | 1876.88199 | 938.94463 | 938.44072 | 1874.86634 | 937.93681 | 15 |
| 1743.81665 | 872.41196 | 1728.80575 | 1729.81357 | 865.41043 | 864.90651 | 1727.79792 | 864.40260 | 14 |
| 1642.76897 | 821.88812 | 1627.75807 | 1628.76590 | 814.88659 | 814.38267 | 1626.75025 | 813.87876 | 13 |
| 1495.70056 | 748.35392 | 1480.68966 | 1481.69748 | 741.35238 | 740.84847 | 1479.68183 | 740.34455 | 12 |
| 1262.59329 | 631.80029 | 1247.58239 | 1248.59022 | 624.79875 | 624.29484 | 1246.57457 | 623.79092 | 11 |
| 1191.55618 | 596.28173 | 1176.54528 | 1177.55311 | 589.28019 | 588.77628 | 1175.53746 | 588.27237 | 10 |
| 1076.52924 | 538.76826 | 1061.51834 | 1062.52616 | 531.76672 | 531.26281 | 1060.51051 | 530.75889 | 9 |
| 963.44517 | 482.22622 | 948.43427 | 949.44210 | 475.22469 | 474.72708 | 947.42645 | 474.21686 | 8 |
| 803.41452 | 402.21090 | 788.40363 | 789.41145 | 395.20936 | 394.70545 | 787.39580 | 394.20154 | 7 |
| 702.36685 | 351.68706 | 687.35595 | 688.36377 | 344.68552 | 344.18161 | 686.34812 | 343.67770 | 6 |
| 589.28278 | 295.14503 | 574.27188 | 575.27971 | 288.14349 | 287.63958 | 573.26406 | 287.13567 | 5 |
| 492.23002 | 246.61685 | 477.21912 | 478.22694 | 239.61711 | 239.11320 | 476.21129 | 238.60929 | 4 |
| 377.20308 | 189.10518 | 362.19218 | 363.20000 | 182.10364 | 181.59973 | 361.18435 | 181.09581 | 3 |
| 276.15540 | 138.58134 | 261.14450 | 262.15232 | 131.57980 | 131.07589 | 260.13667 | 130.57197 | 2 |
| 147.11280 | 74.06004 | 132.10191 | 133.10973 | 67.05850 | 66.55459 | 131.09408 | 66.05068 | 1 |

A fragment ion table of peptide SLHTLFGDELCK (SEQ. ID. NO: 4), obtained from HCD fragmentation is shown in Table 6.

TABLE 6
Fragment Ion Table of Peptide SLHTLFGDELCK (SEQ. ID. NO: 4)

| | | | | Internal |
|---|---|---|---|---|
| | Ion Series | Neutral Losses | Precursor Ions | Fragments |
| #1 | b⁺ | Seq. | y+ | #2 |
| 1 | 184.08765 | S-PC bioti . . . | | 12 |
| 2 | 297.17172 | L | 1332.66165 | 11 |
| 3 | 434.23063 | H | 1219.57758 | 10 |
| 4 | 535.27831 | T | 1082.51867 | 9 |
| 5 | 648.36237 | L | 981.47099 | 8 |
| 6 | 795.43079 | F | 868.38693 | 7 |
| 7 | 852.45225 | G | 721.31852 | 6 |
| 8 | 967.47919 | D | 664.29705 | 5 |
| 9 | 1096.52179 | E | 549.27011 | 4 |
| 10 | 1209.60585 | L | 420.22752 | 3 |
| 11 | 1369.63650 | C-Carbami . . . | 307.14345 | 2 |
| 12 | | K | 147.11280 | 1 |

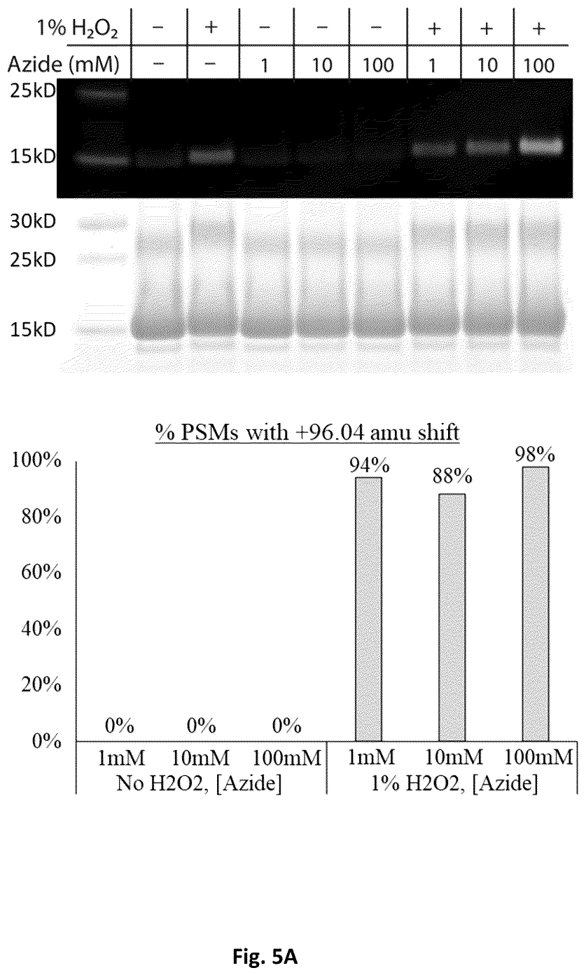

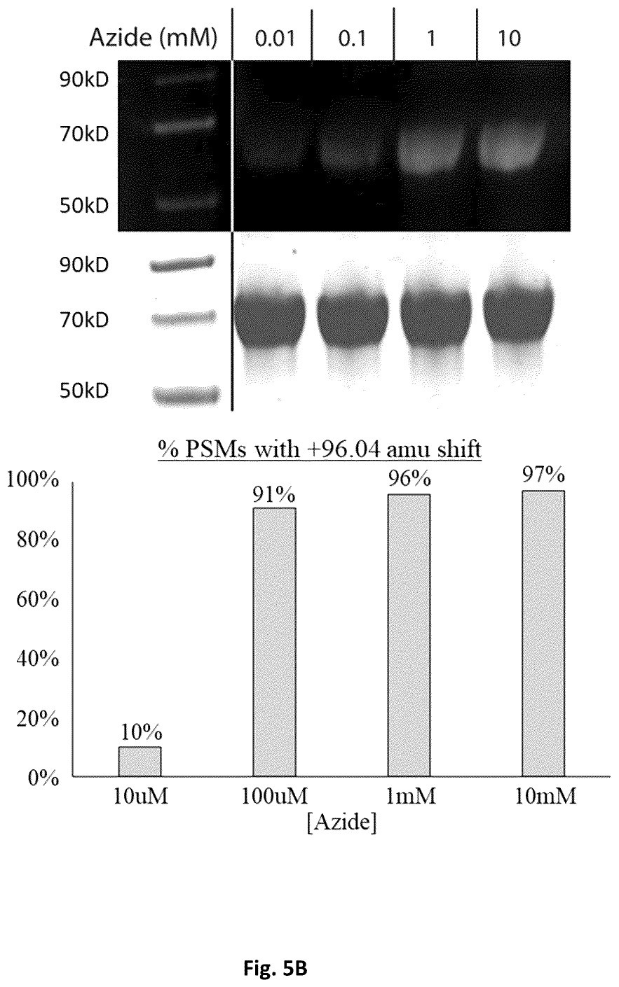

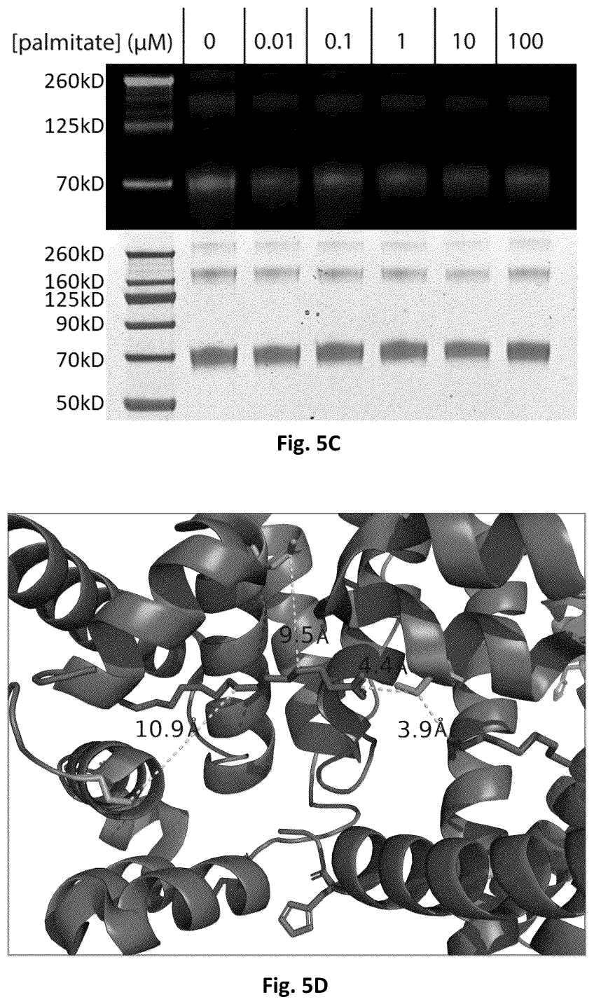

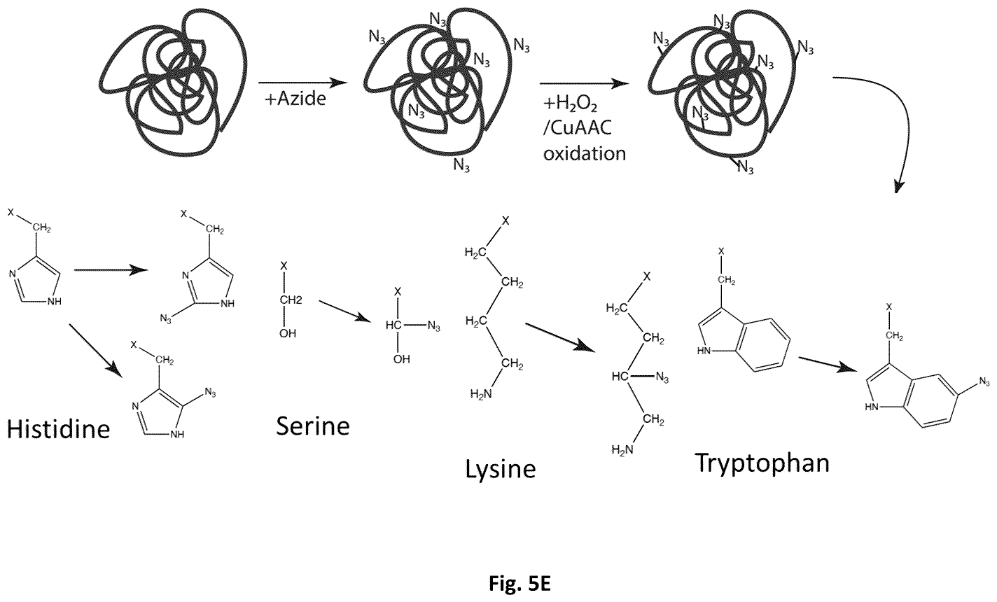

To test whether H₂O₂-independent azidylation is specific to BSA, we asked whether lysozyme, when given a dose of azide+/−H₂O₂, would display the same phenomenon. It did not. Using both Western blotting an7 MS, both H₂O₂ and azide are necessary for lysozyme azidylation (FIG. 5A, Table 7). Under these conditions, relative to BSA, lysozyme is less azidylated and we obtained fewer PSMs containing azidylated residues at similar azide/H₂O₂ doses (Table 7). By far, the most modified residue in lysozyme is tryptophan, though lysine azidylation was also seen, and no azidylation on lysozyme's single histidine was detected. Contrasted with BSA, the lysozyme data was striking and led to the hypothesis that BSA is noncovalently binding azide. This binding is tight enough so that at a minimum of 1 mM azide, enough remains through a round of trichloroacetic acid precipitation and two straight acetone washes when a buffer exchange is performed prior to CuAAC alkyne conjugation. We asked whether small amounts of added azide are sufficient for concurrent BSA azidylation and clicking. If true, this is consistent with BSA binding and carrying azide through precipitation at sufficient levels to observe the levels of azidylation seen in the absence of H₂O₂. This was indeed the case; as low as 100 μM azide added to a click reaction containing BSA that had never previously seen azide caused detectable azidylation (FIG. 5B, Table 8). MS analysis confirmed that the modification is the identical +96.04 amu mass shift observed with H₂O₂-mediated azidylation as well, suggesting that this addition proceeds via a similar mechanism as with H₂O₂. Additionally, we observed modification in regions and on residues not seen when the azidylation occurred in PBS and with H₂O₂, rather than in 4M urea and with the CuAAC reagents sodium ascorbate and cupric sulfate. For example, as with lysozyme, a modified lysine was observed, as well as a peptide containing either a modified leucine or valine (Table 3). We take this increased azidylation to be a result of urea-based alterations in the 3D structure and dynamic intramolecular motion of the protein in solution. We hypothesize that this ‘one-pot clicking’ phenomenon is due to the oxidative nature of the CuAAC reaction combined with BSA's proclivity to bind azide noncovalently. Altogether, the data suggests that BSA is increasing the local concentration of azide and after covalent attachment to the protein is supplying it to the click reaction, enabling the observed “one pot” click reaction.

TABLE 7
Lysozyme PSM count. Modification could not be localized
on peptide 46-61, and an approximately equal number of spectra
for W108 and V109 were identified.

| | | | #PSMs | | |
|---|---|---|---|---|---|
| | | | 1 | 10 | 100 |
| | | Site of | mM | mM | mM |
| Peptides | Residues | modification | Azide | Azide | Azide |
| CELAAAMKR | 6-14 | K8 | — | — | 5 |
| (SEQ. ID. NO: 18) | | | | | |
| NTDGSTDYGILQINSR | 46-61 | Not localized | — | — | 27 |
| (SEQ. ID. NO: 19) | | | | | |
| IVSDGNGMNAWVAWR | 98-112 | W108-V109* | — | — | 43 |
| (SEQ. ID. NO: 20) | | | | | |
| GTDVQAWIR | 117-125 | W123 | 16 | 113 | 244 |
| (SEQ. ID. NO: 21) | | | | | |

TABLE 8
One-pot BSA PSM count. Modifications that couldn't be localized
are noted, and generally low in abundance as evidenced by PSM counts.

| | | | #PSMs | | | |
|---|---|---|---|---|---|---|
| | | | 10 | 10 | 1 | 10 |
| | | Site of | μM | μM | mM | mM |
| Peptides | Residues | modification | Azide | Azide | Azide | Azide |
| SEIAHRFKDLGEEHFK (SEQ. ID. NO: 22) | 29-44 | H33 | — | — | — | 62 |
| FKDLGEEFHK (SEQ. ID. NO: 23) | 35-44 | H42 | — | 20 | 111 | 194 |
| GLVLIAFSQYLQQCPFDEHVK | 45-65 | Unable to | — | — | — | 58 |
| (SEQ. ID. NO: 24) | | localize | | | | |
| LVNELTEFAK (SEQ. ID. NO: 25) | 66-75 | L66/V67 | — | — | — | 21 |
| TCVADESHAGCEK (SEQ. ID. NO: 3) | 76-88 | H83 | — | — | — | 29 |
| SLHTLFGDELCK (SEQ. ID. NO: 4) | 89-100 | S89 | — | 53 | 96 | 102 |
| ETYGDMADCCEK (SEQ. ID. NO: 26) | 106-117 | Unable to | — | — | — | 6 |
| | | localize | | | | |
| QEPERNECFLSHKDDSPDLPK | 118-138 | H129 | — | — | 71 | 122 |
| (SEQ. ID. NO: 5) | | | | | | |
| AEFVEVTKLVTDLTK (SEQ. ID. NO: 27) | 249-263 | K256 | — | — | — | 13 |
| ECCHGDLLECADDRADLAK (SEQ. ID. NO: 28) | 267-285 | H270 | — | 4 | 104 | 197 |
| VHKECCHGDLLECADDRADLAK | 264-285 | | | | | |
| (SEQ. ID. NO: 8) | | | | | | |
| SCHIAEVEK (SEQ. ID. NO: 29) | 310-318 | S310 | — | 51 | 251 | 216 |
| SCHIAEVEKDAIPENLPPLTADFAEDKDVCK | 310-340 | | | | | |
| (SEQ. ID. NO: 30) | | | | | | |
| DAFLGSFLYEYSR (SEQ. ID. NO: 31) | 347-359 | Y355/357 | — | — | 11 | 56 |
| DDPHACYSTVFDK (SEQ. ID. NO: 32) | 387-399 | H390 | — | 5 | 56 | 36 |
| EYEATLEECCAKDDPHACYSTVFDK | 375-399 | | | | | |
| (SEQ. ID. NO: 12) | | | | | | |
| DDPHACYSTVFDKHKLHVDEPQNLIK | 387-412 | H403 | — | 99 | 199 | 324 |
| (SEQ. ID. NO: 33) | | | | | | |
| LKHLVDEPQNLIK (SEQ. ID. NO: 16) | 400-412 | | | | | |
| HLVDEPQNLIK (SEQ. ID. NO: 34) | 402-412 | | | | | |
| MPCTEDYLSLILNR (SEQ. ID. NO: 35) | 469-482 | Unable to | — | — | — | 4 |
| | | localize | | | | |
| AFDEKLFTFHADICTLPDTEK | 524-544 | K529 | — | — | 39 | 75 |
| (SEQ. ID. NO: 36) | | | | | | |
| LFTFHADICTLPDTEK (SEQ. ID. NO: 37) | 529-544 | H533 | — | 74 | 119 | 106 |

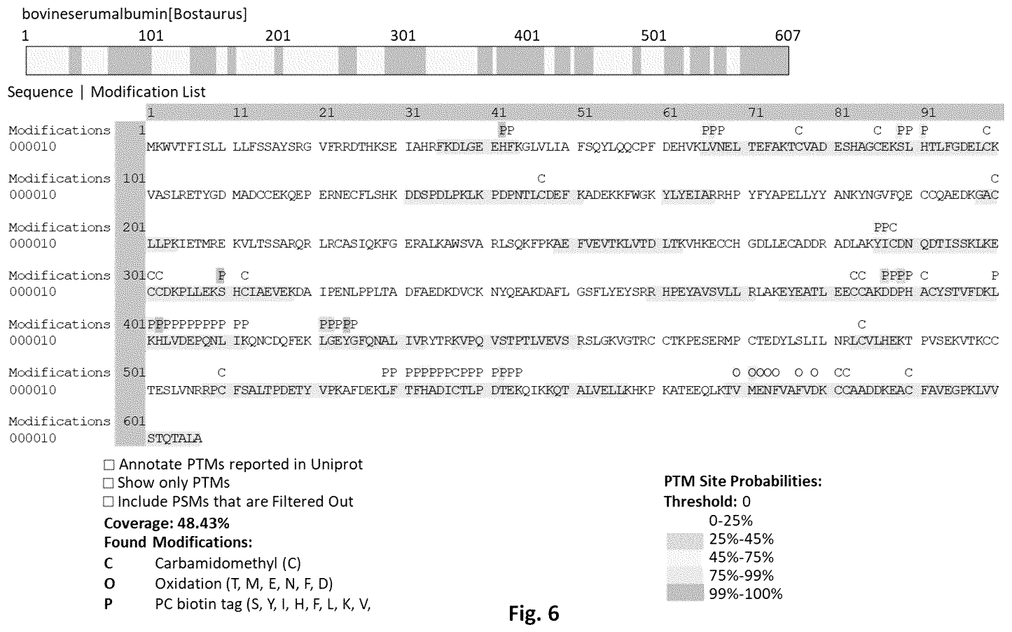

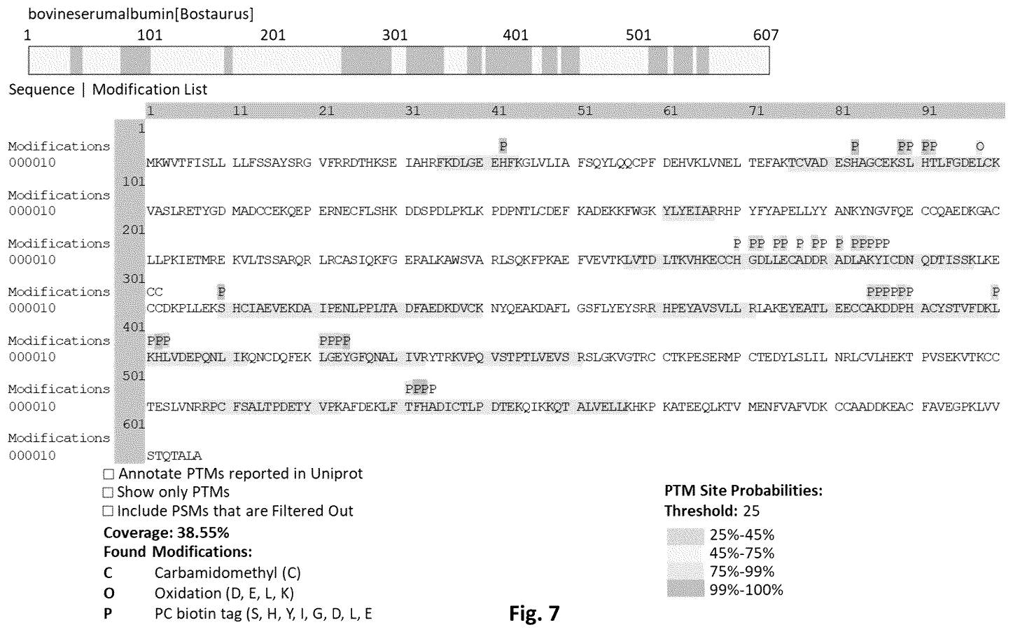

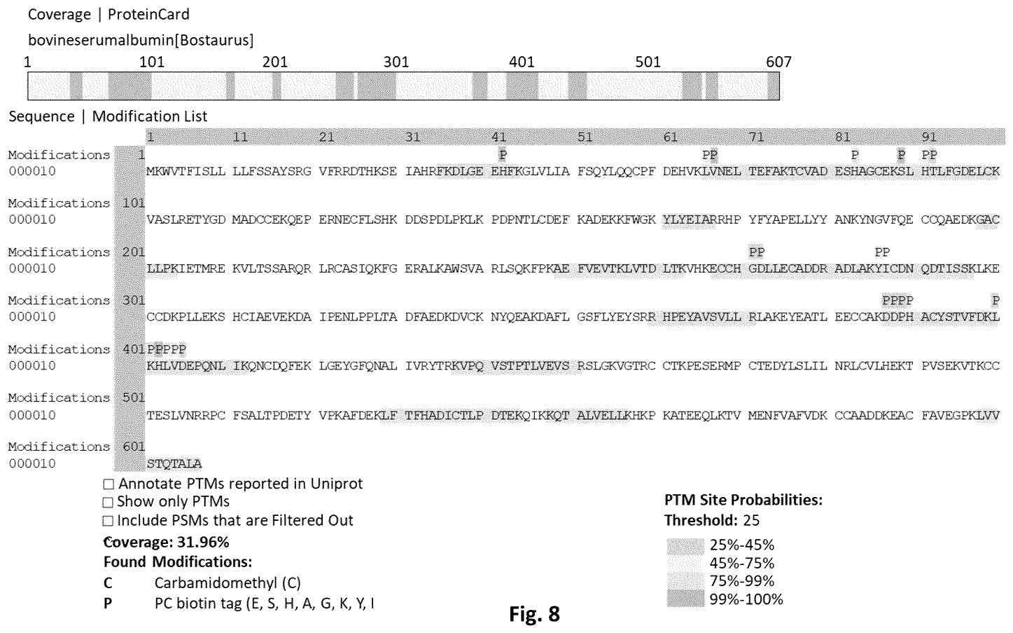

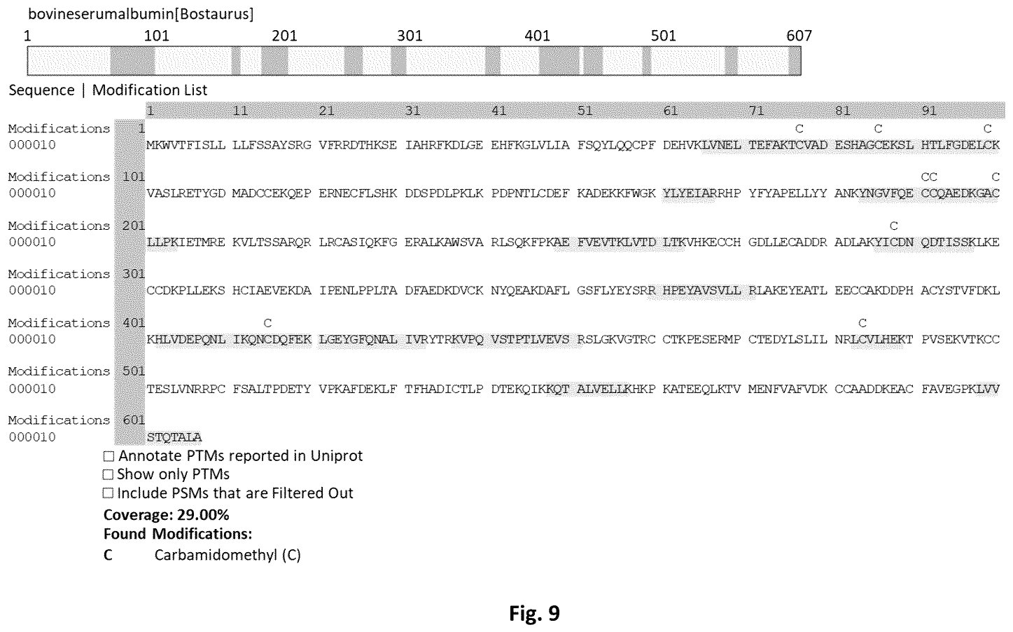

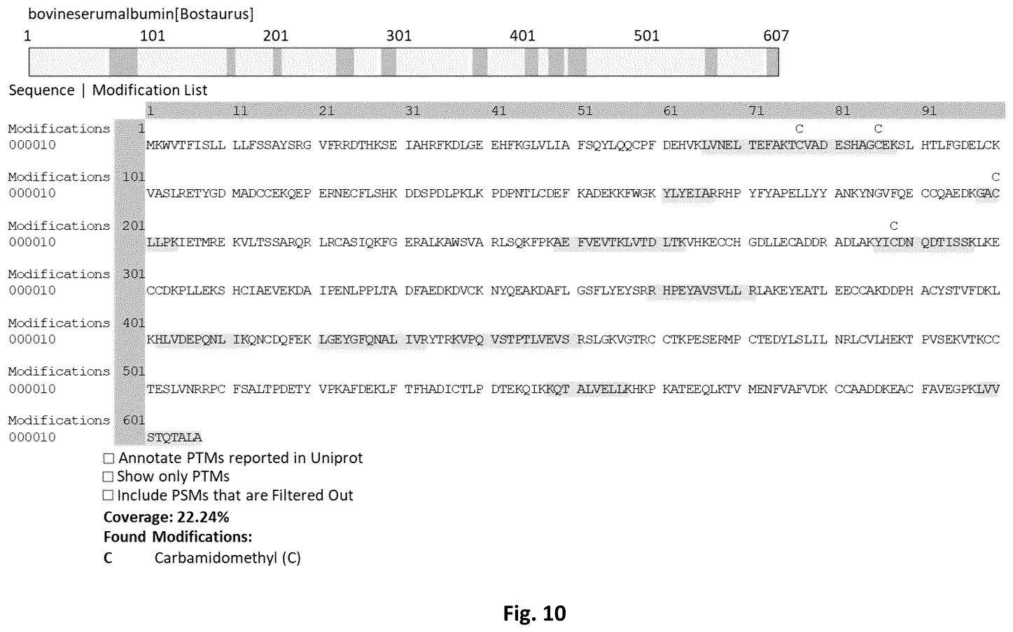

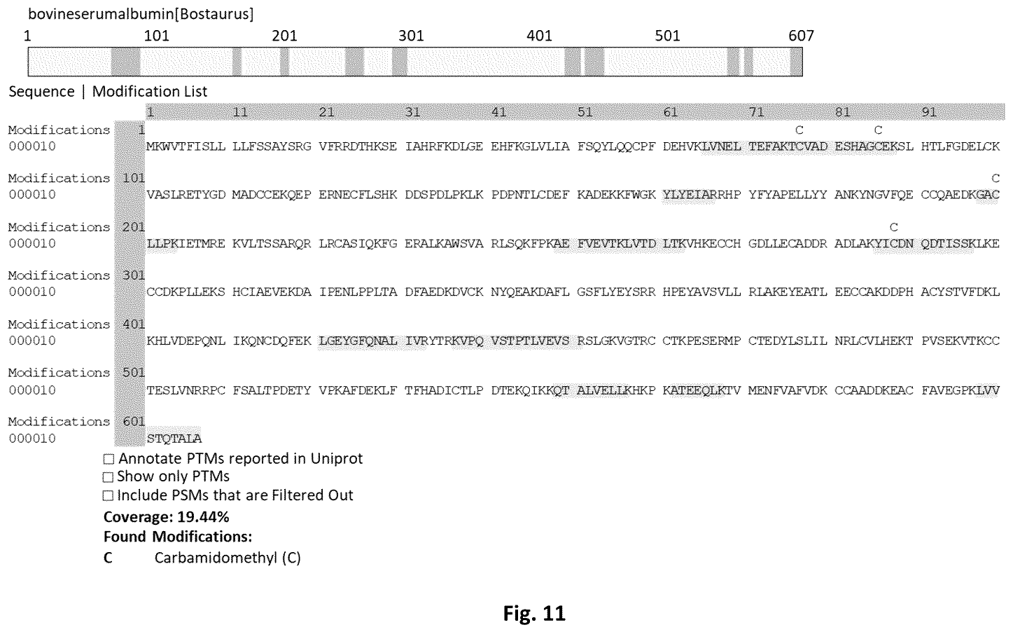

We next considered what principles could drive BSA:azide binding. The sites of BSA azidylation and solvent accessibility values derived from X-ray crystallography were not well correlated, suggesting there is some other physicochemical property or properties to account for. This interpretation was confirmed by performing a predigestion experiment, analogous to what has been done with OH radical footprinting. Previously, BSA digested into peptides demonstrated significantly more hydroxyl radical labeling in many more sites than structured, native BSA in solution, suggesting solvent accessibility is a major factor limiting OH-radical labeling (21). When an analogous experiment was done with azidylation, no modification was observed on predigested peptide samples across multiple replicates, and native BSA concurrently azidylated with the same solvents exhibited expected levels of azidylation. See FIGS. 6-11. This result is striking, particular given the opposite result with hydroxyl radicals, and is consistent with our interpretation that three-dimensional structure is necessary for a suitably hydrophobic environment to be available for covalent modification.

The BSA predigestion data led to considerations of other physical characteristics in azide binding, i.e., fundamental differences between OH and azido radicals. Because the azido radical has delocalized, resonant pi electrons, unlike the OH radical, and the most common azidylated residues are histidine in BSA and tryptophan in lysozyme, i.e. the two amino acids with a high degree of aromatic structure, we reasoned that there is resonance affinity between azide and aromatic residues. If this were the full explanation, however, then predigested BSA would exhibit a similar, or at least some, level of modification as the native protein. Investigation of BSA's biological function in the literature suggested a significant role in lipid biology-in blood, serum albumens are well known to bind fatty acids of many types with high affinity via noncovalent hydrophobic interaction (22-24). We found that palmitate added to the azidylation reaction indeed inhibited azidylation at concentrations as low as 10 nM (FIG. 5C), consistent with a model in which palmitate binds to BSA with higher affinity than azide and blocks efficient azidylation. A structure for BSA co-crystallized with palmitate does not exist; however, HSA, which exhibits good sequence and structural similarity to BSA, has been crystallized with bound palmitate (23, 24). Aligning the sequences and mapping sites that are azidylated in BSA to the HSA:palmitate structure reveals that three out of the five sites that are azidylated with 10 mM azide are within 11 Å of a pocket in HSA that binds two palmitates, and the only residue azidylated with 1 mM azide, S310, conserved in HSA as S312, is within 4.5 Å of each palmitate (data not shown).

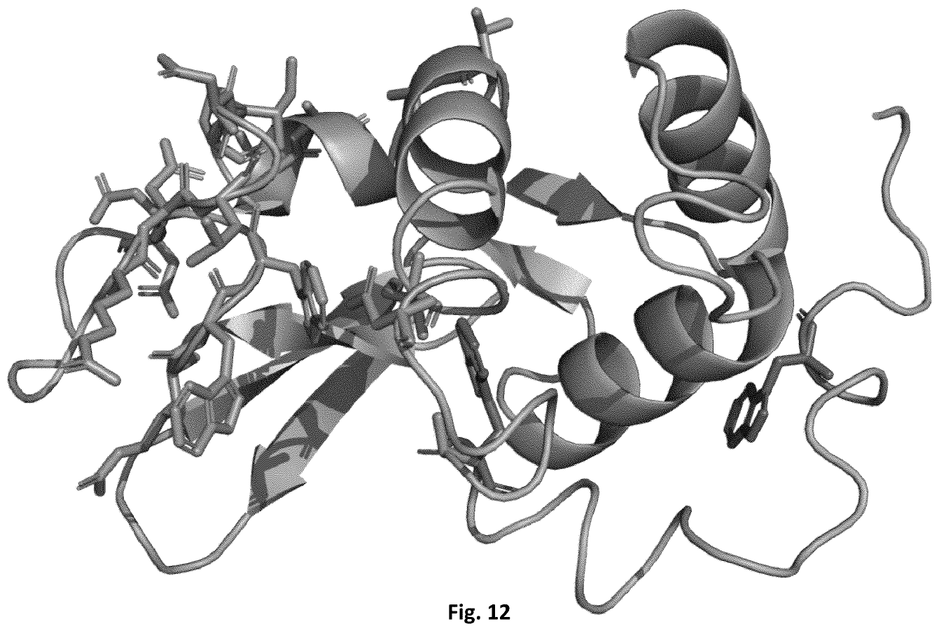

In the lysozyme dataset, there are two residues (W108 and W123) that show the greatest azide labeling. There is currently no method for predicting 3D hydrophobic microenvironments for proteins in solution, but a crystal structure-based method predicts that W108 is in direct proximity to the major hydrophobic patch and is among the most buried in this structure according to solvent accessible surface area calculations (SASA), consistent with its location within a pocket of strong hydrophobicity (FIG. 12) (25). While this software-based computational approach suggests that W123 may not be in a hydrophobic patch, given the ambiguities introduced by comparing static structures with a protein undergoing dynamic motion in solution, as well as a lack of understanding of whether solvent accessibility is by itself contradictory to azido radical labeling, our conclusion as to whether or not this second site is present in a hydrophobic microenvironment of lysozyme remains unresolved.

Finally, when azidylation is performed concurrently with CuAAC in a “one-pot” reaction, we found that many more regions of natively folded BSA were labelled, a result that was not seen when using an unfolded, previously digested BSA protein. This contrast has led us to hypothesize that as urea alters the 3D structure of a protein, the process creates more available hydrophobic pockets for azide binding. This is consistent with the idea that urea-mediated protein changes in intramolecular dynamics occur via hydrophobic solvation (26, 27). We note that BSA's interaction with 8-anilinonaphthalene-1-sulfonic acid (ANS), a valuable probe of surface exposed protein hydrophobicity, has been well-studied, though there isn't consensus on the precise sites of interaction, and the vast majority of work details protein conformational shifting, modes of ANS binding, and unspecified sites of high- and low-affinity ANS binding (28). In conclusion, these series of experiments with BSA led us to a model in which direct and covalent protein azidylation with sodium azide and a suitable oxidant, in solution, occurs primarily on aromatic residues and in hydrophobic regions only present in the folded protein. See FIG. 5E.

Though the molecular nature of the azidylation product is clear (FIG. 1C), the precise mechanism for azide addition is unknown. That said, the modification observed necessitates that only a hydrogen is lost during the reaction and replaced with an azide group, after which clicking and photocleaving forms the +96.04 amu adduct. We note that the product formed is different from that previously reported, formed by a nucleophilic displacement reaction with amines (17) and is inconsistent with addition of azide across a double bond. This leads us to hypothesize that the reaction of sodium azide with proteins, under the conditions utilized, is most likely mediated by a neutral azido free radical attack followed by hydrogen abstraction. Additionally, significant oxidation (e.g., at the highly oxidation sensitive methionine residues) above background is not observed during the reaction (FIG. 3), suggesting that under the brief reaction conditions utilized, modification is likely not proceeding by H₂O₂-mediated amino acid oxidation. Thus, we propose that the reaction proceeds via H₂O₂-induced azido radical attack of the modified residues at an available carbon, instigating a loss of hydrogen to maintain carbon's stable bonding pattern. There are, however, many unknowns remaining. For example, is the azido radical first produced in solution and then partitions into microenvironments for covalent attachment, or is the oxidant radicalizing azide after it has bound hydrophobic regions or residues? The BSA azide dosage and one-pot experiments detailed above suggest that, by separating reaction steps, noncovalent binding can precede oxidative modification and still results in the same final covalent adduct as concurrent binding/azidylation, but this is only one scenario. Future experiments performing azidylation and capture during the process of enzyme folding in vitro (e.g., with denaturants) or in vivo (e.g., during biological processes such as amyloid formation) will set the stage for exploring how the hydrophobic character of the azide molecule can be exploited for a more comprehensive understanding of protein structure in aqueous solutions.

Prior to our work, to attach azide moieties to proteins, it was necessary to use noncanonical azide-containing amino acids during translation, preexisting conjugation chemistry (e.g., NHS ester, maleimide, etc.) containing an azide moiety, or to perform diazo transfer reactions (14, 15, 17, 18). The method described herein, DACC, enables rapid, facile direct attachment of the simple triatomic molecule, azide, directly to protein in physiological buffer and at physiological pH, which can then be conjugated to secondary alkyne molecules for many additional uses, via the CuAAC click reaction. DACC demonstrates that both soluble model proteins, lysozyme and BSA, can be quickly azidylated and further derivatized, whereas BSA alone binds azide via a combination of noncovalent aromatic residue coordination, three-dimensional hydrophobic interactions, and free radical mediated covalent bond formation. Beyond assessing three-dimensional protein hydrophobicity, DACC also presents a means for facile, click-based derivatization of proteins in vitro as a new way to enable chemical biology and synthetic protein chemistry experiments.

### Materials/Methods

### Materials

Unless otherwise noted, materials were acquired commercially from Millipore Sigma

### General:

Bovine serum albumin
Lysozyme
Sodium Azide
Hydrogen peroxide, 30%
Phosphate-buffered saline
Methanol
Chloroform
Trichloroacetic acid
Zeba spin desalting columns (Thermo Fisher Scientific)
Sodium Ascorbate
Cupric Sulfate
Palmitic acid
   Click Reagents (Purchased Commercially from Click Chemistry Tools, Scottsdale, Arizona, USA):
Photocleavable biotin alkyne
Photocleavable biotin DBCO
AZDye 680 Alkyne
AZDye 680 DBCO
AZDye 800 DBCO
tris-hydroxypropyltriazolylmethylamine (THPTA)

### Gel Electrophoresis/Western Blotting:

Bolt 4-12% Bis-tris polyacrylamide gels (Invitrogen, Waltham, Massachusetts, USA)
Immobilon-FL Transfer Membrane, PVDF, pore size 0.45 μm
IRDye 800CW Streptavidin (Li-COR Biosciences, Lincoln, Nebraska, USA)
EZView Red Streptavidin Affinity Gel (Sigma-Aldrich)

### Mass Spectrometry Processing:

Urea
Dithiothreitol
Iodoacetamide
Trypsin/Lys-C(Promega, Fitchburb, Wisconsin, USA)
Ammonium Bicarbonate
Acetonitrile, MS-grade
Formic acid, MS-grade
0.1% formic acid, MS-grade
Pierce 10 uL C18 tips (Thermo Scientific, Waltham, Massachusetts USA)
OMIX C18 100 uL tips (Agilent Technologies, Santa Clara, CA 95051 USA)

### Protein Azidylation and Click Chemistry:

In final reaction volumes of 1 mL, BSA or lysozyme solubilized into PBS were mixed with 10% H₂O₂, 1M sodium azide, and PBS for final concentrations of 1% H₂O₂, between 1 mM and 100 mM sodium azide, and 10 μM protein. The azidylation reaction is performed as follows: Protein and azide are added together in PBS with final volume of 900 μL and are pipetted or vortexed very gently (setting 2-3) to mix to homogeneity. 100 μL of 1% H₂O₂ is added, reaction is gently vortexed (setting 2-3) or pipetted to mix for 5 seconds, then allowed to rest at room temperature for 15 seconds. 100 μL of saturated, cold trichloroacetic acid (TCA) is added to precipitate protein, sample is vortexed to mix, and put on ice for 15 minutes. Samples were spun at full speed in a microcentrifuge for 10 minutes at 4° C., and azide-containing supernatant was pipetted off and safely disposed of. Protein pellets were broken up and suspended into 500 μL ice cold acetone and put on ice for 5 minutes. Samples were spun at full speed in a microcentrifuge for 10 minutes at 4° C., and supernatant was discarded. Acetone wash was repeated a second time, and protein pellets were air dried for 5 minutes. Pellets were resolubilized into 8M urea in 50 mM ammonium bicarbonate and then diluted to 4M urea with 50 mM ammonium bicarbonate. To perform the click reaction, the following reagents were added, in the following order, for the following final concentrations: 4 μL 100 mM tris hydroxypropyltriazolylmethylamine (THPTA) in water, final concentration 3.5 mM, 4 μL, 20 mM cupric sulfate in water, final concentration 708 μM, 4 μL 300 mM sodium ascorbate in water, final concentration 10.6 mM, and 1 μL 5 mM photocleavable biotin alkyne in dimethyl sulfoxide, final concentration 44 μM. Reagents were pipetted to mix to homogeneity and reacted at room temperature for 20 minutes in the dark. From this reaction, 15 μL of the lysozyme reaction were taken for SDS-PAGE analysis and 0.5 μL of the BSA reaction were taken for SDS-Page analysis, below. The remainder of each was used for digestion and further processing, below.

### SDS-PAGE and Western Blotting Analysis:

Samples for SDS-PAGE analysis were evenly split and concurrently run on two gels using a Novex Bolt mini gel tank. One gel was stained with Coomassie for assessing protein amount, and one was prepped with membrane transfer buffer and transferred to a membrane for 10 minutes using an Invitrogen iBlot system. Membranes was air-dried to completion, then washed with methanol, water, and blocked with PBS Intercept blocking buffer for 1 hour. 1 μL of IRDye 800CW Streptavidin was added and reacted in the cold and dark overnight. Membranes were washed with PBS-T four times, then with PBS, then allowed to air dry in the dark. Membranes were imaged using a Li-Cor Odyssey scanner and Li-Cor Image Studio v 3.1, and Coomassie gels were imaged using an Epson V850 Pro scanner and Silverfast 8 software.

### Protein Digestion and Enrichment:

Click reaction volume not used for SDS-PAGE was diluted to 1M urea with 50 mM ammonium bicarbonate, and cold, saturated TCA was added to a final concentration of ˜10%. Samples were vortexed to mix, and put on ice for 15 minutes. Samples were spun at full speed in a microcentrifuge for 10 minutes at 4° C., and supernatant was pipetted off and safely disposed of. Protein pellets were broken up and suspended into 500 μL ice cold acetone and put on ice for 5 minutes. Samples were spun at full speed in a microcentrifuge for 10 minutes at 4° C., and supernatant was discarded. Pellets were air dried, resolubilized into 8M urea in 50 mM ammonium bicarbonate and diluted to 4 M urea with 50 mM ammonium bicarbonate. Dithiothreitol was added to a final concentration of 2 mM and samples were reduced at 42° C. for 40 minutes. Samples were cooled to room temperature, iodoacetamide was added to a final concentration of 5 mM, and samples were alkylated at room temperature in the dark for 40 minutes. A second aliquot of DTT was added to a final concentration of 4 mM and alkylation was quenched for 5 minutes at room temperature. Samples were diluted to 1M urea with 50 mM ammonium bicarbonate, and a 1:1 mix of trypsin/lys-C was added to a ratio of 100:1 protein:protease. Samples were digested for 12 hours at 37° C., then held at 2° C. thereafter until enrichment.

For enrichment, 50 μL of EZView Red Streptavidin affinity gel slurry was used per sample. Enrichment media was equilibrated with two washes of 1 mL 50 mM ammonium bicarbonate followed by two 1 mL washes with 1M urea in 50 mM ammonium bicarbonate. For equilibration and washes, media was spun at 8.2×G for 30 seconds to pellet gel. After equilibration, resin was moved to ice and protein digests (˜400 μL) were added directly to gel pellet from 2° C. incubation post-digest. Samples were incubated with end over end mixing for 2 hours at 4° C. in the dark. Unbound fractions were saved, and enrichment media was washed twice with 1 M urea in 50 mM ammonium bicarbonate and twice with 50 mM ammonium bicarbonate. 100 μL 18 MW water was added to resin, resuspended, and samples were moved to a 250 μL thin-walled clear PCR tube. Samples were laid on their side 5 cm from a 365 nM light source on an ice pack for 30 minutes to photocleave using a Stratagene UV stratalinker 1800, and supernatant was moved to a fresh 1.5 mL low-bind microcentrifuge tube. Unbound fractions were acidified with neat formic acid to 1% and cleaned up using Agilent OMIX tips according to manufacturer's protocol, and photocleaved peptide samples were acidified with neat formic acid to 1% and cleaned up with Thermo Scientific Pierce 10 μL C18 tips according to manufacturer's protocol. Samples were dried down to completion in a vacuum centrifuge following C18 cleanup.

### One-Pot Click Chemistry:

100 μM BSA was solubilized into 8M urea/50 mM ammonium bicarbonate and diluted to 50 μM with 50 mM ammonium bicarbonate. 1 μL of serial sodium azide dilutions in PBS were added to 100 μL of 50 μM BSA to achieve the final concentrations used in the experiment. CuAAC click was performed as above, with 2 μL of PC biotin alkyne in DMSO used instead of 1, final concentration 88 μM. Samples were reacted as above, 0.5 μL was sampled for Coomassie gel and blot, and the remainder was processed as above for mass spectrometry analysis.

### Palmitate Competition:

BSA was prepared as in “Protein Azidylation” section. Palmitic acid was solubilized into chloroform and 1 μL of 100× concentrated palmitic acid (or chloroform, for control) was added to 15 μM BSA in 79 μL PBS. Samples were vortexed gently to homegenity and let sit at room temp for 10 minutes. Azidylation and TCA precipitation then proceeded as in “Protein Azidylation” section with 100 mM azide and 1% H₂O₂. For the CuAAC click reaction, all volumes and reagent concentrations were the same, but the fluorescent AZDye 680 alkyne was used instead of the previous alkyne. 0.5 μL of the reaction was loaded onto an SDS-PAGE gel. Gel was first fluorescently imaged and then Coomassie-stained and imaged.

### Mass Spectrometry:

Photocleaved samples were resuspended into 10 μL Optima LC/MS-grade 0.1% formic acid, and unbound samples were resuspended into 80 μL Optima LC/MS-grade 0.1% formic acid. For both sample types, a Thermo Scientific Dionex UltiMate 3000 was used to inject peptides onto a 50 cm, 2 μM, 200 Å pore size bead-containing Thermo Scientific PepMap RSLC C18 column in a Thermo Scientific EasySpray Source. Peptides were sprayed with 1900V into a Thermo Scientific Orbitrap Fusion Lumos Tribrid Mass Spectrometer. Mobile phase A was 0.1% formic acid, and mobile phase B was 80% acetonitrile/0.1% formic acid, and flow rate was 300 nL/min. Different methods of analysis and chromatography were used; 1 μL of unbound samples were injected, and 2-3 μL of photocleaved samples were injected.

Unbound samples were analyzed using the following LC gradient: background running and equilibration buffer was 2% B, a gradient from 5% B to 37.5% B over 23 minutes, followed by a fast ramp gradient from 37.5% B to 95% B for 3 minutes, flushing at 95% B for 5 minutes, and re-equilibrated to 2% B for 10 minutes. Mass spectrometry acquisition was as follows: MS1 scans were acquired in the Orbitrap Mass analyzer with a resolution of 120K, scan range of 350-1600 m/z, AGC target of 1e6, max inject time of 50 ms, and in profile mode. For MS2 acquisition, a cycle time method with 1 s fixed spacing between MS1 scans was used, monoisotopic peak selection was used in peptide mode, charge states of 2-4 were selected, and dynamic exclusion was used with settings of n=1 for 10 second exclusion. MS2 spectra were acquired in the linear ion trap with quadrupole isolation window set to 0.7 m/z, scan range set to auto, a fixed HCD energy of 30% for fragmentation, scan rate set to turbo, an AGC target of 3e5, a max inject time of 25 ms, and in centroid mode.

Photocleaved samples were analyzed using the following LC gradient: background running and equilibration buffer was 2% B, a gradient from 5% B to 37.5% B over 38 minutes, followed by a fast ramp to 37.5% B to 95% B, flushing at 95% B for 5 minutes, and re-equilibrated to 2% B for 10 minutes. Mass spectrometry acquisition was as follows: MS1 scans were acquired in the Orbitrap Mass analyzer with a resolution of 120K, scan range of 350-1600 m/z, AGC target of 1e6, max inject time of 50 ms, and in profile mode. For MS2 acquisition, a cycle time method with Is fixed spacing between MS1 scans was used, monoisotopic peak selection was used in peptide mode, charge states of 2-7 were selected for fragmentation. MS2 spectra were acquired in the Orbitrap with quadrupole isolation window set to 0.7 m/z, scan range set to auto, an AGC target of 1.5e5, a max inject time of 54 ms, and in centroid mode. Either fixed HCD collision energy of 30% or, most of the time, as specified in the main text, electron transfer dissociation with supplemental HCD activation energy was used as the fragmentation technique. Charge-dependent calibration parameters were used for ETD reaction time, and MS2 spectra were acquired in centroid mode. Dynamic exclusion was not used for photocleaved samples, to get more MS2 for localization on modified peptides.

### Database Searching:

Raw data was searched in Proteome Discoverer v 2.4. All data was searched against a database containing the sequence of BSA and common and lab-specific contaminants (207 proteins total)

Unbound data was searched with full tryptic cleavage specified with up to 2 missed cleavages and a minimum peptide length of 6. MS1 mass tolerance was set to 10 ppm, and MS2 mass tolerance was set to 0.6 Da. B- and y-ions were considered for matching, and both oxidation and the PC biotin cleaved tag adduct (+96.04) were set as dynamic modifications on all residues. Carbamidomethylation was set as a dynamic modification on cysteine residues. A concatenated target/decoy selection strategy was used with a strict FDR of 1%.

Photocleaved data was searched with full tryptic cleavage specified with up to 2 missed cleavage events. MS1 mass tolerance was set to 10 ppm, and MS2 mass tolerance was set to 0.1 Da. B-, y-, c-, and z-ions were considered for matching, and both oxidation and the PC biotin cleaved tag adduct (+96.04) were set as dynamic modifications on all residues except cysteine. Carbamidomethylation was set as static on cysteine residues. A concatenated target/decoy selection strategy was used with a strict FDR of 1%.

All of the spectra mentioned and used in the main text of the paper have been manually examined for both confidence and localization.

eGFP Fluorescence Assay:

Triplicate samples were prepared using eGFP in PBS. Per sample, 500 ng of eGFP was used, and total reaction volumes were 100 μL. All reagents except eGFP were added together, pipetted to mix, and then eGFP was added. Plate was sent into a Tecan-brand shaker for 5 seconds of orbital shaking followed by 15 seconds of resting (as close to our benchtop conditions as possible) were performed before measuring fluorescence. Fluorescence was measured with the following settings: excitation/emission of 485 nm/535 nm, a gain of 80, 3 flashes, 0 lag time, 40 us integration time, and at room temperature. After shaking and measuring the first time, fluorescence was measure at ˜t=1.5 min without shaking and with the above conditions.

BSA Native vs. Digest Experiment:

Native/intact BSA treatment was performed as specified in main methods sections. Specifically, for the native vs. digest comparison, the same reagents were used for azidylation, CuAAC clicking, reduction, alkylation, digestion, enrichment, and solid phase extraction of enriched, photocleaved fractions. In order to azidylate peptides, BSA was first digested to peptides using the protocol in main methods section. BSA digests here were desalted and concentrated using Waters 1 cc sep-pak solid phase columns, after which they were dried to completion by vacuum centrifugation. Peptides were resuspended in PBS such that final concentration was 100 μM and equivalent reaction conditions as to native protein were used. Peptide azidylation was performed as specified in main text protocol section. Per replicate, following azidylation, samples were acidified to 1% final concentration of formic acid using neat formic acid, gently vortexed, and immediately run twice through a pre-equilibrated Waters 1 cc sep-pak to desalt and concentrate peptide mixture, and were then dried to completion by vacuum centrifugation. Peptides were resuspended into 8M urea/50 mM ammonium bicarbonate, diluted to 4M urea, and click was performed as described in main text section. Following click, peptides were again cleaned up using Waters 1 cc sep-pak solid phase columns and dried to completion by vacuum centrifugation. Peptide mixtures were resuspened into 1M urea/50 mM ammonium bicarbonate and enriched, cleaned up, and analyzed as detailed in main text protocols.

### Example 2

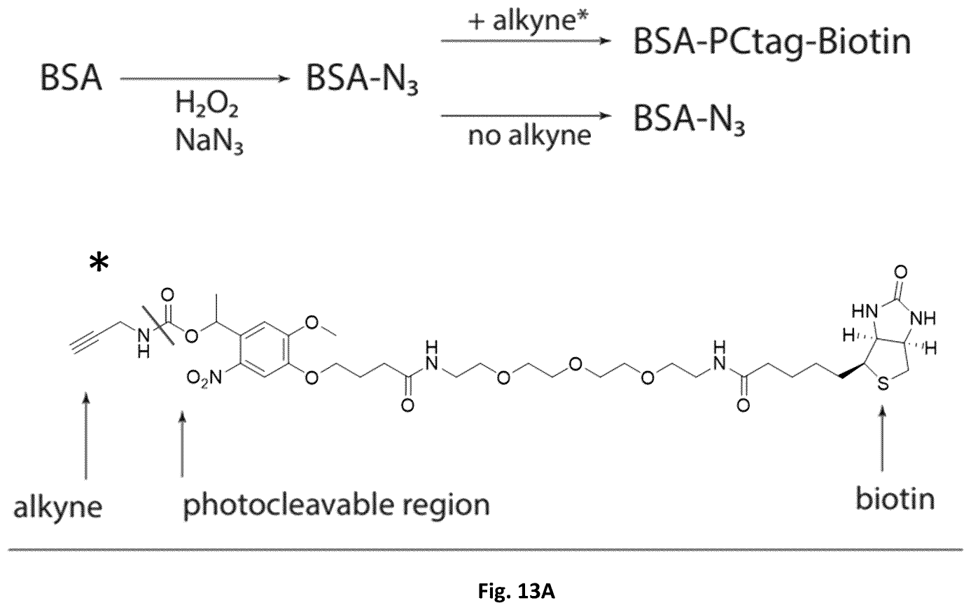

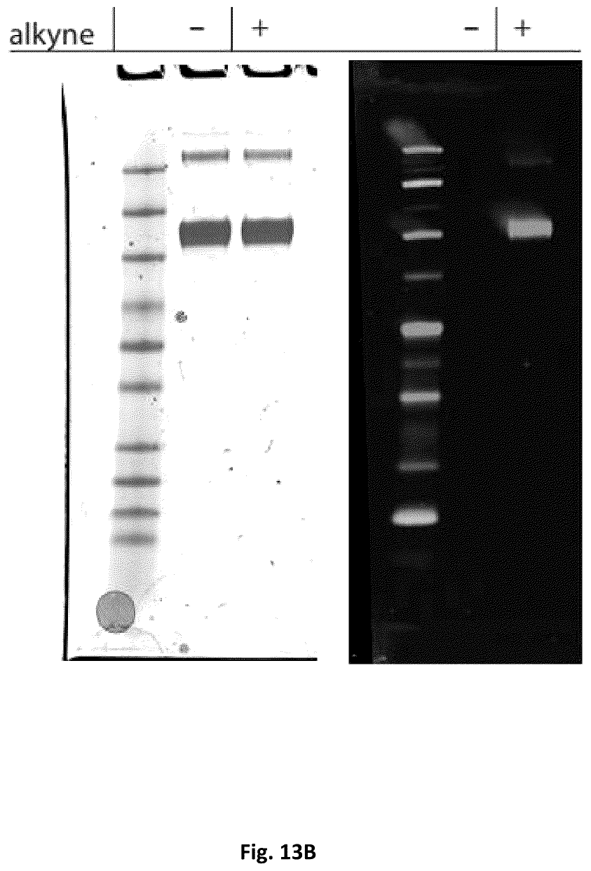

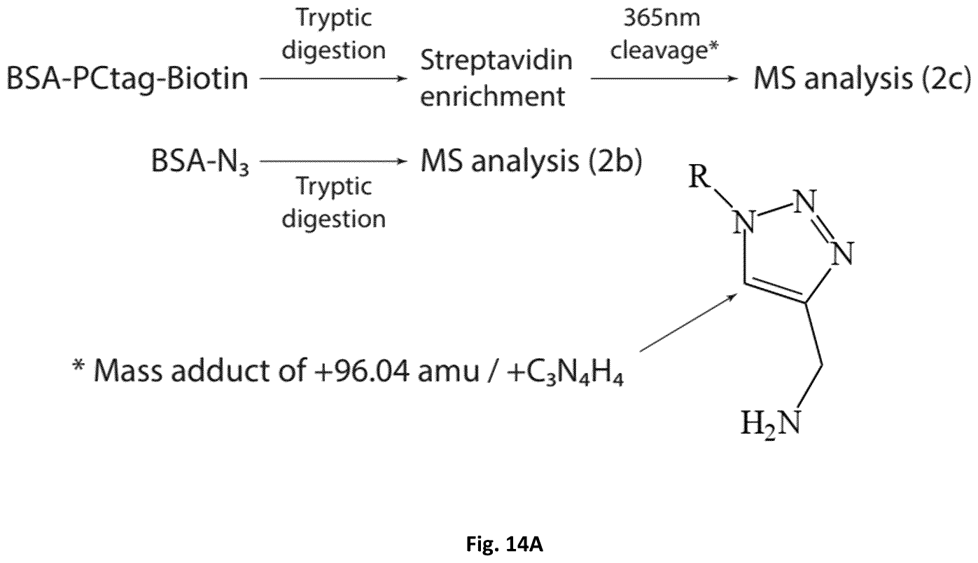

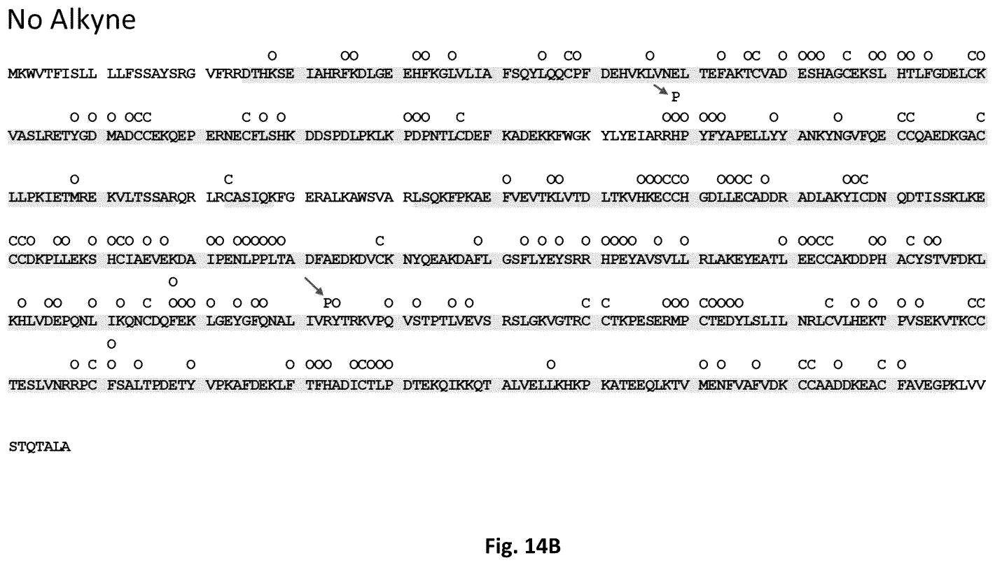

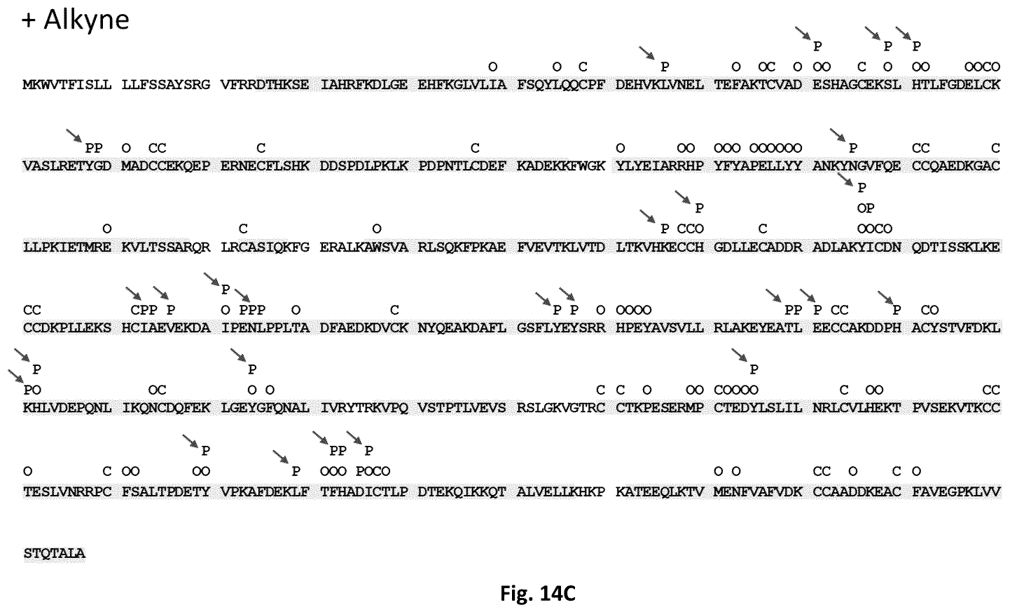

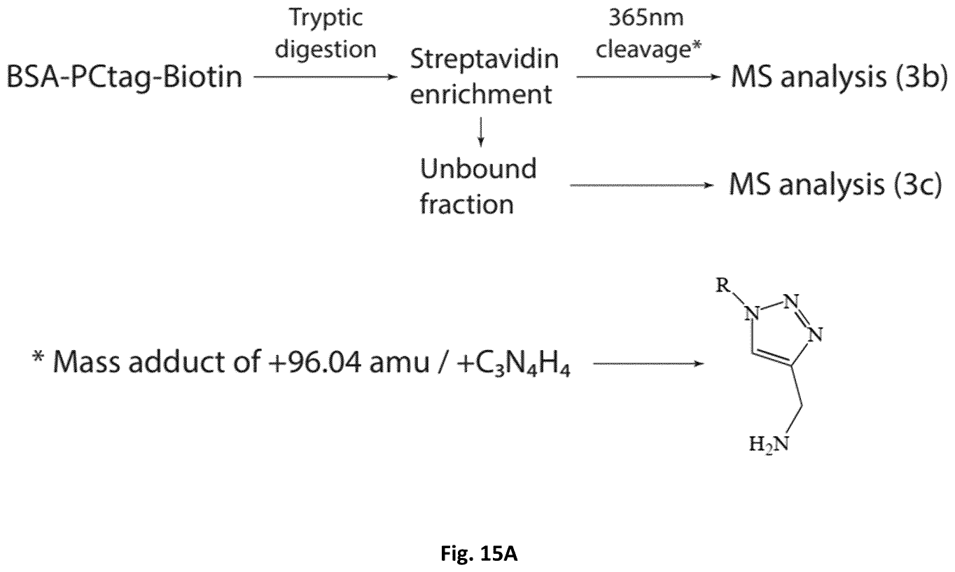

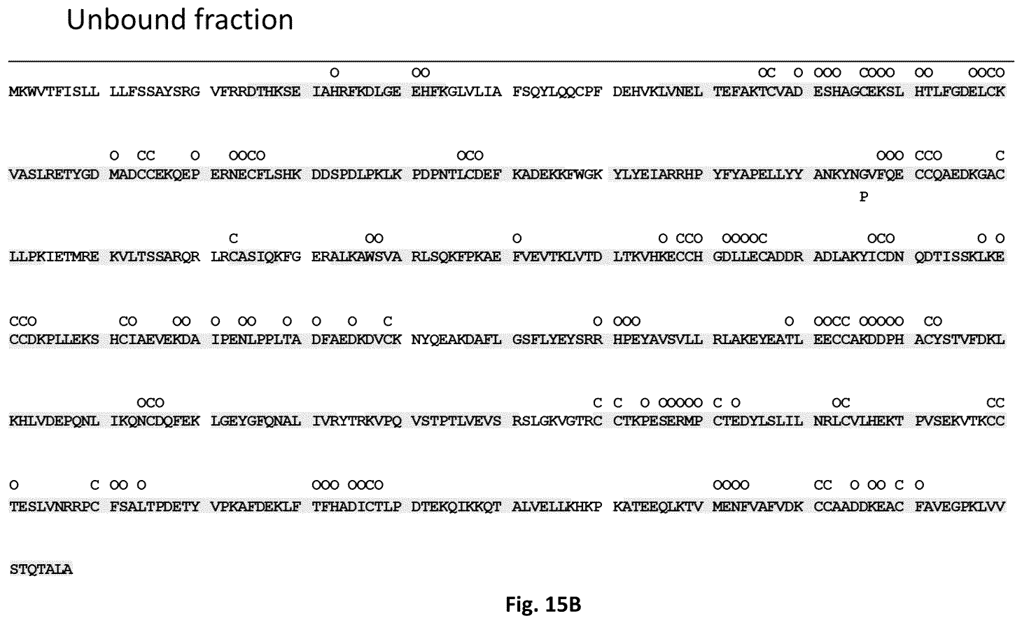

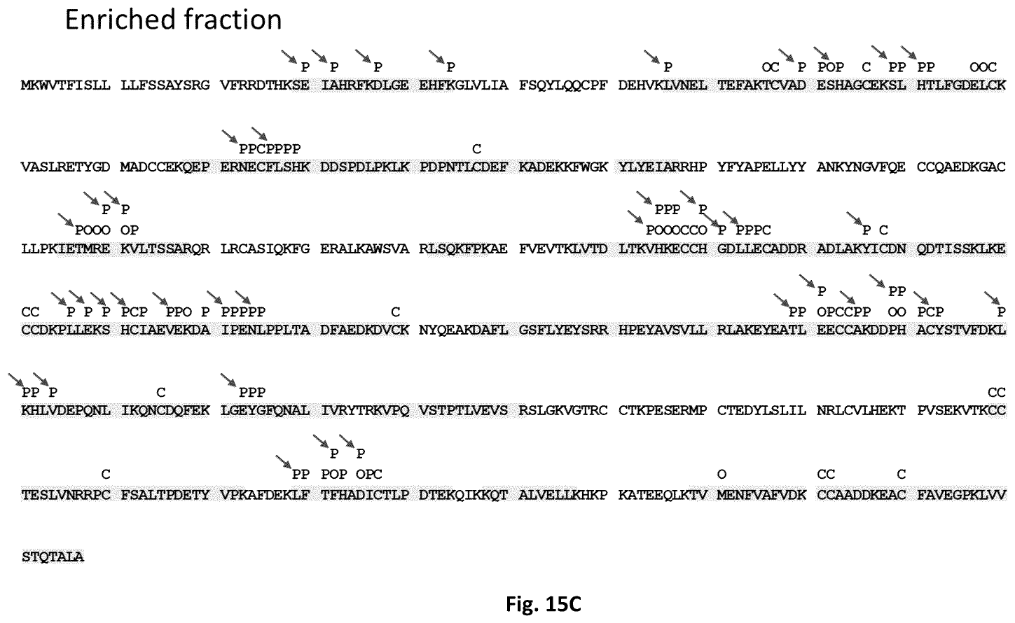

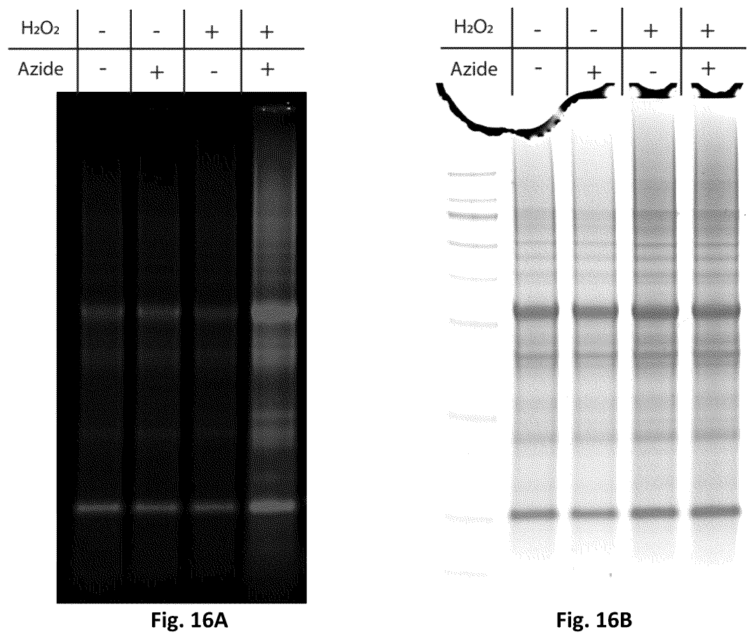

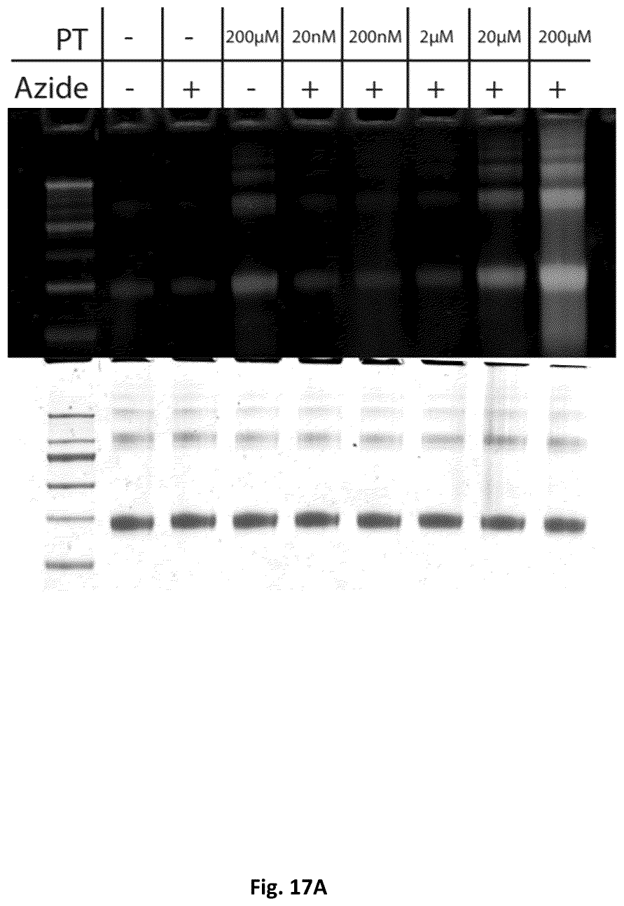

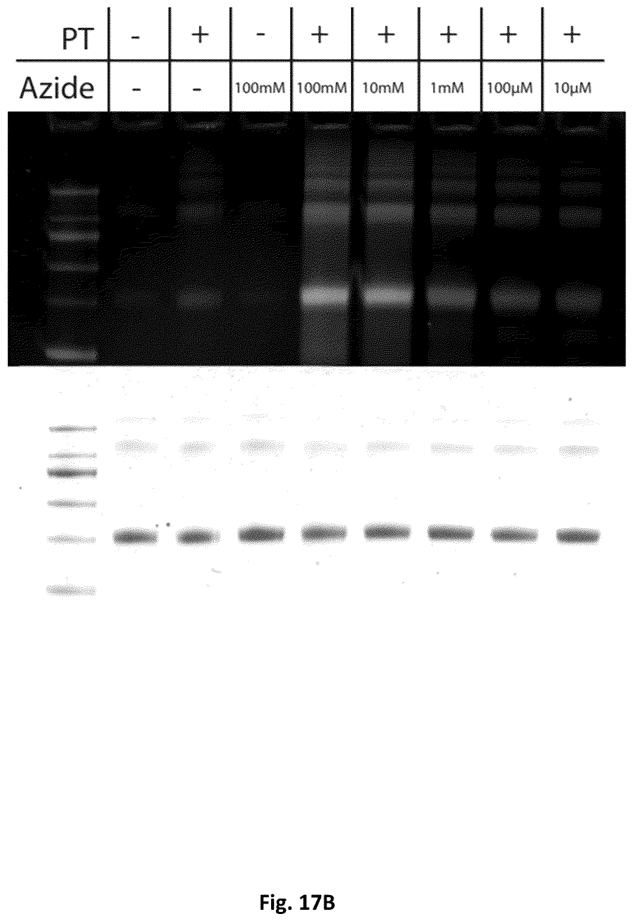

A non-limiting azidylation reaction with hydrogen peroxide as the oxidizing agent is made up with the following final concentrations and preferably executed in phosphate-buffered saline. The concentrations may be varied empirically; see, for example, the brief description of FIGS. 13A-17B.
BSA—about 5 to about 10 μM,
Sodium Azide—about 100 mM
H₂O₂—about 0.5 to about 2.0%.

Likewise, a non-limiting azidylation reaction using phenyliodosohydroxy tosylate (PT) as the oxidizing agent is made up with the following final concentrations and preferably run in phosphate-buffered saline. Conditions may vary; see, for example, the brief description of FIGS. 13A-17B:
BSA—about 5 to about 10 μM,
Sodium Azide—about 100 mM,
PT—≤about 200 μM.

After addition of components, reactions are gently vortex to homogeneity for 5 seconds, and let rest at room temperature for up to 30 seconds before putting on ice. Protein is then precipitated via methanol/chloroform precipitation, trichloroacetic acid precipitation, or desalted via Zeba spin tip C18 desalting. Click chemistry using both CuAAC and SPAAC were used here and done under aqueous conditions. For CuAAC reactions, the final concentrations of the components were as follows:
4 M Urea
50 mM Ammonium Bicarbonate
79 μM alkyne reactant (or equivalent volumes of H₂O or DMSO for control samples)
6.34 mM tris-hydroxypropyltriazolylmethylamine (THPTA)
1.26 mM cupric sulfate
19 mM sodium ascorbate

For SPAAC reactions, the final concentrations of the components were as follows:

19 μM dibenzocyclooctyne (DBCO) reagent (or equivalent volumes of H₂O or DMSO for control reactions).

For both click reactions, samples were reacted for 20-35 minutes at room temperature in the dark with mixing by end-over-end turning or gently vortexing.

Sample processing following the click reaction varied per experiment. For fluorescent alkyne and DBCO reagents, about 500 ng to about 5 μg of BSA was loaded onto a polyacrylamide gel and resolved via gel electrophoresis. The resulting gel was fluorescently imaged using a LiCor Odyssey system, and thereafter was stained with Coomassie dye and imaged with a generic computer scanner.

For alkyne/DBCO reagents containing biotin as the detectable reagent, about 500 ng to about 5 μg (equivalent amounts for both gels) of BSA was loaded onto two polyacrylamide gels and concurrently resolved via gel electrophoresis. One was Coomassie stained as above. Protein from the second was transferred to a Western blotting membrane, blocked for 1 hour with LiCor PBS-based bocking reagent, and incubated with 1 μL of LiCOR-brand streptavidin with rocking overnight in the cold and dark. Membrane was washed the following morning and imaged with the LiCOR Odyssey system.

From these same reactions, the remaining protein was precipitated with trichloroacetic acid precipitation and resolubilized into 8 M urea/50 mM ammonium bicarbonate and processed for mass spectrometry (MS) analysis as follows. Samples were diluted with 50 mM ammonium bicarbonate to 4 M urea, reduced with 2 mM dithiothreitol for 30 minutes at 42° C., alkylated with 5 mM iodoacetamide for 30 minutes at room temperature in the dark, and diluted to 1 M urea with 50 mM ammonium bicarbonate. A 1:1 mixture of the proteases Trypsin/LysC were added at a mass ratio 1:100 protease:protein, and samples were digested at 37° C. for 12 hours, then held at 2° C. until enrichment, below.

For enrichment, 50 μL of stock suspension of EZView Streptavidin resin (Millipore Sigma) was used per sample, and all centrifugation was at room temperature for 30 seconds and 8.2 K×g. In a 1.5 mL tube, resin was washed 2× with 1 mL 50 mM ammonium bicarbonate, then 2× with 1 mL 1 M urea/50 mM ammonium bicarbonate. To the equilibrated resin, total digests were added (˜400 μL). Peptides were bound to resin by end-over-end mixing in the cold and dark for between 2 and 4 hours. Unbound fractions were saved, and when specified, further processed as below (acidification, desalting, and solubilization), then analyzed by mass spectrometry. Resin with bound peptides was resuspended into 100 μL pure water and transferred to a thin-wall, 0.25 mL PCR tube. The tube was laid on its side and exposed to 365 nM light in the cold for 30 minutes to cleave the biotin tag and release modified peptides. Following photocleavage, peptides in water were removed from resin, and resin was discarded.

Unbound fractions and photocleaved modified peptides were acidified to 1.0% v/v formic acid with neat formic acid. Samples were cleaned up using either Agilent OMIX C18 tips (unbound fractions) or Pierce 10 μL C18 tips according to manufacturer protocols. Eluted, desalted peptides were dried in a vacuum centrifuge to completion, and resolubilized into MS-grade 0.1% formic acid for MS analysis. Unbound fractions were solubilized into 50 μL to 100 μL and photocleaved peptides were solubilized into 10 μL. 0.5 μL of unbound fractions were used for analysis, and 1-3 μL of photocleaved peptides were used. LC-MS systems used were a Thermo Scientific UltiMate 3000 RSLC nano liquid chromatographic system and a Thermo Scientific Orbitrap Fusion Lumos Tribrid Mass Spectrometer. All flow rates were 300 nL/min, mobile phase A was 0.1% formic acid, and mobile phase B was 80% acetonitrile/0.1% formic acid. Sample was loaded onto a 50 mm Thermo Fisher Easy Spray HPLC column loaded with 2 μm particle size and 75 μM diameter under 2% B conditions and peptides were eluted with a gradient from 5-37.5% B over 38 minutes. A spray voltage of 1900V was used for electrospray ionization. For mass spectral data acquisition, the parameters were as follows: MS1 data was collected in positive mode in the Orbitrap mass analyzer in profile mode with a resolving power of 120K, a scan range of 350-1600 m/z, and a normalized automatic gain control (“AGC”) Target of 250%. To select ions for MS2 analysis, monoisotopic peak selection was set to peptide mode and a charge state filter of +2-7 was used. Cycle time between MS1 spectra was set to 1 s. MS2 analysis also occurred in the Orbitrap mass analyzer at 30K resolution. When high-energy collisional dissociation (“HCD”) fragmentation was used, HCD collision energy % was set to 32, and mass range was set to normal and determined automatically per analyte. Quadrupole isolation was used with a window of 0.7 m/z and a normalized AGC target value of 300% was used. When electron-transfer dissociation (“ETD”) fragmentation was used, calibrated charge-dependent ETD parameters were automatically selected, and mass analysis, isolation, and AGC values were as described above for HCD. When supplemental HCD was used in conjunction with ETD (EThcD), a supplemental collision energy of 15% was used. All MS2 spectra were acquired as centroid data.

Raw data was analyzed using Thermo Scientific Proteome Discoverer software and the SEQUEST algorithm for database searching. See Jimmy K. Eng, Ashley L. McCormack, and John R. Yates, III (1994) “An Approach to Correlate Tandem Mass Spectral Data of Peptides with Amino Acid Sequences in a Protein Database,” J Am Soc Mass Spectrom 5 (11): 976-989. Data was searched against a database containing common and lab-specific contaminant proteins with BSA inserted with the following parameters for HCD MS2 data as described above:
Peptides were specified as tryptic with 2 missed cleavages allowed
MS1 mass tolerance was set to 10 ppm
MS2 mass tolerance was set to 0.1 Da
b and y fragment ions were considered
variable oxidation was specified on every residue expect cysteine
variable adduct of +96.04 (the result of azidylation, clicking to a photocleavable biotin tag, and cleaving off the biotin) was specified on every residue except cysteine,
carbamidomethylation was set as fixed on cysteine residues.

A maximum of four (4) modifications per peptide was allowed. For ETD and EThcD MS2 data as described above, all parameters were the same save for considering c and z fragment ions as well. For all data, a decoy database search strategy was employed and a false discovery rate of 0.01 was used to filter resultant data.

Arabidopsis Cytosol for the gel displayed was extracted by blending in general homogenization buffer with protease inhibitors added and spun at high speed to clarify membranes and particulate.

Results of this Example are shown in FIGS. 13A-17B and the brief description of the figures.

### Example 3

This Example demonstrates that lysozyme, a second standard protein alongside BSA can be azidylated using the method disclosed herein, and that the downstream clicking, enrichment, and MS method described in Examples 1 and 2 can similarly identify regions of modification.

Here, we contacted lysozyme with 1 mM, 10 mM, 100 mM or 500 mM azide, and compared the effects of with or without the oxidizing agent H₂O₂. The products were then contacted with biotinylated alkyne for click reaction.

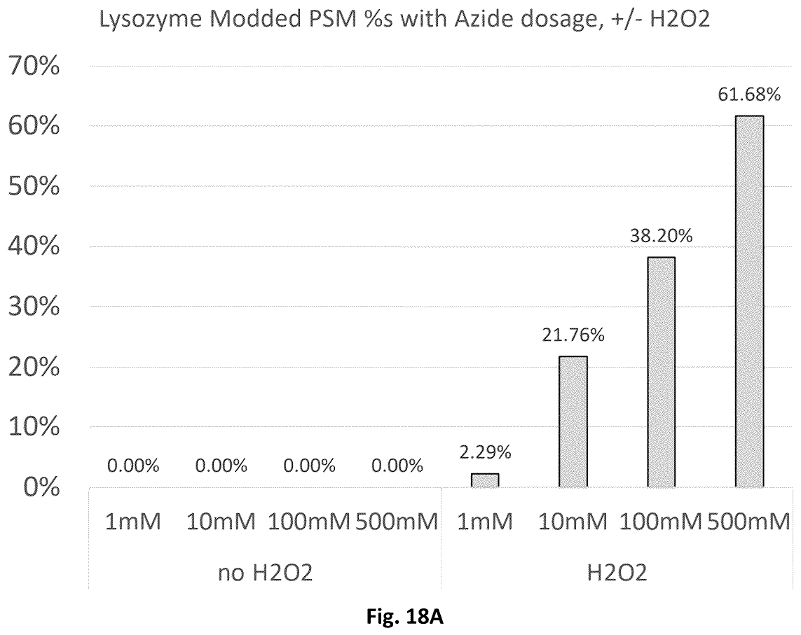

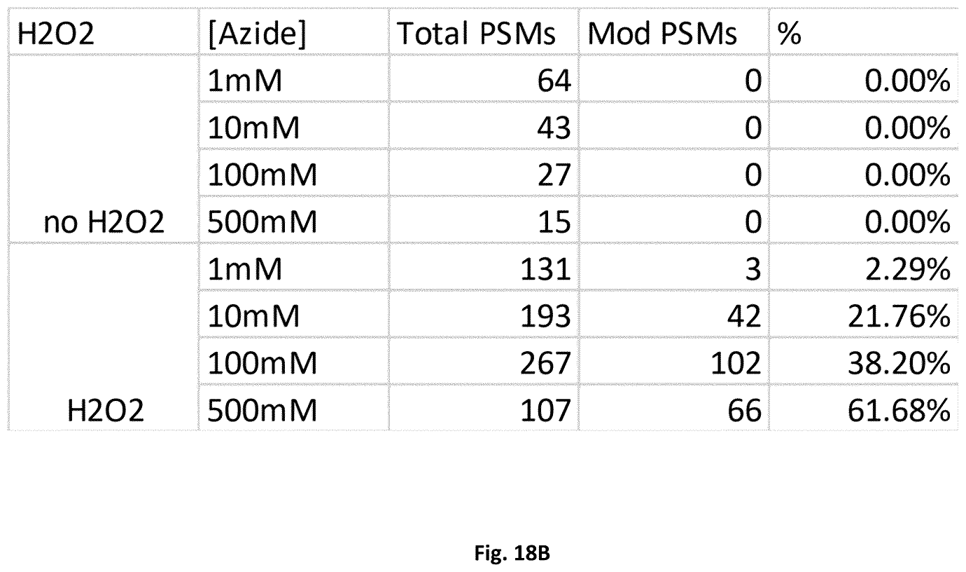

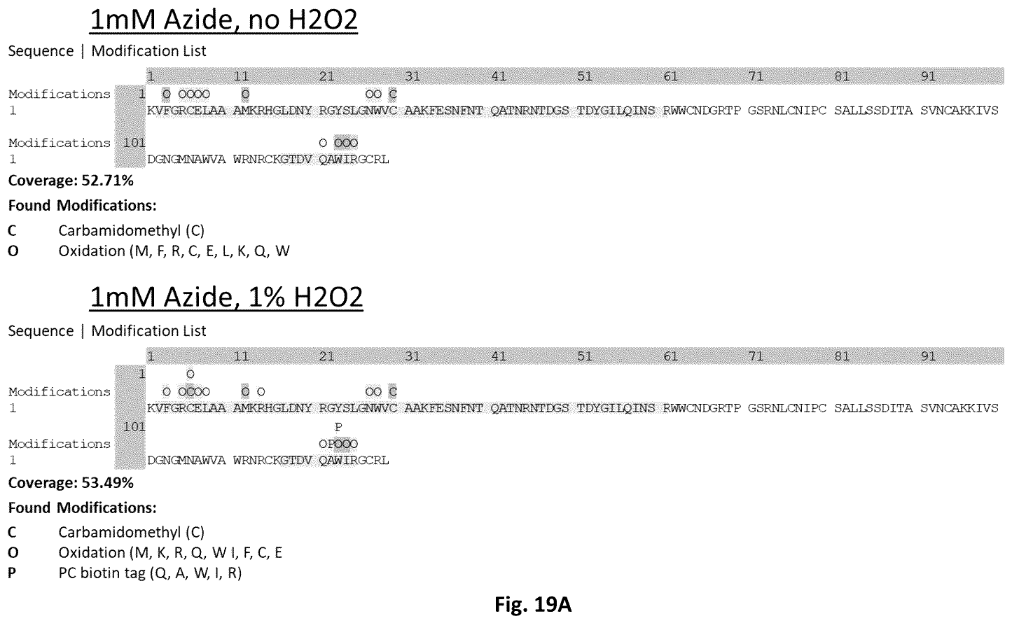

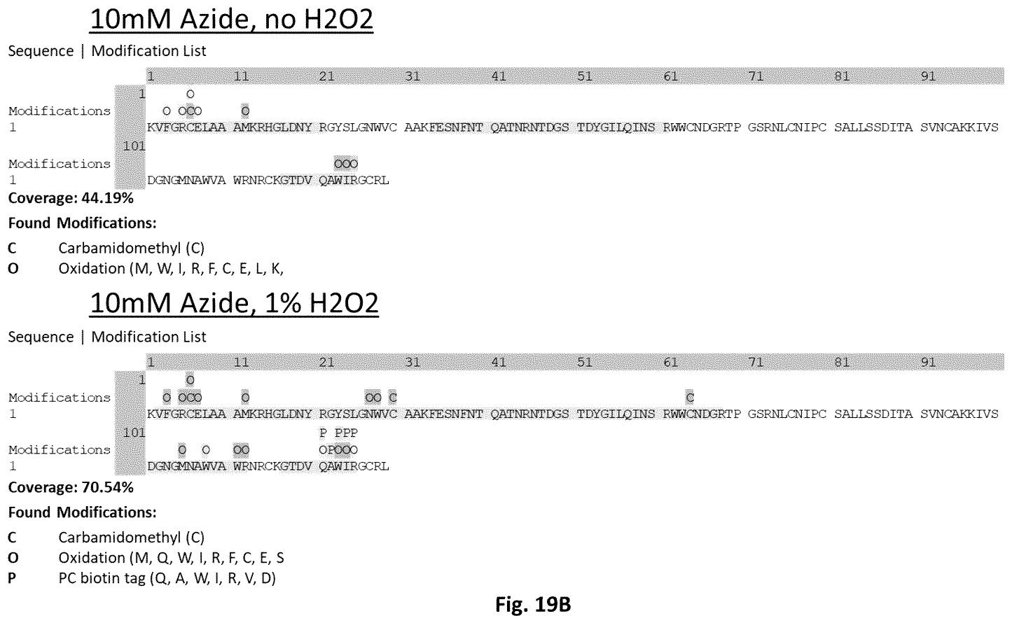

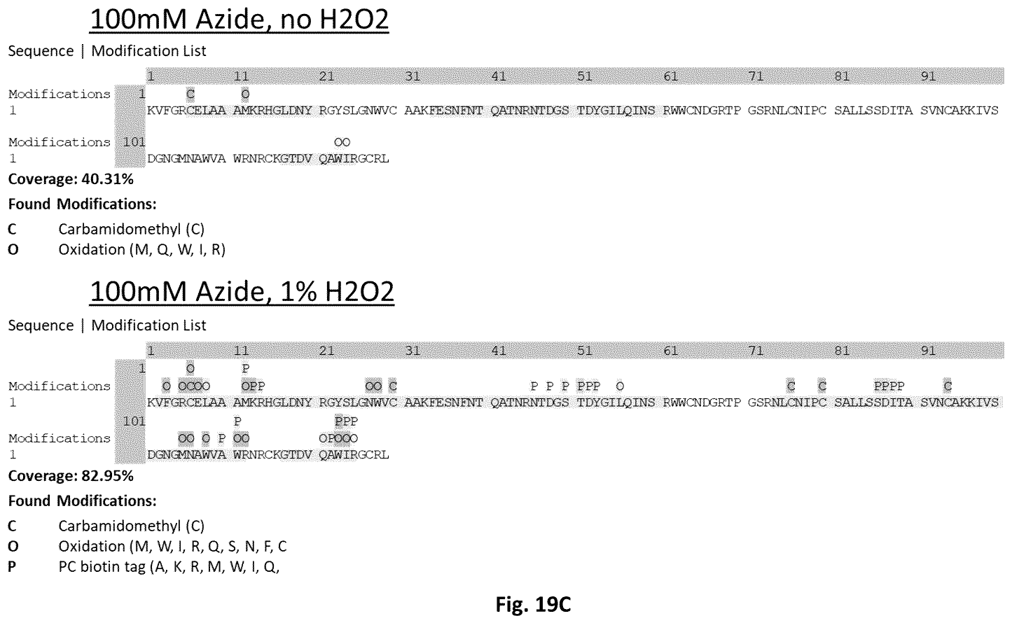

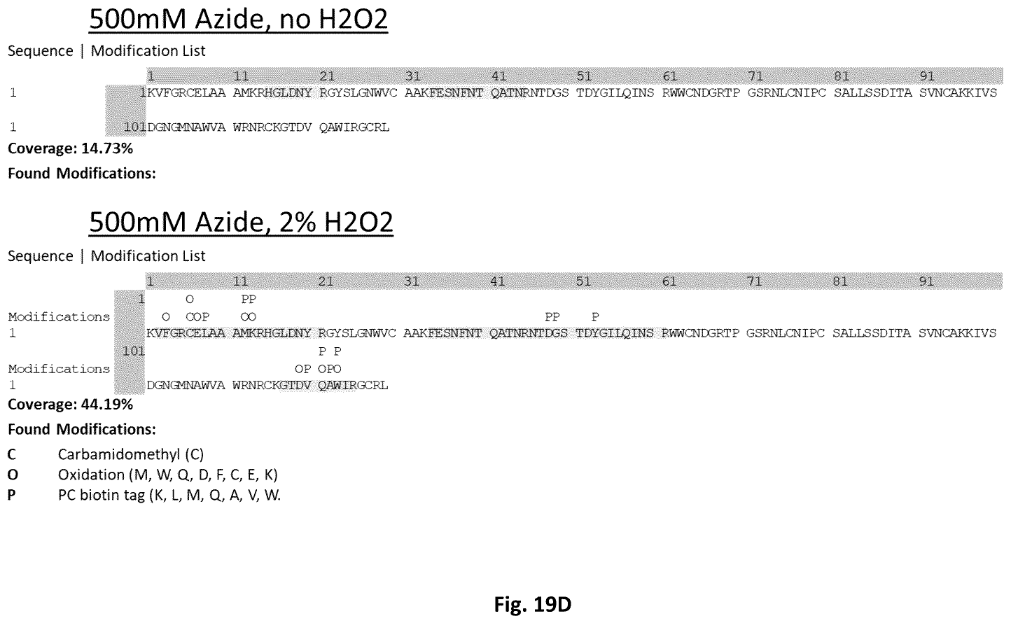

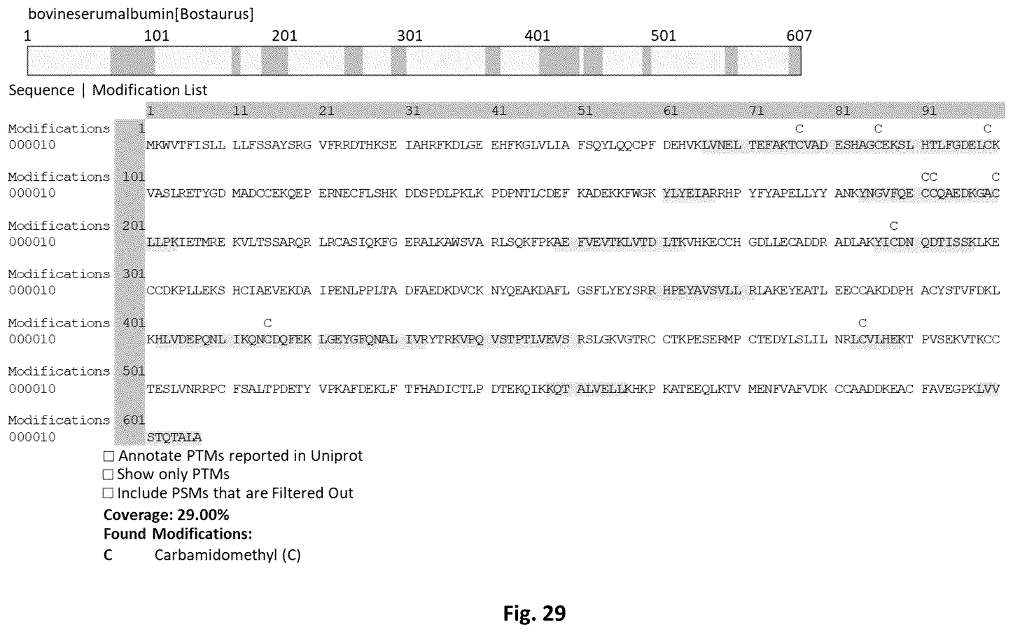

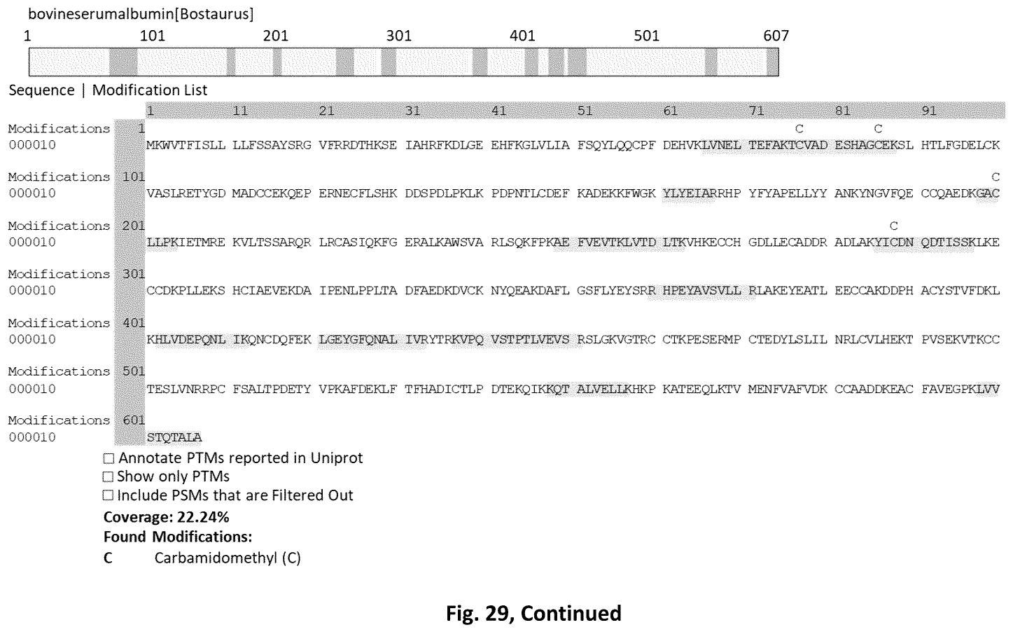

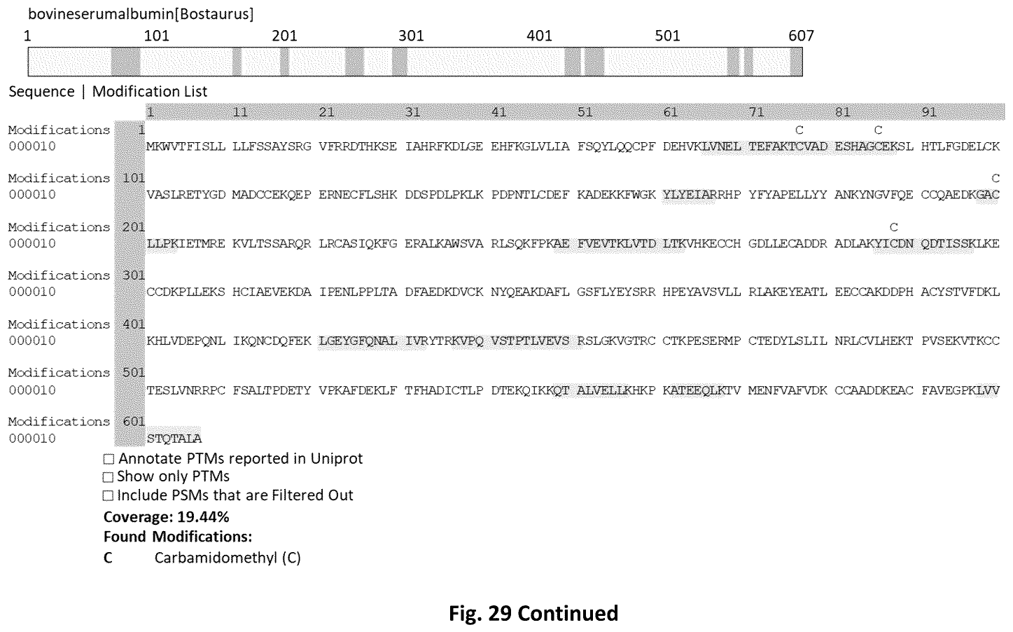

FIGS. 218A and 18B show that azidylation of lysozyme is H₂O₂-dependent and azide dose-dependent. Without the presence of H₂O₂, azidylation of lysozyme did not occur. With the presence of H₂O₂, azidylation all occurred, and the percentage of modified lysozyme increased with the increase of azide concentration. Note that we started to lose peptide IDs with 500 mM azide (FIG. 29B), probably due to cleavage events. The clicking, enrichment and MS analysis following azidylation shows the mass adduct of the PC biotin tag to the azidylated lysozyme (FIGS. 19A-19D).

### Example 4

This Example verifies the “one-pot” click method by reacting BSA with azide and biotinylated alkyne using the conditions of a normal CuAAC reaction. The azide concentrations of 10 μM, 100 μM, 1 mM, and 10 mM were examined. The reaction was run at room temperature in dark, either resting for 20 min after mixing or vortexing for 20 min. The method of digest, enrichment, and MS analysis following the “one-pot” click reaction is as described in Example 2.

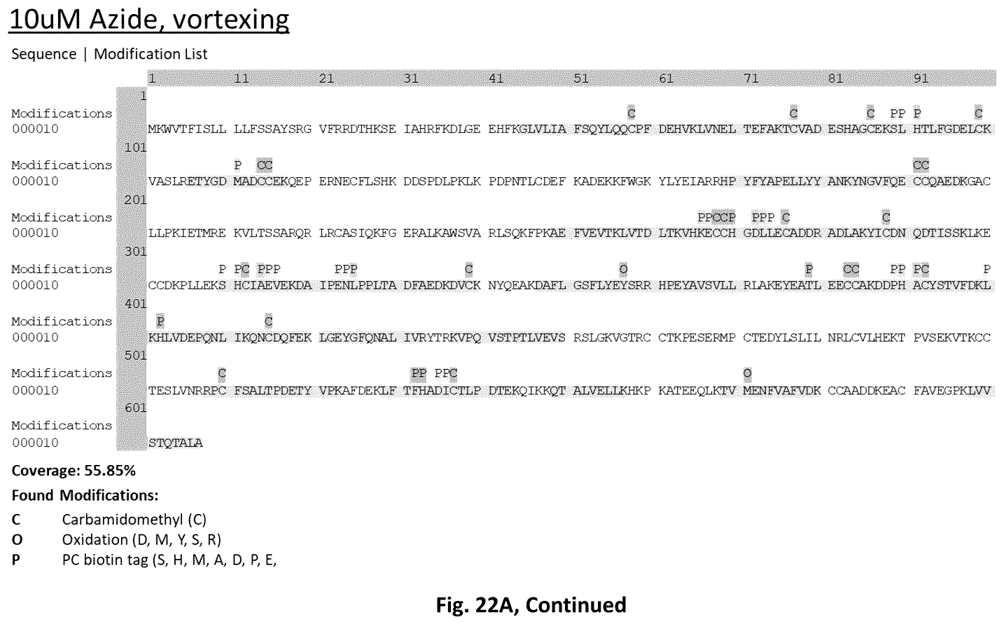

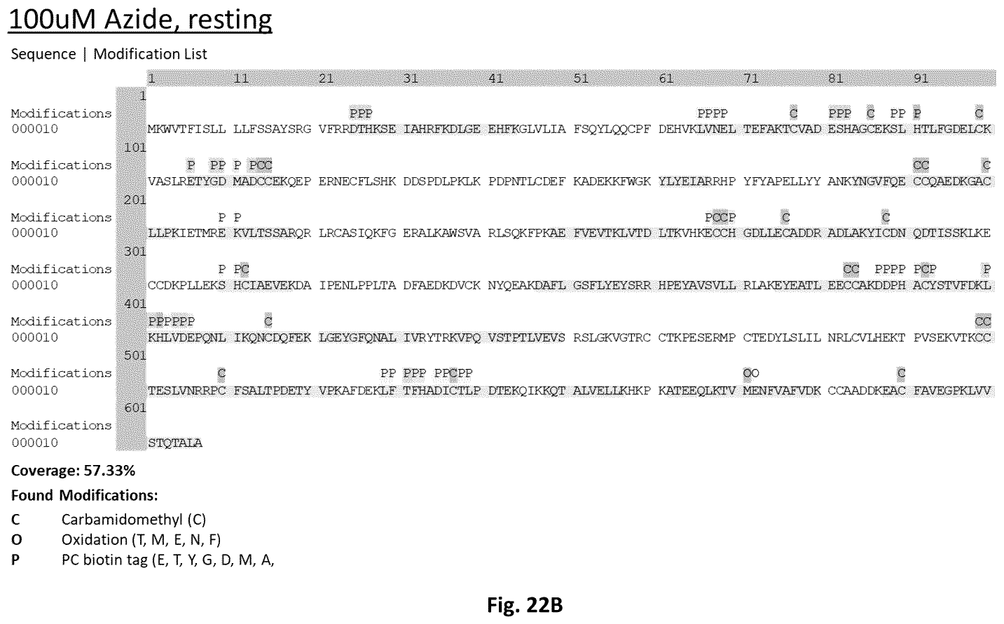

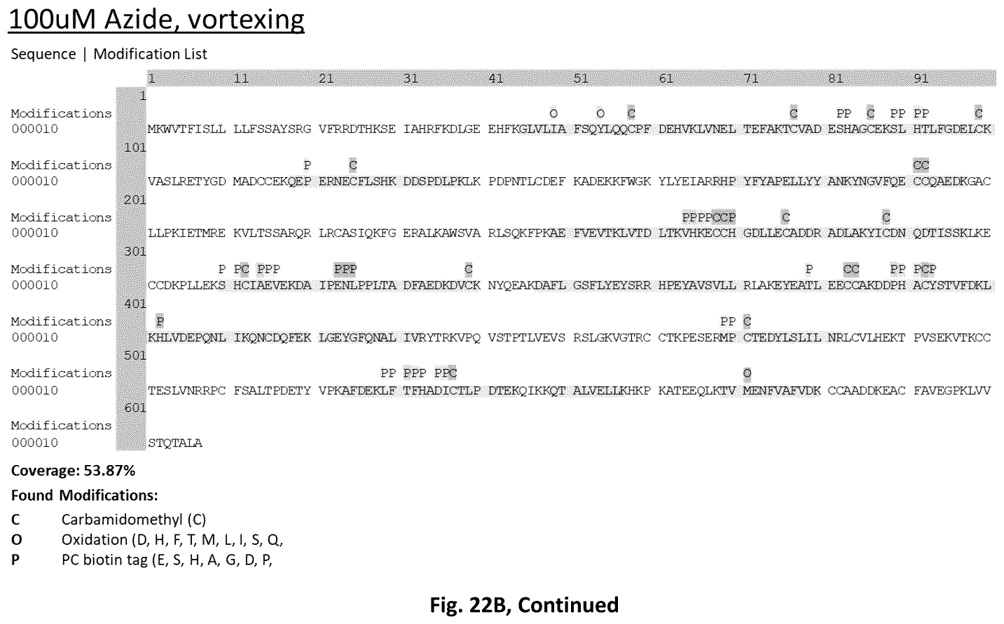

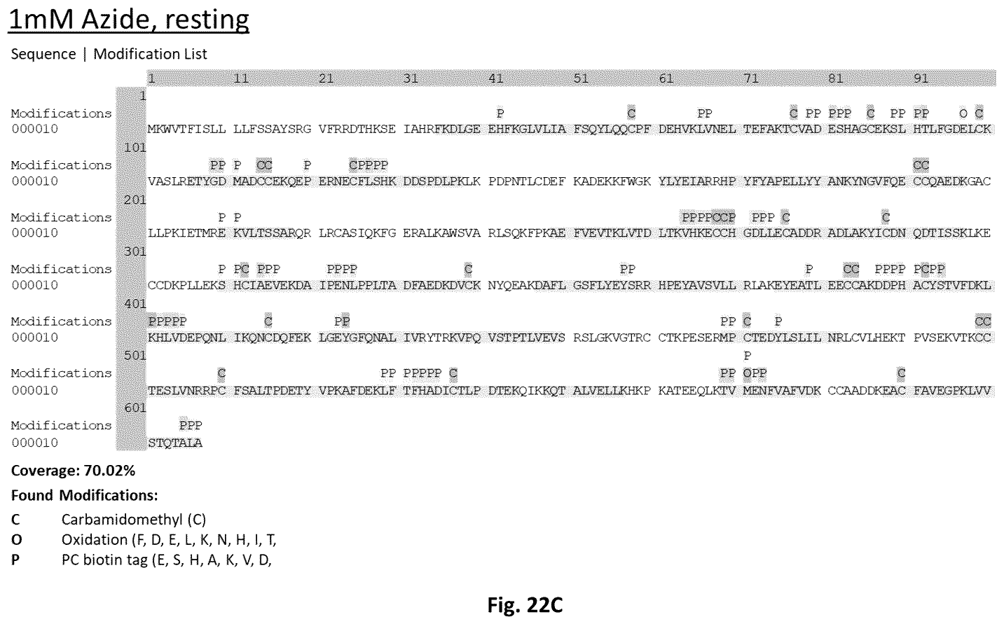

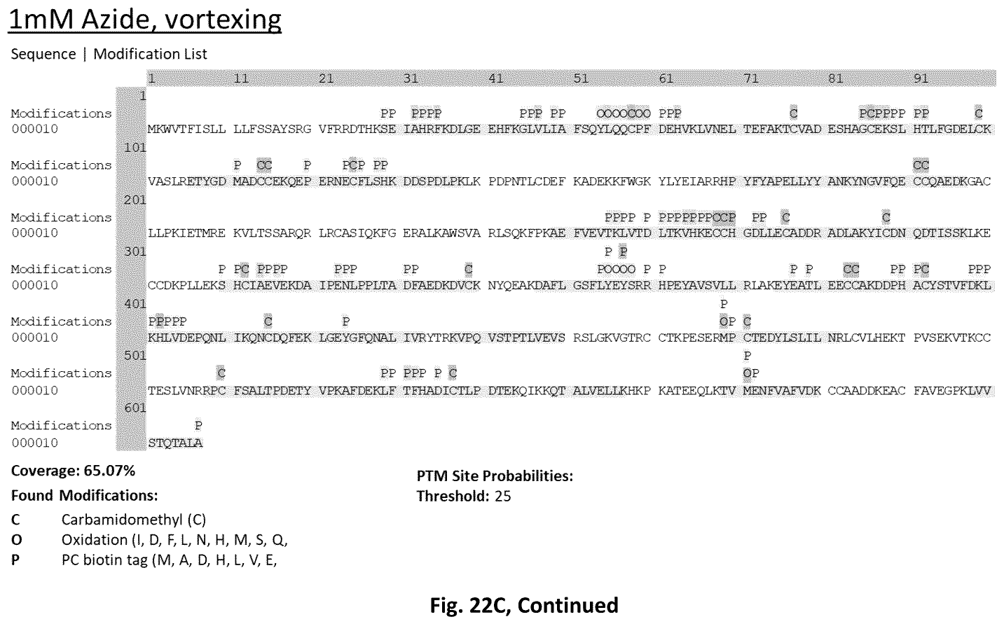

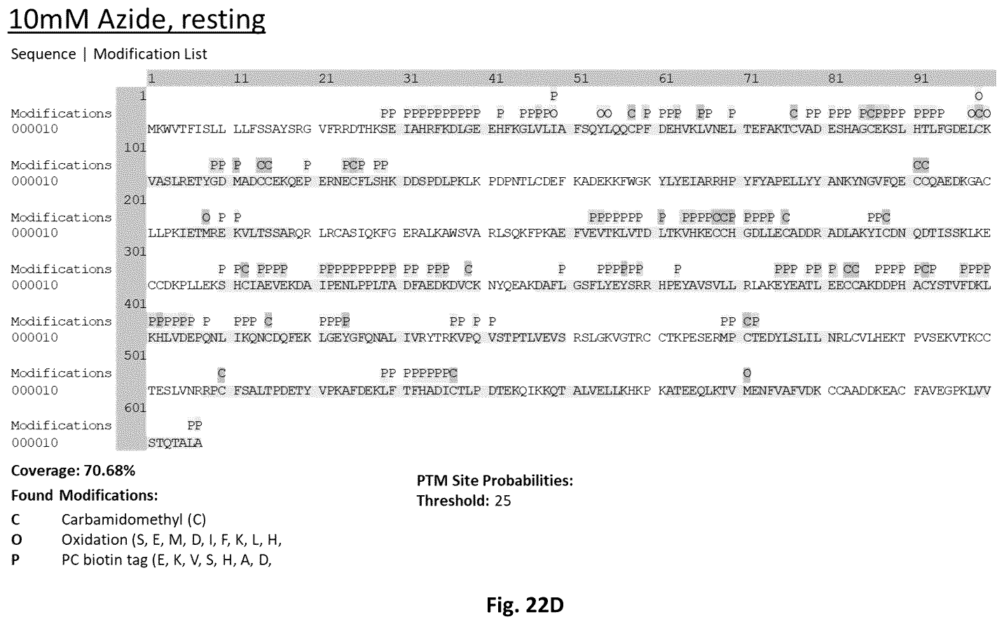

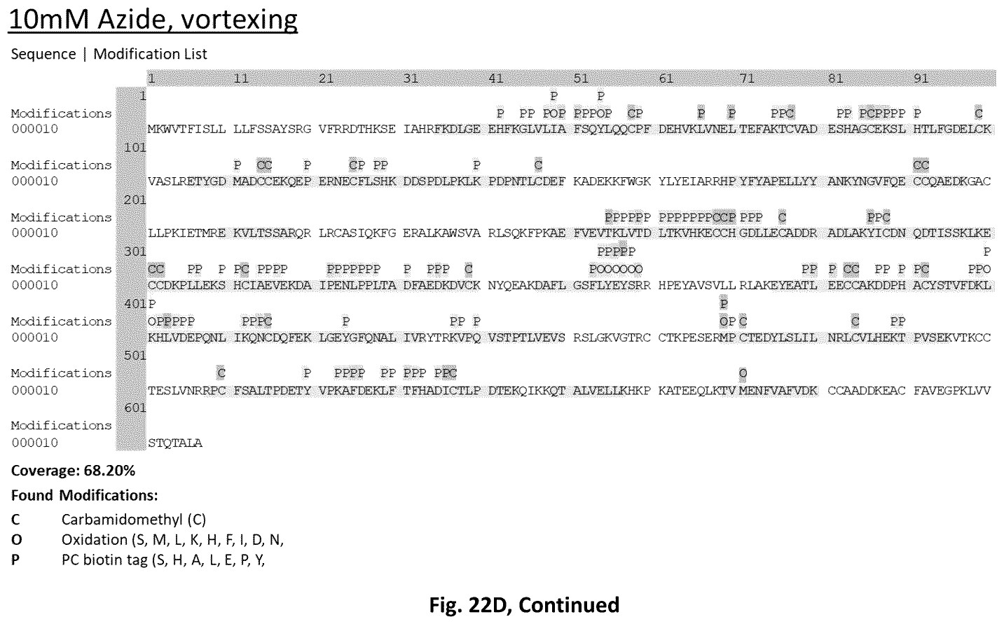

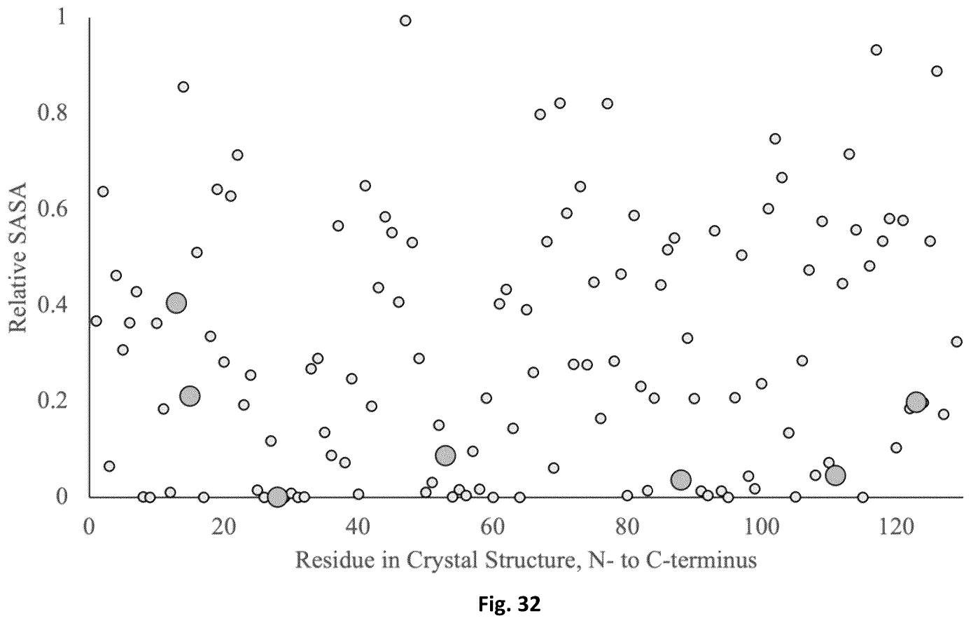

The modification of BSA in the “one-pot” click method shows dose-dependence with azide concentration (FIG. 32). Vortexing samples during the reaction increased modification of BSA compared with letting the mixture sitting, and the discrepancy is more evident at lower azide concentrations, such as 10 and 100 μM. The result is not surprising, as vortexing would increase the reaction efficiency and add some oxidation. No modification of BSA was observed for the reaction with 10 μM azide and without vortexing. The enrichment and MS analysis following the “one-pot” click reaction shows the mass adduct of the PC biotin tag except the reaction with 10 μM azide and without vortexing (FIGS. 22A-22D).

### Example 5

### Summary

The ability of a protein's amino acid chains to fold into a diverse array of naturally occurring catalytic structures combined with high throughput protein engineering technologies holds great promise for new innovations in synthetic organic chemistry (1). Critical to this work is a precise understanding of the dynamic motion of the amino acid side chains in an aqueous solution, rather than frozen after vitrification into a specific conformer during cryo-EM or in a crystal prior to analysis by X-ray diffraction. A plethora of studies on the folding and unfolding of proteins has indicated that the shielding of short hydrophobic patches from aqueous solution is an important driving force for these dynamic processes (2, 3). Although NMR can provide important clues on the dynamic motion, these studies are limited to the few proteins that are small enough and can be purified to large amounts for analysis with electromagnetic radiation. To study in-solution protein dynamics, mass spectrometry-based ‘footprinting’ using covalent amino acid modifying reagents such as hydroxyl (OH) radicals (HRF, for hydroxyl radical footprinting) has emerged as a field that can provide considerable insights without as great a limitation on size, purity and amount (4). There are many ways to perform HRF, including synchrotron-induced, pulsed laser-induced, or plasma-induced radical generation (4). Footprinting provides critical information on location of solvent accessible amino acid side chains, corroborates information derived from high resolution structures determined by traditional means (5), and with appropriately modified labeling reagents and conditions, can modify residues found in transmembrane domains (6, 32, 33).

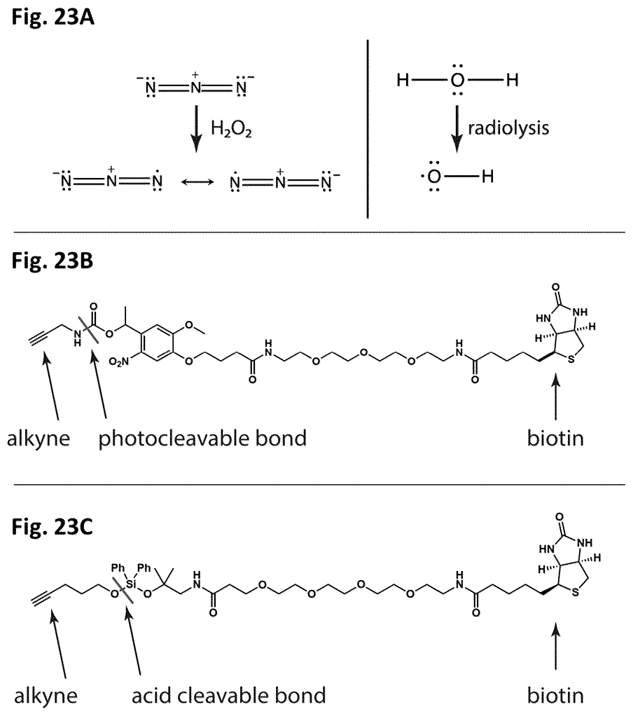

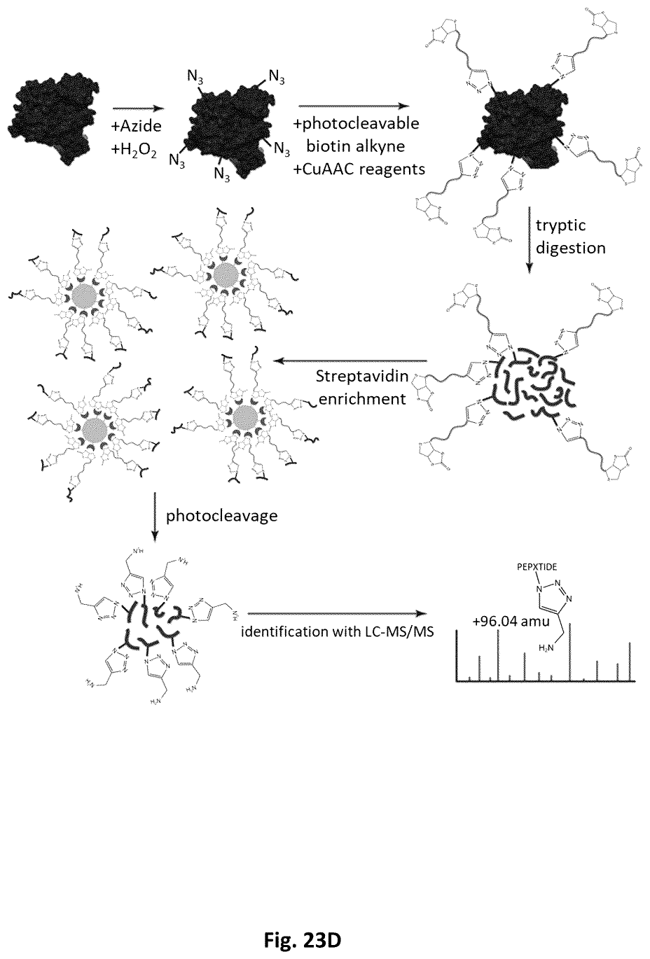

Within this context, we began to consider the azido radical for protein footprinting. Whereas the OH radical is a small, diatomic molecule that lacks delocalized electron density, the azido radical is triatomic and exhibits resonance that delocalizes the singlet electron across the three nitrogen atoms (FIG. 23A), thus providing the capability of short-range pi electron attraction with other aromatic molecules as well as with electron deficient centers, such as cations. Given these differences with the hydroxyl radical, we reasoned that developing a method to label protein in solution with azide radicals might complement existing protein footprinting methods and enable azide-based click derivatization for synthetic protein chemistry (34). The majority of azide addition chemistry reported in the literature, whether the mechanism involves the azide anion or the neutral azido radical, occurs in organic solvents or in organic/aqueous solvent mixtures and temperatures or pH that are deleterious to proteins maintaining their native, catalytically active three dimensional folded state (7, 35). Because of this, the known body of literature on azidylation chemistry is inapplicable to studying properly folded native protein structure. That said, a few reports indicate that azido radicals can covalently modify free amino acids (8-12), though the majority of data obtained to date rather suggests that azido radicals create transient sidechain radicals which create structures such as dityrosine bridges that lack the azide adduct (13). The suggestion that azido radicals can covalently attack aromatic amino acids or ringed olefins (9, 10) led us to ask whether azido radicals could directly modify amino acid sidechains in an intact 3D structured protein context, an observation that has not yet been reported.

There are currently three methods to introduce an azide group into a protein, but none are truly satisfactory. First, one can supplement mRNA translation with azide-containing residues in permissible cellular backgrounds (14, 15), second, azide containing reagents that perform site-specific chemical derivatization, such as maleimide chemistry, N-hydroxysuccinimide (NHS) ester chemistry, or N-terminal derivatization using a pyridine compound (16) can be utilized, or third, derivatization of primary amines to azides using diazo transfer (17, 18) can be performed. Of these, diazo transfer without free radicals is most comparable to the radical based method described herein, but is limited in that at physiological pH, few amines undergo the conversion, the process takes many hours and requires specialized reagents not readily available (18). We reasoned that a method that relies upon azido free radical attack and requires only simple, inexpensive, and safe reagents would have widespread utility for protein labelling as a prelude for its additional use in understanding protein structure and function.

Herein, we report multiple novel findings. First, simply adding sodium azide and H₂O₂ to protein in physiological buffer and at neutral pH causes direct azido radical attack and covalent azidylation on multiple residues, which can then be derivatized using the click reaction copper-catalyzed azide-alkyne cycloaddition (CuAAC) to add an enrichable handle to covalently bound azide groups (34, 36). This is demonstrated using bovine serum albumin (BSA), lysozyme, and a complex soluble protein mixture from an Arabidopsis cell lysate. Second, although we have not extensively examined this, noncovalent azide:protein binding may be a more universal phenomenon than previously thought from studies focused on metabolically required enzymes that it targets, such as cytochrome c oxidase and the F1-ATPase (37). We find that both BSA and lysozyme can bind and supply azide to the copper-catalyzed click reaction and enable concurrent azidylation and azide-alkyne cycloaddition. Most important, our cumulative data support a model in which hydrophobicity, rather than solvent accessibility, is the main driving factor in azide:protein binding, an observation that is directly opposite to that seen with hydroxyl radicals and other widely used covalent modifying reagents. Covalent azide modification occurs mainly, but not exclusively, on residues that are buried in atomic resolution 3D structures, within catalytic active sites, in ligand-binding regions, and remarkably close to co-crystallized azide binding sites. Importantly, we have discovered that covalent azidylation is totally dependent upon 3D structure and can be outcompeted with known hydrophobic ligands such as fatty acids and 8-analinonapthelene-1-sulfonic acid (ANS), a fluorescent reporter widely used to study renaturation and surface hydrophobicity of soluble proteins (38). Overall, we found that the azide anion can be oxidatively radicalized to create a covalent modifying reagent, and we present a model in which azide binds both covalently and noncovalently to hydrophobic amino acid patches within proteins' three-dimensional structures. This modification can be captured and localized using mass spectrometry, and altogether comprises the first method for empirically assaying three-dimensional protein hydrophobic microenvironments or ‘patches’, with a covalent molecular probe.

### Results

### Method Development and Rationalization:

Classically, azido radicals that interact with free amino acids are generated by irradiating water to create hydroxyl radicals, which in turn react with azide to produce azido radicals (10, 11). Given this, we asked whether more accessible methods of oxidation could facilitate azido radical generation in a buffer system amenable to native protein. H₂O₂ was selected as the oxidant due to the relative ease of use, availability, and reported oxidizing capability in a protein context (19, 20). A publication that reports addition of azido radicals to free tryptophan suggests that the adduct reaction is inefficient and produces a product with relatively low abundance (10). Thus, we developed an assay to detect and enrich for the modified compound using the click chemistry reaction copper-catalyzed azide-alkyne cycloaddition (CuAAC) (39). Using a biotinylated and either acid- or photocleavable alkyne allows capture with streptavidin followed by release using acid or light (FIGS. 23B and 23C). These alkynes were chosen because the resulting triazole-linked biotin is detectable with both streptavidin blotting and tandem mass spectrometry (MS/MS). In our procedure, a protein is first oxidatively azidylated by treatment with sodium azide and H₂O₂ at ambient temperature and physiological pH (7.0). Next, the azidylated is derivatized to a triazole linkage using CuAAC and an alkyne, as shown in FIGS. 23B and 23C. To monitor azidylation, one can utilize either routine SDS-PAGE Western blotting procedures or bottom-up mass spectrometry. Briefly, the protein can be separated via SDS-PAGE and blotted with streptavidin to detect clicked-on biotin and, for mass spectrometric-based analysis, protein can be proteolytically digested and the resulting modified peptides enriched with streptavidin and eluted via acid or photocleavage to produce peptides with triazole-containing mass adducts of +125.06 amu or +96.04 amu, respectively, (FIG. 23D) whose chemical identity and location can be definitively determined via high resolution tandem mass spectrometry.

### Oxidative Azidylation Modifies Lysozyme:

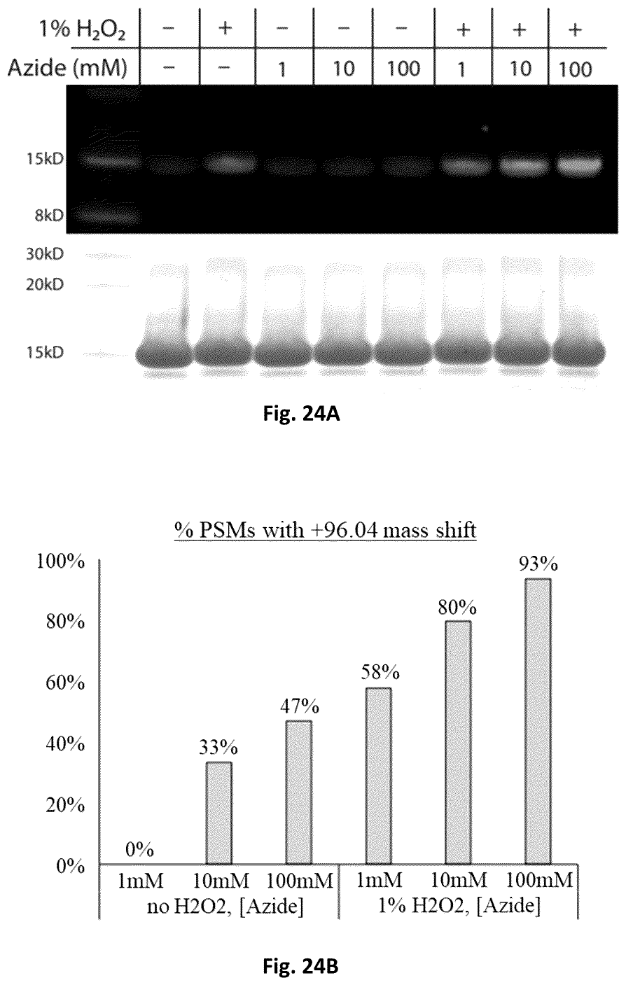

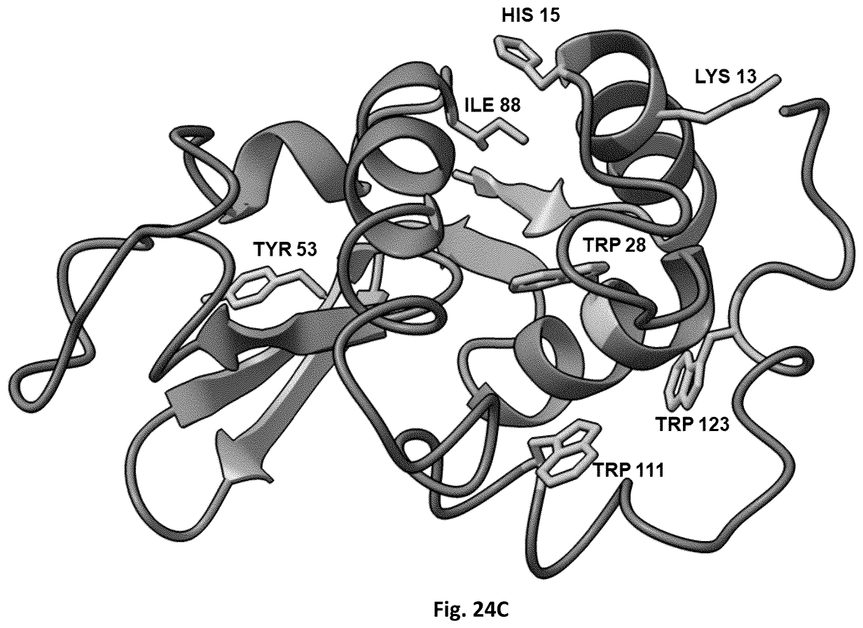

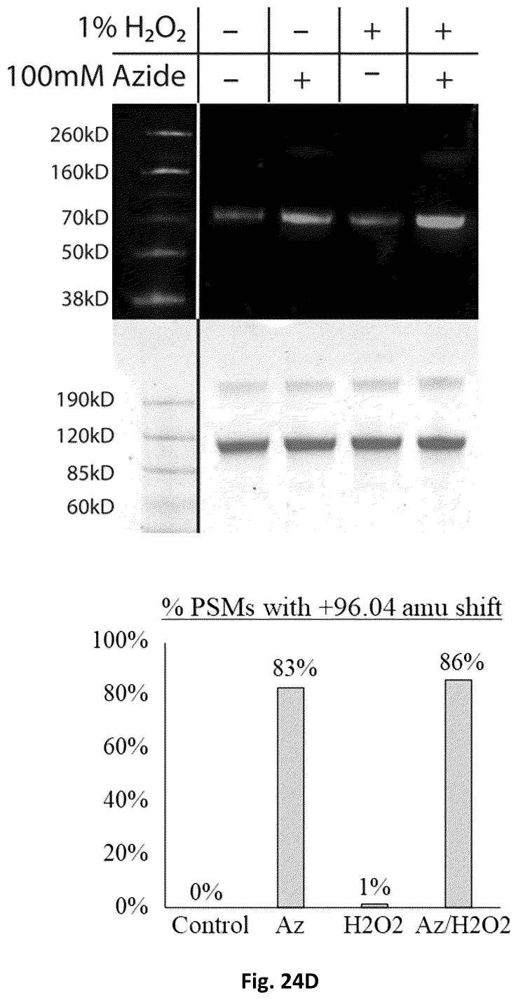

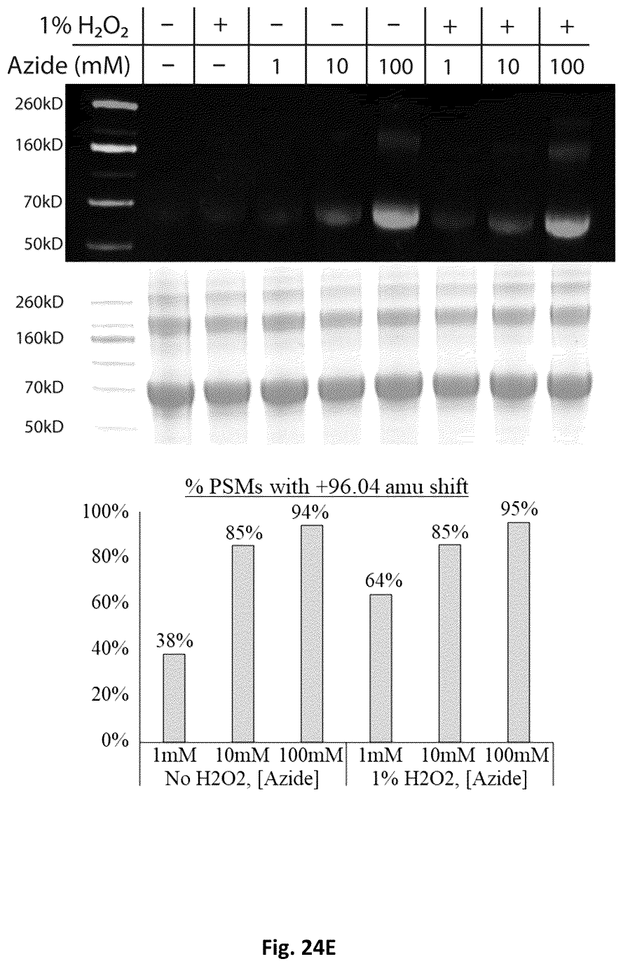

As shown in FIG. 24A, by adding 1% H₂O₂ and various concentrations of azide to lysozyme, clickable azidylation of lysozyme was detected using both blotting and MS. H₂O₂ addition alone led to a minor increase in blotting, though adding azide to the reaction significantly increased the signal, and, as shown below, H₂O₂ alone does not lead to azidylation as observed with MS. For further insight into the mechanisms underlying the gel-based observations, high resolution tandem mass spectrometry was used to confirm the modification mass adduct, to localize azidylation sites, and to measure the degree of azidylation (FIG. 24B). High energy collisional dissociation (HCD) fragmentation of peptides containing the azide modification revealed that under these conditions the triazole adduct is partially labile but could be retained by using electron transfer dissociation with supplemental collisional activation (EThcD), evidenced by both a richer series of fragment ions and presence of unfragmented precursor. See Tables 9-15.

TABLE 9
A representative ion table of BSA peptide DAFLGSFLYEYSR
(SEQ. ID. NO: 31), containing the +96.04 modification,
“PC biotin,” on Tyrosine 9.
Ion Series | Neutral Losses | Precursor Ions | Internal Fragments

| #1 | b⁺ | b²⁺ | Seq. | y⁺ | y²⁺ | #2 |
|---|---|---|---|---|---|---|
| 1 | 114.09134 | 57.54931 | L | | | 13 |
| 2 | 242.18630 | 121.59679 | K | 1529.85944 | 765.43336 | 12 |
| 3 | 475.29357 | 238.15042 | H-PC | 1401.76448 | 701.38588 | 11 |
| | | | bioti . . . | | | |
| 4 | 588.37763 | 294.69245 | L | 1168.65721 | 584.83225 | 10 |
| 5 | 687.44604 | 344.22666 | V | 1055.57315 | 528.29021 | 9 |
| 6 | 802.47299 | 401.74013 | D | 956.50474 | 478.75601 | 8 |
| 7 | 931.51558 | 466.26143 | E | 841.47779 | 421.24254 | 7 |
| 8 | 1028.56834 | 514.78781 | P | 712.43520 | 356.72124 | 6 |
| 9 | 1156.62692 | 578.81710 | Q | 615.38244 | 308.19486 | 5 |
| 10 | 1270.66985 | 635.83856 | N | 487.32386 | 244.16557 | 4 |
| 11 | 1383.75391 | 692.38059 | L | 373.28093 | 187.14410 | 3 |
| 12 | 1496.83798 | 748.92263 | I | 260.19687 | 130.60207 | 2 |
| 13 | | | K | 147.11280 | 74.06004 | 1 |

TABLE 10
Fragment ion table of peptide LKHLVDEPQNLIK (SEQ. ID. NO: 16), obtained
from EThcD fragmentation (ETD with supplemental HCD activation).
Ion Series | Neutral Losses | Precursor Ions | Internal Fragments

| #1 | c⁺ | c⁺H⁺ | c⁺H²⁺ | c²⁺ | c⁻H⁺ | c⁻H²⁺ | Seq. |
|---|---|---|---|---|---|---|---|
| 1 | 131.11789 | 132.12571 | 66.56650 | 66.06258 | 130.11006 | 65.55867 | L |
| 2 | 259.21285 | 260.22068 | 130.61398 | 130.11006 | 258.20503 | 129.60615 | K |
| 3 | 492.32011 | 493.32794 | 247.16761 | 246.66370 | 491.31229 | 246.15978 | H-PC biot . . . |
| 4 | 605.40418 | 606.41200 | 303.70964 | 303.20573 | 604.39635 | 302.70181 | L |
| 5 | 704.47259 | 705.48042 | 353.24385 | 352.73993 | 703.46477 | 352.23602 | V |
| 6 | 819.49954 | 820.50736 | 410.75732 | 410.25341 | 818.49171 | 409.74949 | D |
| 7 | 948.54213 | 949.54995 | 475.27861 | 474.77470 | 947.53430 | 474.27079 | E |
| 8 | 1045.59489 | 1046.60272 | 523.80500 | 523.30108 | 1044.58707 | 522.79717 | P |
| 9 | 1173.65347 | 1174.66129 | 587.83429 | 587.33037 | 1172.64564 | 586.82646 | Q |
| 10 | 1287.69640 | 1288.70422 | 644.85575 | 644.35184 | 1286.68857 | 643.84792 | N |
| 11 | 1400.78046 | 1401.78829 | 701.30778 | 700.89387 | 1399.77264 | 700.38996 | L |
| 12 | 1513.86452 | 1514.87235 | 757.93981 | 757.43590 | 1512.85670 | 756.93199 | I |
| 13 | | | | | | | K |

| | | | | | | | | #2 |
|---|---|---|---|---|---|---|---|---|
| y⁺ | y²⁺ | z⁺ | z⁺H⁺ | z⁺H²⁺ | z²⁺ | z⁻H⁺ | z⁻H²⁺ | 13 |
| 1529.85944 | 756.43336 | 1514.84854 | 1515.85637 | 758.43182 | 757.92791 | 1513.84072 | 757.42400 | 12 |
| 1401.76448 | 701.38588 | 1386.75358 | 1387.76140 | 694.38434 | 693.88043 | 1385.74575 | 693.37651 | 11 |
| 1168.65721 | 584.83225 | 1153.64632 | 1154.65414 | 577.83071 | 577.32680 | 1152.63849 | 576.82288 | 10 |
| 1055.57315 | 528.29021 | 1040.56225 | 1041.57008 | 521.28868 | 520.78476 | 1039.55443 | 520.28085 | 9 |
| 956.50474 | 478.75601 | 941.49384 | 942.50166 | 471.75447 | 471.25056 | 940.48601 | 470.74664 | 8 |
| 841.47779 | 421.24254 | 826.46689 | 827.47472 | 414.24100 | 413.73709 | 825.45907 | 413.23317 | 7 |
| 712.43520 | 356.72124 | 697.42430 | 698.43213 | 349.71970 | 349.21579 | 696.41648 | 348.71188 | 6 |
| 615.38244 | 308.19486 | 600.37154 | 601.37936 | 301.19332 | 300.68941 | 599.36371 | 300.18549 | 5 |
| 487.32386 | 244.16557 | 472.31296 | 473.32079 | 237.16403 | 236.66012 | 471.30514 | 236.15621 | 4 |
| 373.28093 | 187.14410 | 358.27003 | 359.27786 | 180.14257 | 179.63865 | 357.26221 | 179.13474 | 3 |
| 260.19687 | 130.60207 | 245.18597 | 246.19379 | 123.60054 | 123.09662 | 244.17814 | 122.59271 | 2 |
| 147.11280 | 74.06004 | 132.10191 | 133.10973 | 67.05850 | 66.55459 | 131.09408 | 66.05068 | 1 |

TABLE 11
Fragment ion table and spectrum of peptide LFTFHADICTLPDTEK
(SEQ. ID. NO: 17), obtained from HCD fragmentation.
Ion Series | Neutral Losses | Precursor Ions | Internal Fragments

| #1 | b⁺ | b²⁺ | Seq. | y⁺ | y²⁺ | #2 |
|---|---|---|---|---|---|---|
| 1 | 114.09134 | 57.54931 | L | | | 16 |
| 2 | 261.15975 | 131.08352 | F | 1890.88506 | 945.94617 | 15 |
| 3 | 362.20743 | 181.60735 | T | 1743.81665 | 872.41196 | 14 |
| 4 | 605.32420 | 303.16574 | F-PC biotin . . . | 1642.76897 | 821.88812 | 13 |
| 5 | 742.38311 | 371.69519 | H | 1399.65221 | 700.32974 | 12 |
| 6 | 813.42022 | 407.21375 | A | 1262.59329 | 631.80029 | 11 |
| 7 | 928.44717 | 464.72722 | D | 1191.55618 | 596.28173 | 10 |
| 8 | 1041.53123 | 521.26925 | I | 1076.52924 | 538.76826 | 9 |
| 9 | 1201.56188 | 601.28458 | C-Carbami . . . | 963.44517 | 482.22622 | 8 |
| 10 | 1302.60956 | 651.80842 | T | 803.41452 | 402.21090 | 7 |
| 11 | 1415.69362 | 708.35045 | L | 702.36685 | 351.68706 | 6 |
| 12 | 1512.74638 | 756.87683 | P | 589.28278 | 295.14503 | 5 |
| 13 | 1627.77333 | 814.39030 | D | 492.23002 | 246.61685 | 4 |
| 14 | 1728.82101 | 864.91414 | T | 377.20308 | 189.10518 | 3 |
| 15 | 1857.86360 | 929.43544 | E | 276.15540 | 138.58134 | 2 |
| 16 | | | K | 147.11280 | 74.06004 | 1 |

TABLE 12
Fragment ion table and spectrum of peptide LFTFHADICTLPDTEK (SEQ. ID. NO: 17),
obtained from EThcD fragmentation (ETD with supplemental HCD activation).
Ion Series | Neutral Losses | Precursor Ions | Internal Fragments

| #1 | c⁺ | c⁺H⁺ | c⁺H²⁺ | c²⁺ | c⁻H⁺ | c⁻H²⁺ | Seq. |
|---|---|---|---|---|---|---|---|
| 1 | 131.11789 | 132.12571 | 66.56650 | 66.06258 | 130.11006 | 65.55867 | L |
| 2 | 278.18630 | 279.19413 | 140.10070 | 139.59679 | 277.17848 | 139.09288 | F |
| 3 | 379.23398 | 380.24181 | 190.62454 | 190.12063 | 378.22616 | 189.61672 | T |
| 4 | 526.30240 | 527.31022 | 264.15875 | 263.65484 | 525.29457 | 263.15092 | F |
| 5 | 759.40966 | 760.41748 | 380.71238 | 380.20847 | 758.40183 | 379.70455 | H-PC bioti . . . |
| 6 | 830.44677 | 831.45460 | 416.23094 | 415.72702 | 829.43895 | 415.22311 | A |
| 7 | 945.47371 | 946.48154 | 473.74441 | 473.24050 | 944.46589 | 472.73658 | D |
| 8 | 1058.55778 | 1059.56560 | 530.28644 | 529.78253 | 1057.54995 | 529.27861 | I |
| 9 | 1218.58843 | 1219.59625 | 610.30176 | 609.79785 | 1217.58060 | 609.29394 | C-Carbami . . . |
| 10 | 1319.63611 | 1320.64393 | 660.82560 | 660.32169 | 1318.62828 | 659.81778 | T |
| 11 | 1432.72017 | 1433.72799 | 717.36764 | 716.86372 | 1431.71234 | 716.35981 | L |
| 12 | 1529.77293 | 1530.78076 | 765.89402 | 765.39010 | 1528.76511 | 764.88619 | P |
| 13 | 1644.79988 | 1645.80770 | 823.40749 | 822.90358 | 1643.79205 | 822.39966 | D |
| 14 | 1745.84755 | 1746.85538 | 873.93133 | 873.42742 | 1744.83973 | 872.92350 | T |
| 15 | 1874.89015 | 1875.89797 | 938.45262 | 937.94871 | 1873.88232 | 937.44480 | E |
| 16 | | | | | | | K |

| | | | | | | | | #2 |
|---|---|---|---|---|---|---|---|---|
| y⁺ | y²⁺ | z⁺ | z⁺H⁺ | z⁺H²⁺ | z²⁺ | z⁻H⁺ | z⁻H²⁺ | 16 |
| 1890.88506 | 945.94617 | 1875.87416 | 1876.88199 | 938.94463 | 938.44072 | 1874.86634 | 937.93681 | 15 |
| 1743.81665 | 872.41196 | 1728.80575 | 1729.81357 | 865.41043 | 864.90651 | 1727.79792 | 864.40260 | 14 |
| 1642.76897 | 821.88812 | 1627.75807 | 1628.76590 | 814.88659 | 814.38267 | 1626.75025 | 813.87876 | 13 |
| 1495.70056 | 748.35392 | 1480.68966 | 1481.69748 | 741.35238 | 740.84847 | 1479.68183 | 740.34455 | 12 |
| 1262.59329 | 631.80029 | 1247.58239 | 1248.59022 | 624.79875 | 624.29484 | 1246.57457 | 623.79092 | 11 |
| 1191.55618 | 596.28173 | 1176.54528 | 1177.55311 | 589.28019 | 588.77628 | 1175.53746 | 588.27237 | 10 |
| 1076.52924 | 538.76826 | 1061.51834 | 1062.52616 | 531.76672 | 531.26281 | 1060.51051 | 530.75889 | 9 |
| 963.44517 | 482.22622 | 948.43427 | 949.44210 | 475.22469 | 474.72078 | 947.42645 | 474.21686 | 8 |
| 803.41452 | 402.21090 | 788.40363 | 789.41145 | 395.20936 | 394.70545 | 787.39580 | 394.20154 | 7 |
| 702.36685 | 351.68706 | 687.35595 | 688.36377 | 344.68552 | 344.18161 | 686.34812 | 343.67770 | 6 |
| 589.28278 | 295.14503 | 574.27188 | 575.27971 | 288.14349 | 287.63958 | 573.26406 | 287.13567 | 5 |
| 492.23002 | 246.61865 | 477.21912 | 478.22694 | 239.61711 | 239.11320 | 476.21129 | 238.60929 | 4 |
| 377.20308 | 189.10518 | 362.19218 | 363.20000 | 182.10364 | 181.59973 | 361.18435 | 181.09581 | 3 |
| 276.15540 | 138.58134 | 261.14450 | 262.15232 | 131.57980 | 131.07589 | 260.13667 | 130.57197 | 2 |
| 147.11280 | 74.06004 | 132.10191 | 133.10973 | 67.05850 | 66.55459 | 131.09408 | 66.05068 | 1 |

TABLE 13
Fragment ion table and spectrum of peptide SLHTLFGDELCK
(SEQ. ID. NO: 4), obtained from HCD fragmentation.

| | | | | Internal |
|---|---|---|---|---|
| | Ion Series | Neutral Losses | Precursor Ions | Fragments |
| #1 | b⁺ | Seq. | y⁺ | #2 |
| 1 | 184.08765 | S-PC bioti . . . | | 12 |
| 2 | 297.17172 | L | 1332.66165 | 11 |
| 3 | 434.23063 | H | 1219.57758 | 10 |
| 4 | 535.27831 | T | 1082.51867 | 9 |
| 5 | 648.36237 | L | 981.47099 | 8 |
| 6 | 795.43079 | F | 868.38693 | 7 |
| 7 | 852.45225 | G | 721.31852 | 6 |
| 8 | 967.47919 | D | 664.29705 | 5 |
| 9 | 1096.52179 | E | 549.27011 | 4 |
| 10 | 1209.60585 | L | 420.22752 | 3 |
| 11 | 1369.63650 | C-Carbami . . . | 307.14345 | 2 |
| 12 | | K | 147.11280 | 1 |

TABLE 14
Fragment ion table peptide SLHTLFGDELCK (SEQ. ID. NO: 4), obtained from
EThcD fragmentation (ETD with supplemental HCD activation).
Ion Series | Neutral Losses | Precursor Ions | Internal Fragments

| #1 | c⁺ | c⁺H⁺ | c⁻H⁺ | Seq. | y⁺ | z⁺ | z⁺H⁺ | z⁻H⁺ | #2 |
|---|---|---|---|---|---|---|---|---|---|
| 1 | 105.06585 | 106.07368 | 104.05803 | S | | | | | 12 |
| 2 | 218.14992 | 219.15774 | 217.14209 | L | 1428.71000 | 1413.69910 | 1414.70692 | 1412.69127 | 11 |
| 3 | 451.25718 | 452.26500 | 450.24935 | H-PC bioti . . . | 1315.62593 | 1300.61504 | 1301.62286 | 1299.60721 | 10 |
| 4 | 552.30486 | 553.31268 | 551.29703 | T | 1082.51867 | 1067.50777 | 1068.51560 | 1066.49995 | 9 |
| 5 | 665.38892 | 666.39675 | 664.38110 | L | 981.47099 | 966.46010 | 967.46792 | 965.45227 | 8 |
| 6 | 812.45734 | 813.46516 | 811.44951 | F | 721.31852 | 853.37603 | 854.38386 | 852.36821 | 7 |
| 7 | 869.47880 | 870.48662 | 868.47097 | G | 664.29705 | 706.30762 | 707.31544 | 705.29979 | 6 |
| 8 | 984.50574 | 985.51357 | 983.49792 | D | 549.27011 | 649.28615 | 650.29398 | 648.27833 | 5 |
| 9 | 1113.54834 | 1114.55616 | 1112.54051 | E | 420.22752 | 534.25921 | 535.26704 | 533.25139 | 4 |
| 10 | 1226.63240 | 1227.64022 | 1225.62457 | L | 307.14345 | 405.21662 | 406.22444 | 404.20879 | 3 |
| 11 | 1386.66305 | 1387.67087 | 1385.65522 | C-Carbami . . . | 147.11280 | 292.13255 | 293.14038 | 291.12472 | 2 |
| 12 | | | | K | | 132.10191 | 133.10973 | 131.09408 | 1 |

We observed azidylation on multiple residues; modification of histidine, lysine, tryptophan, tyrosine, and isoleucine was confirmed using diagnostic fragment ions in tandem mass spectra (see Table 15).

TABLE 15
Azidylation increases significantly with increased azide dose in the
presence of hydrogen peroxide.

| | | | # of peptide spectral matches | | | | | |
|---|---|---|---|---|---|---|---|---|
| | | | No H2O2 | | | 1% H2O2 | | |
| | Residue | Site of | 1 mM | 10 mM | 100 mM | 1 mM | 10 mM | 100 mM |
| Peptide(s) | #s | Modification | Azide | Azide | Azide | Azide | Azide | Azide |
| CELAAAMKR | 6-14 | K13 | — | — | — | — | — | 40 |
| (SEQ ID NO: 18) | | | | | | | | |
| RHGLONYR | 14-21 | H15 | — | — | — | — | — | 111 |
| (SEQ ID NO: 38) | | | | | | | | |
| GYSLGNWVCAAK | 22-33 | W28 | — | — | — | — | 25 | 58 |
| (SEQ ID NO: 39) | | | | | | | | |
| NTDGSTDYGILOINSR | 46-61 | Y53 | — | — | — | — | — | 57 |
| (SEQ ID NO: 40) | | | | | | | | |
| NLCNIPCSALLSSDITAS | 74-96 | 188 | — | 13 | 31 | — | 2 | 52 |
| VNCAK (SEQ ID NO: 41) | | | | | | | | |
| IVSDGNGMNAWVAWR | 98-112 | A110/ | — | — | — | — | 25 | 63 |
| (SEQ ID NO: 20) | | W111 | | | | | | |
| GTDVQAWIR (SEQ ID NO: 21) | 117-125 | W123 | — | — | — | 49 | 92 | 118 |
| | | Total PSMs -> | 0 | 13 | 31 | 49 | 144 | 459 |

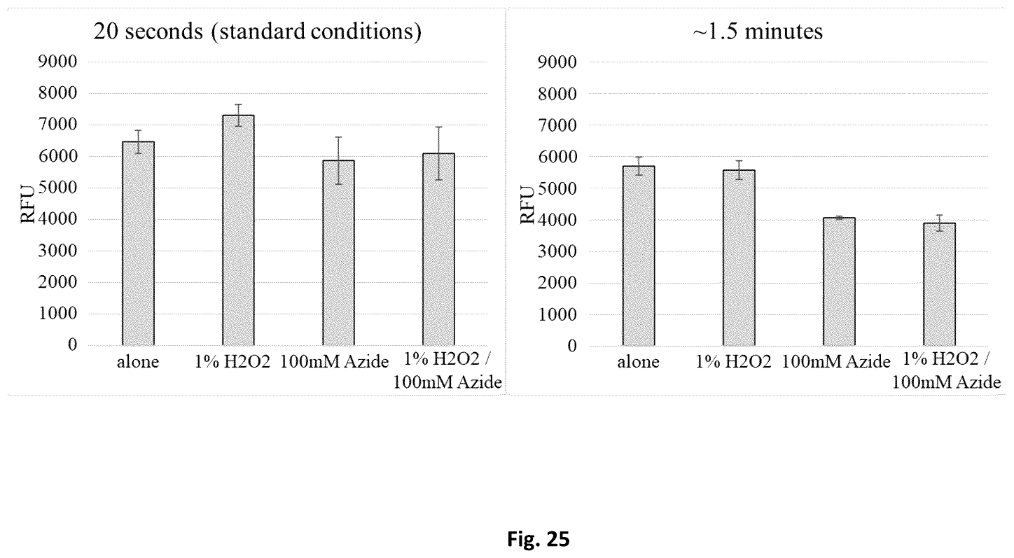

Observing lysine and histidine modification suggests that charge-based coordination could play some role, in that the azide anion may be interacting with positively charged surface residues; however, mapping the modified residues to the structure revealed that although the charged residues are partially surface exposed, the uncharged ones are buried within an internal cleft sandwiched by helices (FIG. 24C), a phenomenon we address in the Discussion. Surprisingly, we observed azide dose-dependent azidylation at residue 189 in the absence of added hydrogen peroxide, which as described below, can be explained by oxidation during the subsequent click reaction. This was the only residue that displayed such behavior and adding hydrogen peroxide did not increase its azidylation significantly. All other sites of modification were only identified with added hydrogen peroxide. Samples supplied with 100 mM azide exhibited far more azidylation than those with lower doses—to verify that this combined treatment with H₂O₂ and azide isn't denaturing, we analyzed GFP fluorescence under identical reaction conditions and found that no fluorescence was lost, suggesting that the conditions used are nondenaturing. However, letting the reaction continue for 60 seconds beyond the initial 20 second treatment led to minor loss of fluorescence in azide-containing samples, and thus it is likely that while longer treatments cause unfolding (FIG. 25), the conditions utilized here are benign and do not cause denaturation.

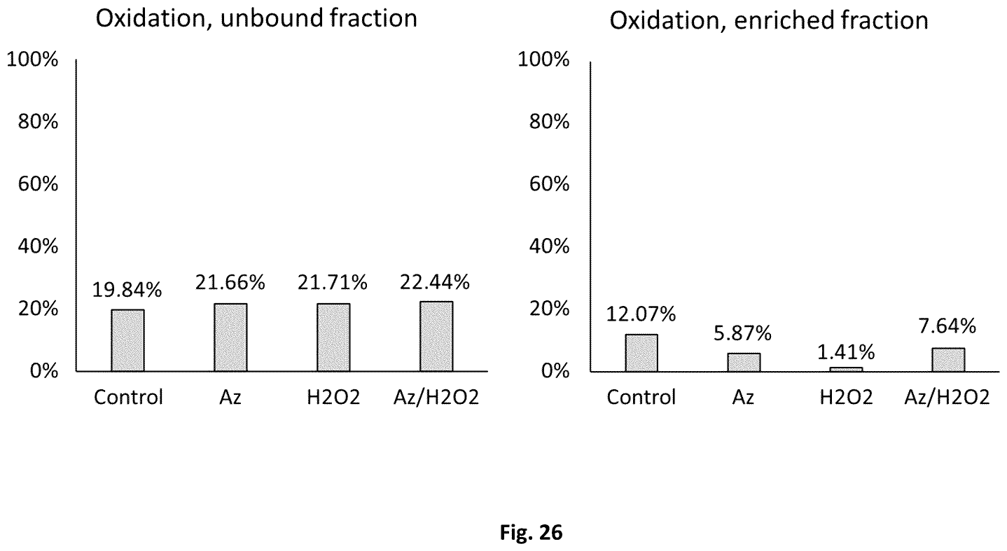

Though the atomic composition of the azidylation product was verified with high resolution mass spectrometry, the precise mechanism for oxidative azidylation is unknown and remains an active area of study. That said, the simplest explanation for the observed modification in this first study using folded soluble proteins is that azide radical attack displaces a bound hydrogen, after which clicking and photocleaving forms the +96.04 adduct, a mass shift that is inconsistent with addition of azide across a double bond, but rather is only consistent with free radical attack. Furthermore, significant residue level oxidation or free radical-induced protein crosslinking were not observed (FIGS. 26 and 24A), suggesting side chain oxidation intermediates are not involved in the reaction. Thus, we hypothesize that the reaction proceeds via H₂O₂-induced azido radical attack of the modified residues at an available carbon, instigating a loss of hydrogen to maintain carbon's stable bonding pattern.

### BSA Noncovalently Binds Azide to Enable ‘One-Pot’ Azidylation and Click Chemistry:

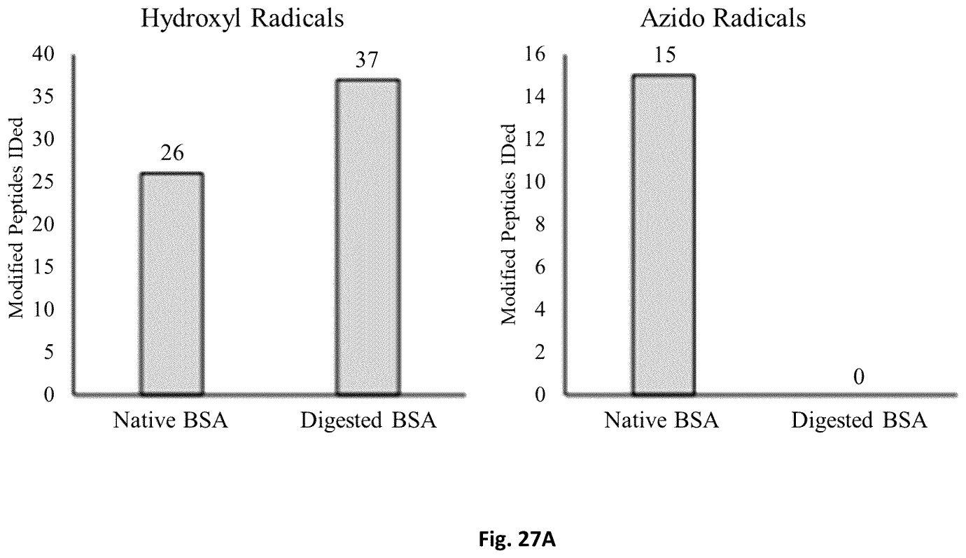

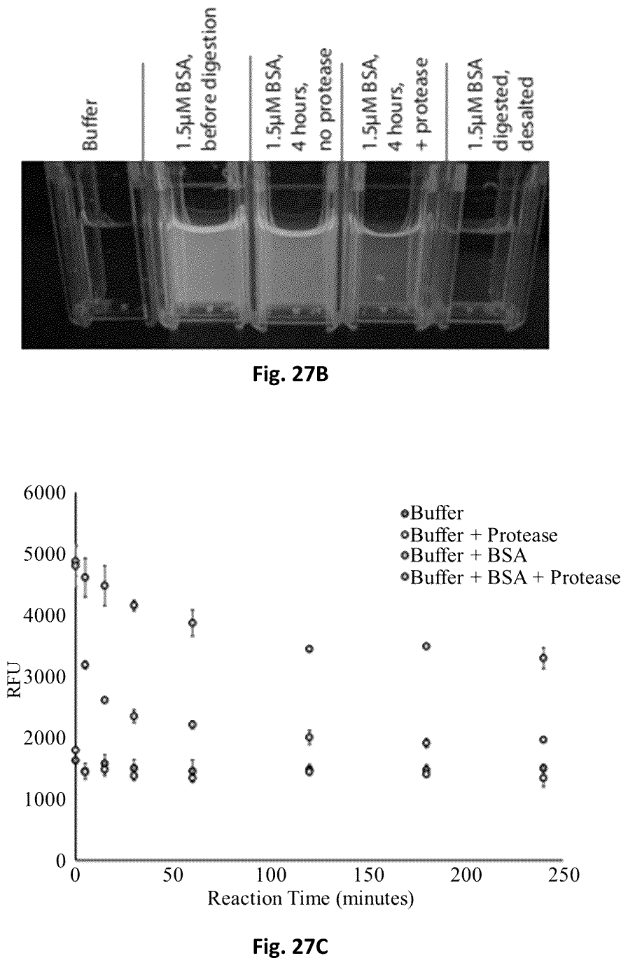

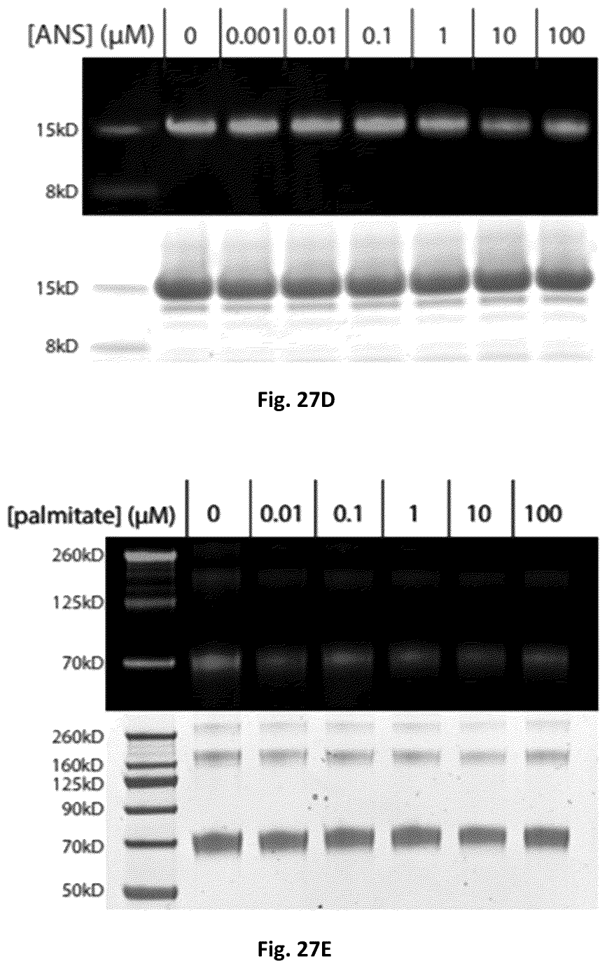

To test the azido radical's ability to modify other proteins and further investigate the phenomenon of H₂O₂-independent azidylation, bovine serum albumin (BSA) was used. As shown in FIGS. 27A to 27E, BSA azidylation was detected using both blotting and MS. H₂O₂ addition alone did not lead to significant modification (FIG. 24D) or oxidation above control levels (FIG. 26). As observed with lysozyme, addition of 100 mM azide alone to BSA led to significant azidylation. The effect was clearly azide dose-dependent and stronger than with lysozyme (FIG. 24D—at 100 mM supplied azide, levels of azidylation were virtually identical regardless of added hydrogen peroxide. At 10 mM azide, slightly less azidylation was observed in the H₂O₂ containing samples, and at 1 mM azide, significantly less azidylation was observed when H₂O₂ wasn't added. Thus, the phenomenon observed with lysozyme was also observed with BSA—at lower azide levels, H₂O₂ is necessary for BSA azidylation, whereas at higher azide concentrations, detectable azidylation is observed without added hydrogen peroxide. On BSA, the major sites of modification were histidine and serine, though the two azidylated serines were next to histidines (see Table 16).

TABLE 16
Fragment ion table (peptide = BSA) showing azidylations without added hydrogen
peroxide.

| | | | #PSMS | | | | | |
|---|---|---|---|---|---|---|---|---|
| | | | no H2O2 | | | 1% H2Os | | |
| | Residues | Site of | 1 mM | 10 mM | 100 mM | 1 mM | 10 mM | 100 mM |
| Peptide(s) | in protein | Modification | Azide | Azide | Azide | Azide | Azide | Azide |
| FKDLGEEHFK | 35-44 | H42 | — | 40 | 124 | — | 7 | 115 |
| (SEQ. ID. NO: 1) | | | | | | | | |
| DLGEEHFK (SEQ. ID. NO: 2) | 37-44 | | | | | | | |
| TCVADESHAGCEK | 76-88 | H83 | — | — | 15 | — | — | 43 |
| (SEQ. ID. NO: 3) | | | | | | | | |
| SLHTLFGDELCK | 89-100 | S89 | — | 62 | 170 | — | — | 170 |
| (SEQ. ID. NO: 4) | | | | | | | | |
| QEPERNECFLSHKDDSPDLPK | 118-138 | H129 | — | — | 51 | — | — | 124 |
| (SEQ. ID. NO: 5) | | | | | | | | |
| NECFLSHKDDSPDLPK | 123-138 | | | | | | | |
| (SEQ. ID. NO: 42) | | | | | | | | |
| ECCHGOLLECADDRADLAK | 267-285 | H270 | — | — | 19 | — | — | 21 |
| (SEQ. ID. NO: 7) | | | | | | | | |
| VHKECCHGDLLECADDRAD | 264-285 | | — | — | 11 | — | — | 36 |
| LAK (SEQ. ID. NO: 8) | | | | | | | | |
| SHCIAEVEK | 310-318 | S310 | 16 | 40 | 197 | 22 | 93 | 380 |
| (SEQ. ID. NO: 9) | | | | | | | | |
| SHCIAEVEKDAIPENLPPLTA | 310-340 | | | | | | | |
| DFAEDKDVCK | | | | | | | | |
| (SEQ. ID. NO: 10) | | | | | | | | |
| DDPHACYSTVEDK | 387-399 | H390 | — | — | 42 | — | 6 | 96 |
| (SEQ. ID. NO: 32) | | | | | | | | |
| EYEATLEECCAKDOPHACYS | 375-399 | | | | | | | |
| TVEDK (SEQ. ID. NO: 43) | | | | | | | | |
| LKHLVDEPONLIK | 400-412 | H403 | — | 6 | 212 | | — | 253 |
| (SEQ. ID. NO: 13) | | | | | | | | |
| HLVDEPONLIK | 402-412 | | | | | | | |
| (SEQ. ID. NO: 14) | | | | | | | | |
| LFTFHADICTLPOTEK | 529-544 | H533 | — | — | 91 | — | — | 105 |
| (SEQ. ID. NO: 15) | | | | | | | | |

The repeated phenomenon of H₂O₂-independent azidylation led to the hypothesis that lysozyme and BSA are noncovalently binding azide (either the anion, ⁻N₃ or the protonated anion, HN₃) in solution. We hypothesized that this binding is so tight that with BSA, at a minimum of 1 mM azide, some azide remains bound following a round of trichloroacetic acid precipitation and two straight acetone washes performed prior to the click reaction, our standard protocol. We thus asked whether small amounts of added azide are sufficient for concurrent BSA azidylation and clicking in the CuAAC reaction. If so, this is consistent with BSA binding and carrying azide through precipitation at sufficient levels to observe the azidylation seen in the absence of H₂O₂. This was indeed the case; as low as 100 μM azide added to a click reaction containing BSA that had never previously seen azide caused detectable azidylation. See Table 17.

TABLE 17
Modified residue table table showing that performing the one-pot
azidylation and CuAAC reactions leads to more azidylation, on
regions and residues not modified on native BSA

| | | | #PSMs | | | |
|---|---|---|---|---|---|---|
| | | Site of | 10 uM | 100 uM | 1 mM | 10 mM |
| Peptide(s) | Residues | Modification | Azide | Azide | Azide | Azide |
| SEIAHRFKDLGEEHFK | 29-44 | H33 | — | — | — | 62 |
| (SEQ. ID. NO: 22) | | | | | | |
| FKDLGEEFHK (SEQ. ID. NO: 23) | 35-44 | H42 | — | 20 | 111 | 194 |
| GLVLIAFSQYLQQCPFDEHVK | 45-65 | Unable to | — | — | — | 58 |
| (SEQ. ID. NO: 24) | | localize | | | | |
| LVNELTEFAK (SEQ. ID. NO: 25) | 66-75 | L66/V67 | — | — | — | 21 |
| TCVADESHAGCEK (SEQ. ID. NO: 3) | 76-88 | H83 | — | — | — | 29 |
| SLHTLFGDELCK (SEQ. ID. NO: 4) | 89-100 | S89 | — | 53 | 96 | 102 |
| ETYGDMADCCEK (SEQ. ID. NO: 26) | 106-117 | Unable to | — | — | — | 6 |
| | | localize | | | | |
| QEPERNECFLSHKDDSPDLPK | 118-138 | H129 | — | — | 71 | 122 |
| (SEQ. ID. NO: 5) | | | | | | |
| AEFVEVTKLVTDLTK | 249-263 | K256 | — | — | — | 13 |
| (SEQ. ID. NO: 27) | | | | | | |
| ECCHGDLLECADDRADLAK | 267-285 | H270 | — | 4 | 104 | 197 |
| (SEQ. ID. NO: 28) | | | | | | |
| VHKECCHGDLLECADDRADLAK | 264-285 | | | | | |
| (SEQ. ID. NO: 8) | | | | | | |
| SCHIAEVEK (SEQ. ID. NO: 29) | 310-318 | S310 | — | 51 | 251 | 216 |
| SCHIAEVEKDAIPENLPPLTADFAEDK | 310-340 | | | | | |
| DVCK (SEQ. ID. NO: 30) | | | | | | |
| DAFLGSFLYEYSR (SEQ. ID. NO: 31) | 347-359 | Y355/357 | — | — | 11 | 56 |
| DDPHACYSTVFDK (SEQ. ID. NO: 32) | 387-399 | H390 | — | 5 | 56 | 36 |
| EYEATLEECCAKDDPHACYSTVEDK | 375-399 | | | | | |
| (SEQ. ID. NO: 12) | | | | | | |
| DDPHACYSTVFDKHKLHVDEPQNLIK | 387-412 | H403 | — | 99 | 199 | 324 |
| (SEQ. ID. NO: 33) | | | | | | |
| LKHLVDEPQNLIK (SEQ. ID. NO: 16) | 400-412 | | | | | |
| HLVDEPQNLIK (SEQ. ID. NO: 34) | 402-412 | | | | | |
| MPCTEDYLSLILNR (SEQ. ID. NO: 35) | 469-482 | Unable to | — | — | — | 4 |
| | | localize | | | | |
| AFDEKLFTFHADICTLPDTEK | 524-544 | K529 | — | — | 39 | 75 |
| (SEQ. ID. NO: 36) | | | | | | |
| LFTFHADICTLPDTEK | 529-544 | H533 | — | 74 | 119 | 106 |
| (SEQ. ID. NO: 17) | | | | | | |

MS analysis confirmed that the modification is the identical +96.04 amu mass shift observed following photocleavage of H₂O₂-mediated azidylation as well, suggesting that this addition proceeds via the same mechanism. Additionally, we observed modification in regions and on residues not seen when azidylation occurred in PBS and with H₂O₂, rather than in 4M urea and with the CuAAC reagents. For example, as with lysozyme, a modified lysine was also observed on BSA, as well as a peptide containing either a modified leucine or valine (data not shown). We take this increased azidylation to be a result of urea-based alterations in the 3D structure and dynamic intramolecular motion of the protein in solution. We hypothesize that this ‘one-pot clicking’ phenomenon is due to the oxidative nature of the CuAAC reaction combined with BSA's clear proclivity to bind azide. Altogether, the data suggest that BSA is binding azide noncovalently with high affinity, thereby supplying it to the click reaction and enabling the observed ‘one-pot’ clicking.

Oxidative Azidylation in a Cell-Free Lysate of Arabidopsis thaliana Identifies Known Azide Binding Sites:

Given the above observations, we hypothesized that performing oxidative azidylation on a complex mixture would yield covalent modification on known azide-binding proteins. To test this, we azidylated an Arabidopsis thaliana soluble proteome containing metabolites and buffer components, i.e., conditions different from the two pure model proteins which were simply dissolved in PBS. In contrast to the two pure proteins, we found that 400 mM azide, an acid cleavable biotin alkyne (FIG. 23C), and acquiring mass spectra with HCD fragmentation provided the best survey of azidylated proteins. From this analysis, we identified 55 peptides containing the +125.06 amu mass adduct corresponding to azidylation from a range of proteins (data not shown).

The three most azidylated proteins observed in the lysate were the large chain subunit of rubisco, catalase, and Cu/Zn superoxide dismutase (data not shown). On rubisco, seven peptides containing azidylation were found. Catalase is well-known to be inhibited by azide binding (40, 41). Our experiments corroborated this and demonstrated that the cytosol was still catalytically active since when H₂O₂ was added to our samples, native catalase quickly broke it down, resulting in significant foaming. Addition of azide prior to H₂O₂ prevented this entirely. Thus, it was not surprising to observe four sites of azidylation on catalase, consistent with its known azide binding ability. The third major azidylated protein observed was Cu/Zn superoxide dismutase, another known azide-binding protein. As with catalase, Cu/Zn superoxide dismutases are inhibited by azide binding (42). One site of azidylation was identified on Cu/Zn superoxide dismutase, V123. Minor azidylation was also identified on a handful of other proteins, for which no obvious commonality in terms of gene ontology or sequence motif enrichment was present. These included the azide-binding protein cytochrome C, the H₂O₂-removal enzymes peroxidase 38 and L-ascorbate peroxidase, a few transporters (presumably arising from small microsomes not removed by the centrifugation employed), and an array of other metabolic synthesis proteins.

Overall, azidylating a cell-free tissue lysate comprised of unfractionated, soluble Arabidopsis proteins demonstrated that azidylation occurring in a complex protein background and a different buffer system from PBS could indeed also be identified, and that some of the proteins most readily observed are already known to be azide-binding. Thus, radical-mediated azidylation in a complex molecular background corroborated previously observed noncovalent azide binding. We have not yet scaled the clicking and capture reaction sufficiently to reveal additional azidylated proteins nor have we examined intracellular compartments other than a clarified cell-free lysate. Thus, other well-known azide binding proteins such as the F1-ATPase were not observed (37). We hypothesize that the reported azidylation protocol is applicable to many, if not all, proteins.

### Azidylation Requires 3D Structure and Can be Outcompeted by Hydrophobic Ligands:

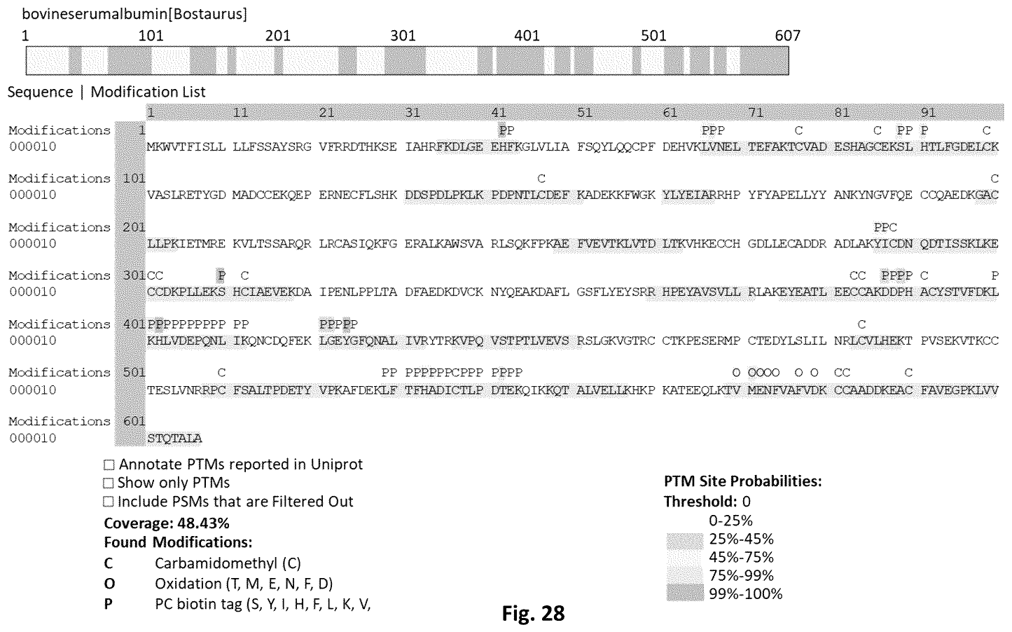

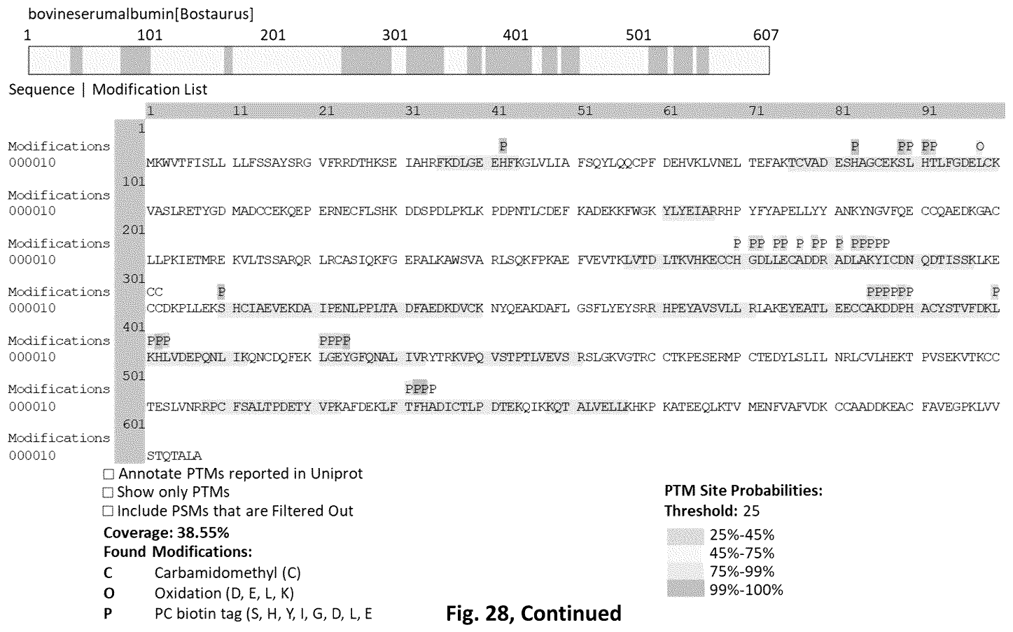

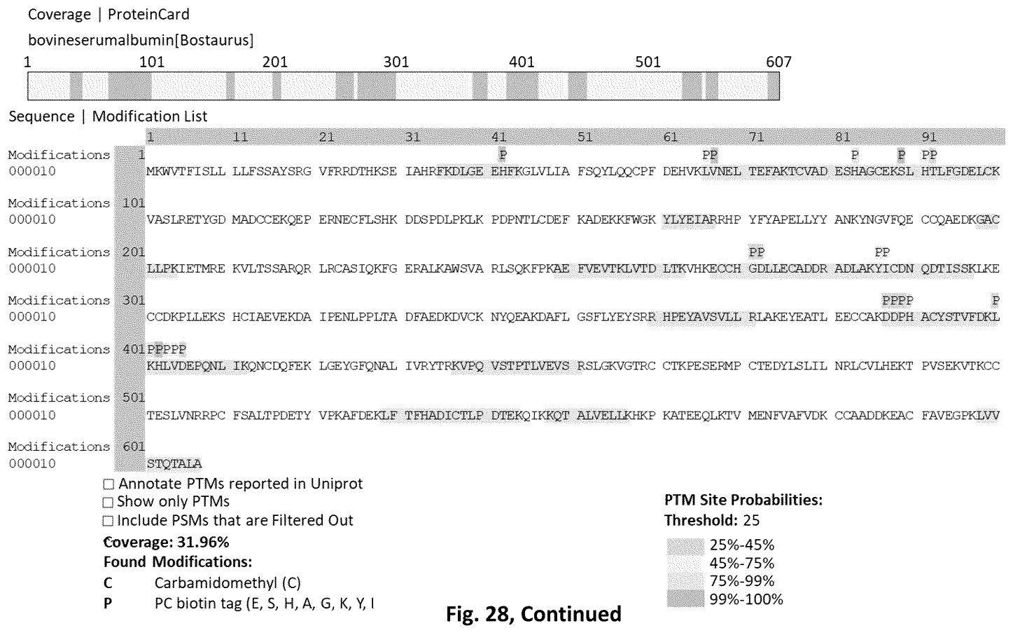

We next considered what principles could drive protein:azide binding, and noted that azido radical labelling, unlike hydroxyl radical modification, does not depend upon solvent accessibility. Previously, BSA pre-digested into peptides prior to modification demonstrated significantly more hydroxyl radical labeling in many more sites than structured, native BSA in solution, suggesting solvent accessibility is a major factor mediating OH radical labeling (21). When an analogous experiment was done with azidylation, no modification was observed on digested peptide samples across multiple replicates, and native BSA concurrently azidylated with the same solvents exhibited expected levels of azidylation (FIGS. 27A, 28, and 29). This result is striking, given the opposite result with hydroxyl radical modification, which depends on solvent accessibility rather than inaccessibility.

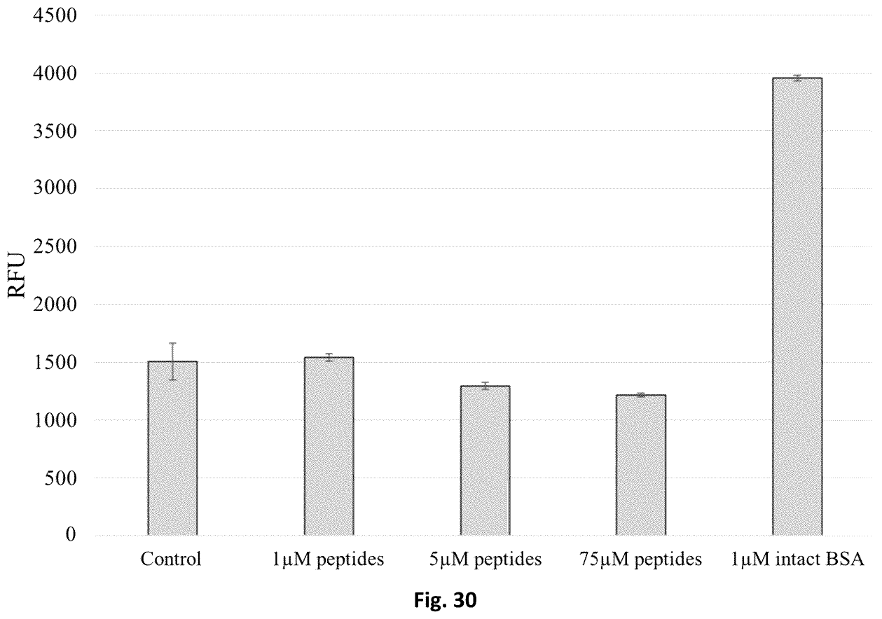

We thus asked whether the fluorescent probe of hydrophobicity in folded proteins, 8-anilinonaphthalene-1-sulfonic acid (ANS), exhibits similar behavior to azide. This was the case. As full-length BSA is digested to peptides with proteases, we found that ANS fluorescence significantly decreases over time, and that completely digested, desalted BSA peptides, even when supplied in higher concentrations than native BSA, cause no detectable ANS fluorescence (FIGS. 27B, 27C, and 30). We next asked whether ANS competes for azide binding and in turn inhibits azidylation. It did-we observed that ANS inhibited lysozyme azidylation in a dose-dependent fashion as measured both by blotting and MS (FIG. 27D).

In order to test whether other hydrophobic ligands would prevent azidylation, we examined the fatty acid: BSA interaction. Serum albumins have a significant role in lipid biology-in blood, they bind fatty acids of many types with high affinity via noncovalent hydrophobic interaction (22-24). We found that palmitate added to the azidylation reaction inhibited azidylation at concentrations as low as 10 nM (FIG. 27E), consistent with a model in which palmitate binds to BSA with high affinity and sterically blocks azidylation. Overall, unlike hydroxyl radical labeling, three-dimensional structure is necessary for both azidylation and ANS fluorescence and supplied hydrophobic ligands such as ANS or fatty acid can outcompete azidylation.

### DISCUSSION

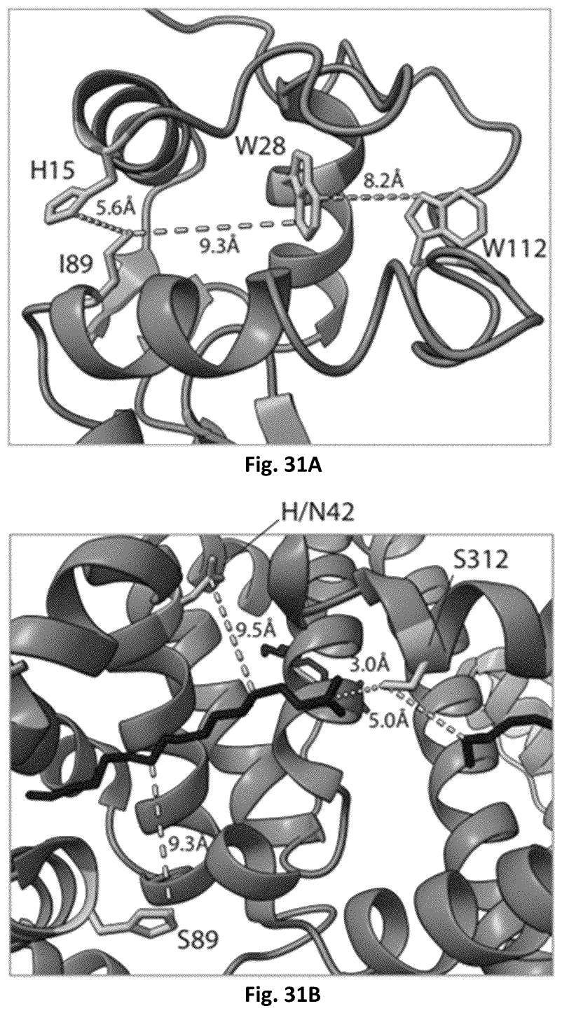

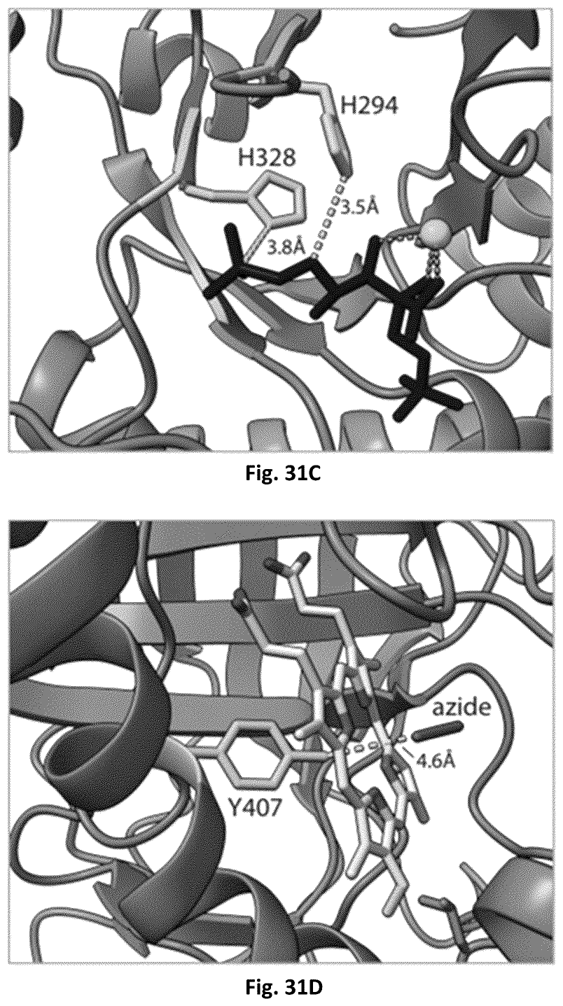

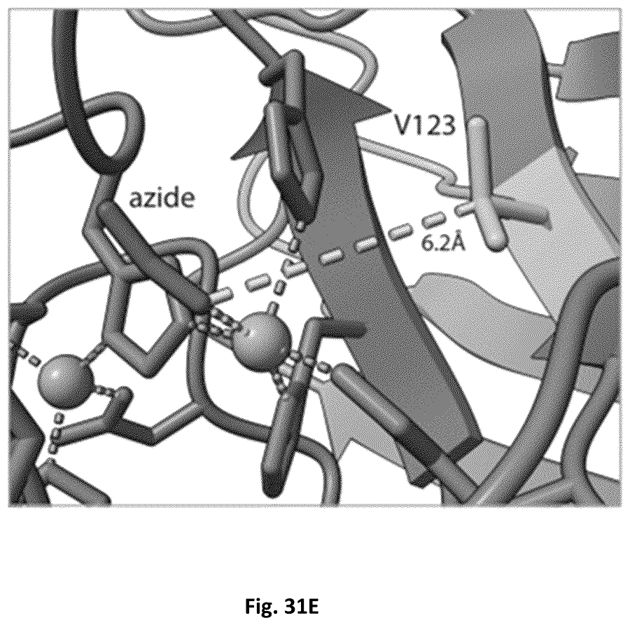

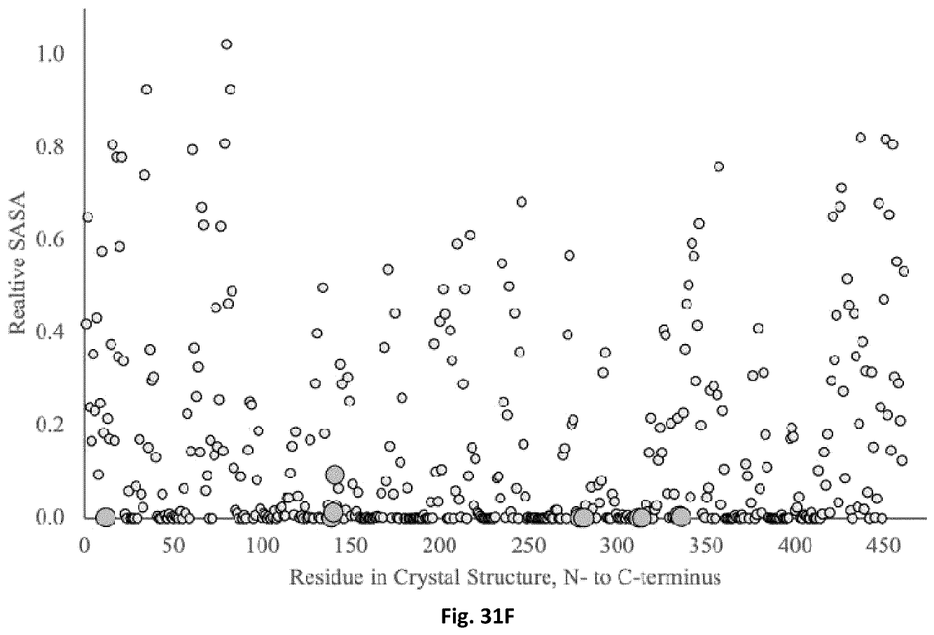

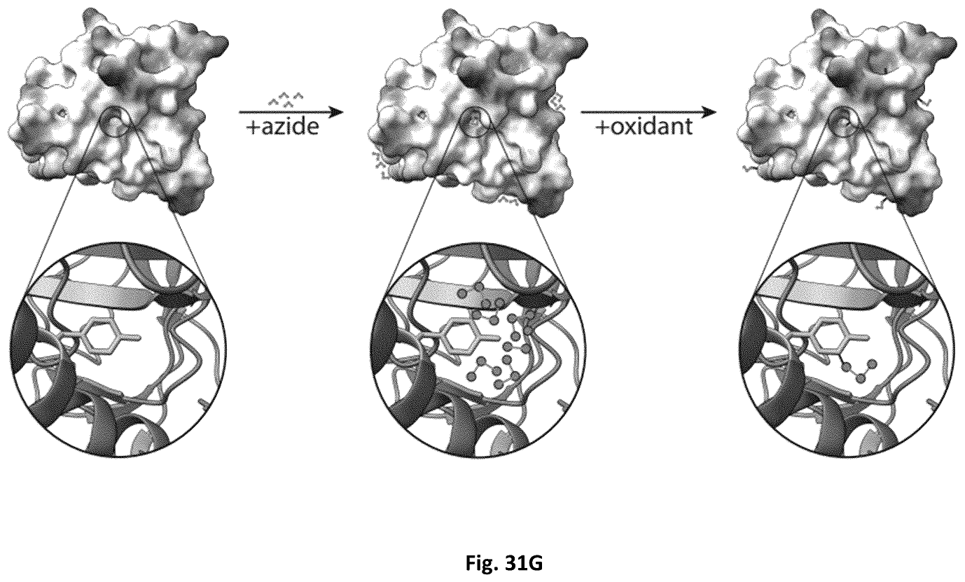

In this Example, we have described a method for covalently modifying protein with the azido radical, which can be used to capture and identify locations of noncovalent azide binding driven by three-dimensional hydrophobic forces. On lysozyme, seven residues were azidylated-K13, H15, W28, Y53, 189, A111/W112 (which we hypothesize is in fact W112, but MS/MS did not conclusively localize this site), and W123. A crystal structure-based hydrophobicity detection method predicts that 189 is in direct proximity to a major known hydrophobic patch (25). More strikingly, H15, W28, 189, and W112 are within a contiguous buried cleft sandwiched by three alpha helices and with their side chains pointed inward and aligned (FIG. 31A). Calculating solvent accessibility from the crystal structure demonstrated these residues are among the least solvent accessible in the whole protein, and clearly the group of modified residues in total trends strongly toward solvent inaccessible (FIG. 32) (43).

BSA, more so than lysozyme, was strongly modified in the absence of H₂O₂ but the presence of azide (FIG. 24D), and this azidylation could be outcompeted by adding one of BSA's native hydrophobic ligands, palmitate. As shown in the tables, in BSA, azide dose-dependent azidylation occurs mainly on histidines or proximal to histidines on serines and is unequally distributed. Peptide spectral match (PSM) counts are residue-centric, i.e., missed cleavage events that led to azidylation on specified residues had their PSMs summed with fully cleaved peptides that contain the same azidylation site. (Data not shown.) A structure for BSA co-crystallized with palmitate does not exist; however, human serum albumin (HSA), which exhibits significant sequence and structural similarity to BSA, has been crystallized with bound palmitate (23, 24). Aligning the sequences and mapping sites that are azidylated in BSA to the HSA: palmitate structure revealed that three of the five sites that are azidylated with 10 mM azide, which we take to be higher affinity binding sites, are within 11 Å of a pocket in HSA that binds two palmitate molecules, and the only residue azidylated with 1 mM azide, S310, conserved in HSA as S312, is within 5.0 Å of each palmitate (FIG. 31B). When azidylation is performed concurrently with CuAAC in a ‘one-pot’ 4M urea-containing reaction, more regions were labelled than in native BSA, a result that was not seen when using digested BSA (data not shown). This contrast has led us to hypothesize that as urea alters the 3D structure of a protein, the process creates more hydrophobic pockets for azide binding, consistent with the idea that urea-mediated protein changes in intramolecular dynamics occur via hydrophobic patch solvation (26, 27).

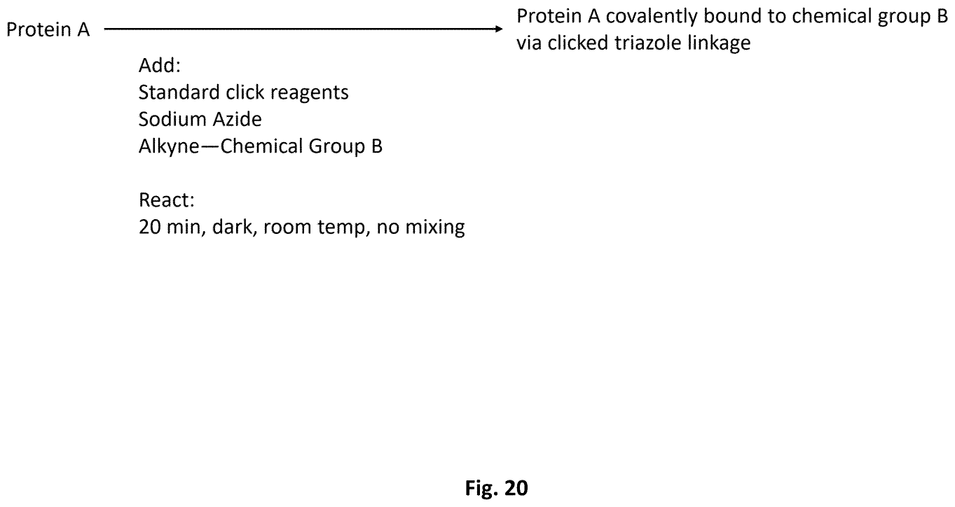

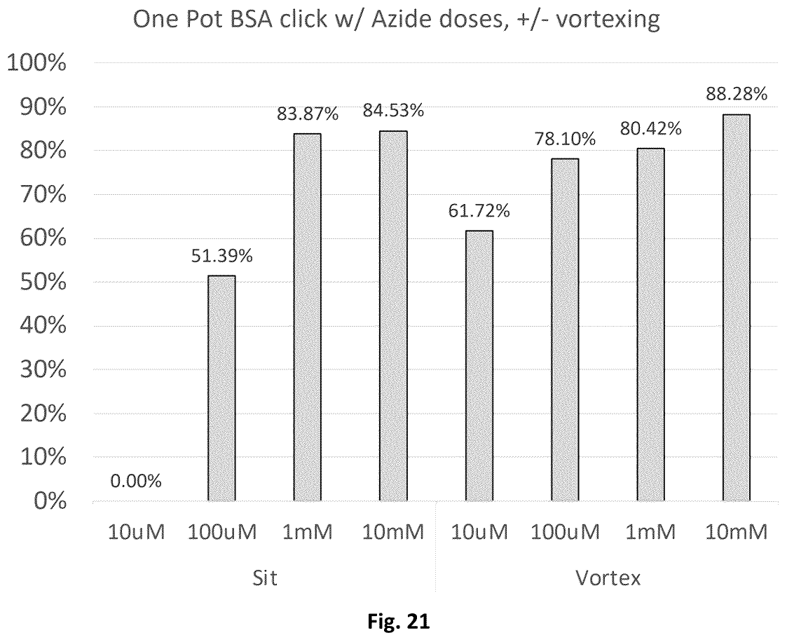

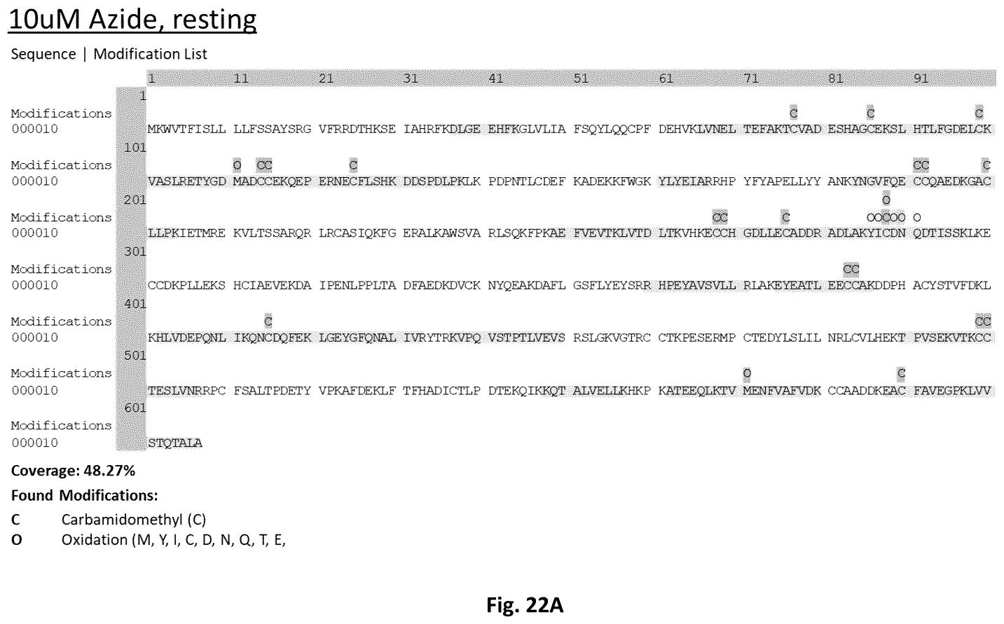

Both BSA and lysozyme's interactions with the fluorescent probe ANS have been studied (28, 44-46). That said, there isn't consensus on the precise sites of interaction and the vast majority of work details protein conformational shifting, unspecified sites of high- and low-affinity ANS binding, and in the case of lysozyme, fibrillation and inhibition thereof (28, 44-46). We reasoned that if protein:azide complex formation occurs within hydrophobic patches, then ANS should compete for azide binding. Indeed, we found that added ANS decreases azidylation in a dose-dependent fashion as measured using both streptavidin blotting and mass spectrometry (FIG. 27D). Moreover, digesting BSA into peptides abolished both ANS fluorescence and azidylation (FIGS. 18A-27B). Our interpretation of these observations is that three-dimensional structure is necessary for noncovalent azide and ANS binding and covalent azidylation. These results, which are in direct contrast to the effect predigesting protein has on hydroxyl radical modification, led to consideration of fundamental differences between hydroxyl and azido radicals. Since the azido radical has delocalized, resonant pi electrons and the most azidylated residues are aromatic (histidine in BSA and tryptophan in lysozyme), we reasoned that there might be an affinity between azide and aromatic residues. If this were the full explanation, however, then predigested BSA would exhibit a similar, or at least some, level of modification as the native protein, which was not observed, and we would expect that labelling would be restricted to aromatic residues, which is not the case. (See Tables 9-15 hereinabove) Thus, our interpretation is that azide is acting as an ‘affinity reagent’ for structured hydrophobic patches, and via oxidation, can covalently modify amino acids within or near three-dimensional hydrophobic patches.

The strongest azidylation identified from an Arabidopsis lysate occurred on rubisco, catalase, and Zn/Cu superoxide dismutase. See Table 18.

TABLE 18
The majority of detectable azidylation in Arabidopsis lysate i;
concentrated on RuBisCO, Catalase, and superoxide dismutase.

| Protein/ | | Azidylated | |
|---|---|---|---|
| Uniprot | Peptide Identified | Residues | PSMs |
| Catalase 3 | VPTPTNSYTGER (SEQ ID. NO: 44) | Not localized | 8 |
| A0A1P8AWT7 | IFAYGDTQR (SEQ ID. NO: 45) | Y407 | 13 |
| | LGPNYLQLPVNAPK (SEQ ID. NO: 46) | Y419 | 17 |
| | TNIQEYWR (SEQ ID. NO: 47) | Y228 | 7 |
| RuBisCo | ELGVPIVMHDYLTGGFTANTSLSHYCR | Not localized | 5 |
| Large | (SEQ ID. NO: 48) | | |
| Subunit | YGRPLLGCTIKPK (SEQ ID. NO: 49) | Not localized | 2 |
| O03042 | LTYYTPEYETK (SEQ ID. NO: 50) | Y24 | 12 |
| | TFQGPPHGIQVER (SEQ ID. NO: 51) | P151, P152, | 16 |
| | | H153 | |
| | DNGLLLHIHR (SEQ ID. NO: 52) | H292, H294 | 39 |
| | LSGGDHIHAGTVVGK (SEQ ID. NO: 53) | H326, H328 | 76 |
| | ESTLGFVDLLR (SEQ ID. NO: 54) | L348, L349 | 16 |
| Superoxide | AVVVHADPDDLGK (SEQ ID. NO: 55) | V123 | 11 |
| Dismutase | | | |
| [Cu/Zn] | | | |
| 3/q9Fk60 | | | |

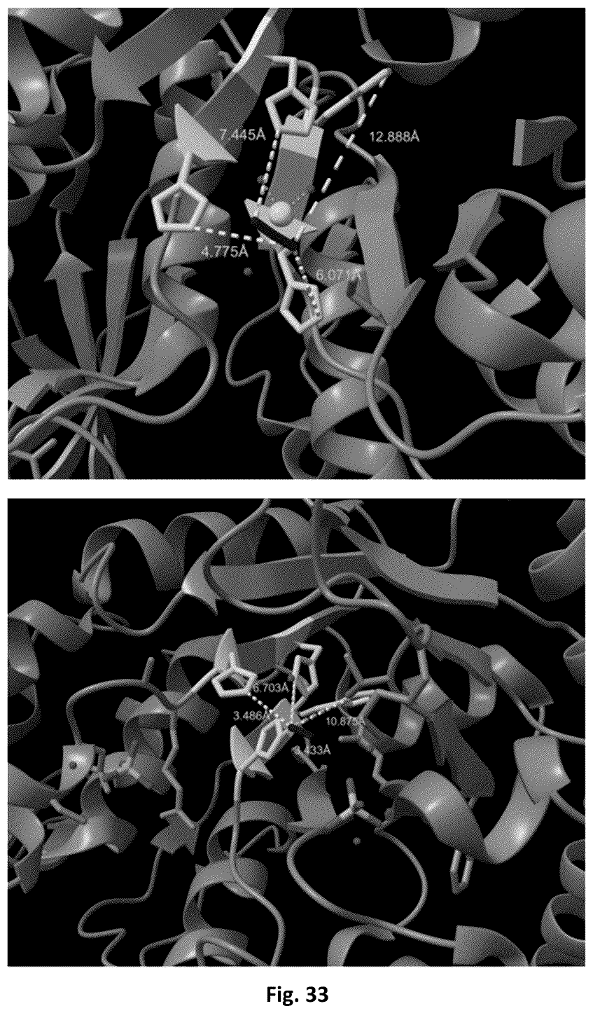

The two major regions of azidylation identified on rubisco, H326/H328 and H292/H294, are adjacent to the catalytic site, and H294 and H328 are both within 6 Å of the bound transition state analogue 2-carboxyarabinitol-1,5-diphosphate in a recent crystal structure (FIG. 31C) (47). These four histidines are conserved between Arabidopsis and Galdieria rubisco, and are within ˜10 Å of both CO₂ and O₂ cocrystallized in the respective crystal structures (FIG. 33) (48). Multiple modified residues and regions, including the above, are spatially close (˜15 Å apart at most) and suggest that azide may cluster within and in close proximity to the catalytic site. On catalase, three of the four sites were localized to Y228, Y407, and Y419. Of these, both Y407 and Y419 are conserved in Bos taurus catalase, for which a crystal structure with bound azide has been solved (49). Examining the structure revealed that Y407, one of the major sites of azidylation identified here, directly coordinates the heme cofactor that interacts with bound azide (and H₂O₂, during catalysis), and azide is less than 5 Å from Y407 (FIG. 31D). The second conserved site, Y419, is ˜11 Å behind Y407. On Cu/Zn superoxide dismutase, only V123 was azidylated. This valine is conserved across multiple genera (50) and mapping it on a crystal structure of Saccharomyces cerevisiae Cu/Zn superoxide dismutase with bound azide revealed that it is directly proximal to the site of Cu coordination, pointed into the catalytic site, and ˜6 Å from the bound azide (FIG. 31E) (42).

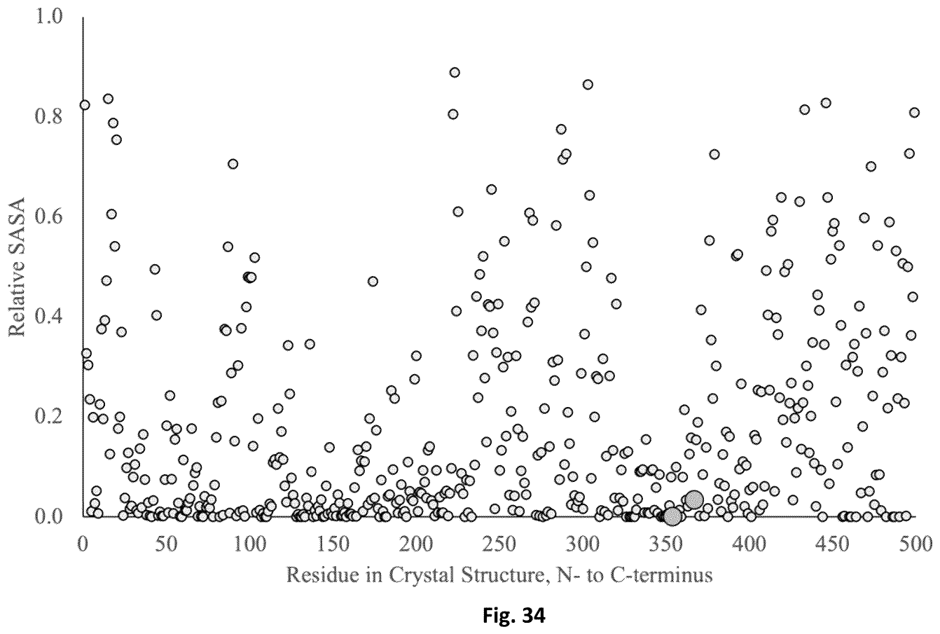

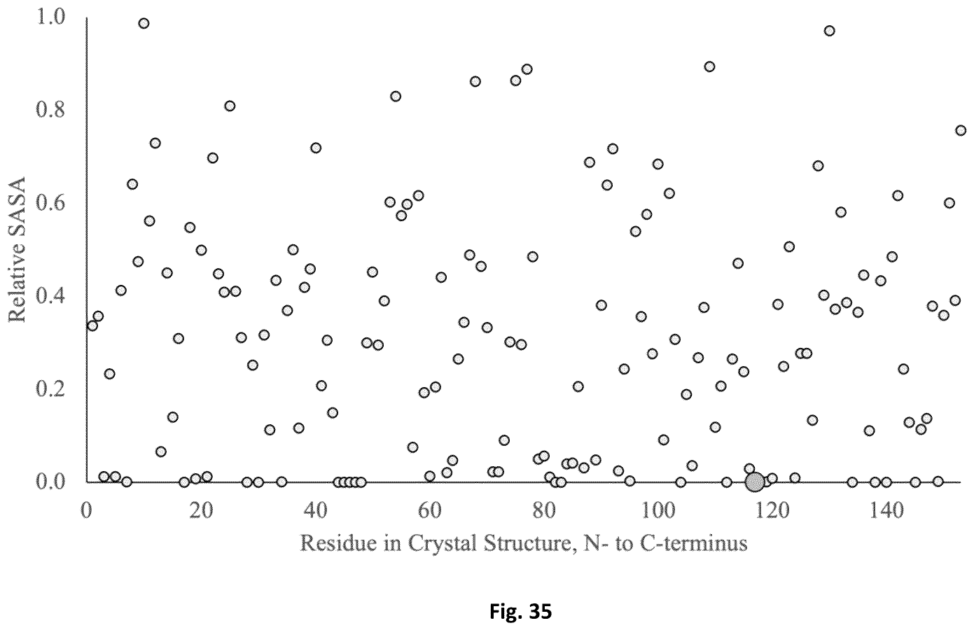

Using the crystal structures to calculate solvent accessibility across these proteins revealed that, as with lysozyme, azidylated residues are among the most buried in all three proteins (FIGS. 31F, 34, and 35). On each protein, modification either occurred within or in direct proximity to the catalytic active site. Given that active site substrate or ligand binding may be driven in part by hydrophobicity (51-53), the major azidylation sites occurring in a tissue lysate are thus consistent with similar hydrophobic forces mediating azide:protein binding. Overall, data from modifying a complex lysate demonstrated that observable azide modification occurs within 10 Å of catalytic sites, two of the three top hits are known to bind azide noncovalently, and for these two, azidylation was detected within 10 Å of co-crystallized azide (42, 49).

Taken together, our data with both pure proteins and a complex crude lysate led to a model in which direct and covalent protein azidylation occurs on three-dimensional, hydrophobic regions that may be buried and only present in folded proteins (FIG. 31G). Crystal structures of azide bound to known targets, such as F1-ATPase, indicate, via proximity to charged groups in the ligand binding sites, that ionic interactions are involved (37). These occur between the partially positive internal N atom and negatively charged terminal phosphate of ADP, as well as interactions of the anionic terminal N atoms of azide with cationic partners, such as the amino group of lysine residues. Based on the work reported herein we would like to suggest that hydrophobic properties of the azide molecule may also need to be considered in fully understanding the noncovalent binding of azide to target proteins in cells. Furthermore, given the covalent azidylation we observe in the active sites of proteins like rubisco, that were not previously considered to be azide targets, a closer examination of azide interactions with a larger number of proteins in general should be considered. These studies could consider our model invoking potential hydrophobic interactions of the azide pi electrons with either cations or with pi electrons in aromatic amino acid side chains rather than purely ionic interactions, to explain azide's biochemical role.

From a technical point of view, in order to create a chemical linker amenable to click-based enrichment or imaging, previous to our study, it was necessary to use noncanonical azide-containing amino acids during translation, conjugation chemistry (e.g., NHS ester, maleimide, etc.) containing a secondary azide moiety, or perform diazo transfer reactions with free amino groups (14, 15, 17, 18). The method described herein enables rapid and direct attachment of azide directly to protein in physiological buffer and at physiological pH, which can then be further derivatized using click chemistry. This was demonstrated using BSA, lysozyme, and coppercatalyzed azide-alkyne cycloaddition (CuAAC). We predict that azidylation also enables copper-free strain-promoted azide-alkyne cycloaddition (SPAAC), for gentler clicking conditions, and this will be the subject of future studies.

Recent advances have been made to greatly expand the utility of covalent labeling reagents for mapping interacting surfaces and reporting on conformational changes in soluble proteins (20, 54-56), new reagents have been developed that can react with aromatic sidechains within membrane proteins (6, 32, 33), and a handful of recently developed methods are aimed at measuring proteome-wide conformational changes (54-60). With our method, azide may present a unique way to address all the above. The small size of the azide anion/free radical may allow its diffusion into hydrophobic cavities that are inaccessible to larger reagents and the ability to capture and enrich for the azidylated peptides enables facile observation of low stoichiometric events and azidylation from a complex mixture. That said, we have not yet examined how sensitive azidylation is to protein conformational changes. Since azide clearly competes with palmitic acid, a ligand that binds BSA, this supports the notion that azidylation is sensitive to conformational perturbations or simple shielding induced by ligands or substrates at their binding site. Given the enrichable nature of azidylation as shown here, we envision that azidylation will ultimately complement extant techniques for assaying the conformational proteome, and future effort will focus on scaling the reaction and enrichment methods to measure chemically or genetically induced proteome-wide conformational changes.

There are also other applications for this method and model outside of the analysis of proteins in a plant cell lysate. For example, in basic or clinical research in mammalian cells, hydrophobic mapping using the azido radical may identify new regions for pharmaceutical intervention on existing drug targets, given hydrophobic interactions between a drug and target (whether via allosteric or active site inhibition) are key features of rational drug design for medical purposes (53, 61, 62). For example, azidylation could map hydrophobicity on oncogenic protein mutants or probe the role of hydrophobic patches in human proteopathic diseases such as Alzheimer's. Although the solvent accessibility and structure have been well-studied in human proteins and their mutants, azidylation may point to new, druggable hydrophobic patches that are absent in the wildtype variant but may become apparent in mutant versions of this important class of proteins. To this end, using the azido radical to directly capture and identify hydrophobic protein microenvironments may have significant promise for therapeutic drug discovery and design.

In conclusion, our data are consistent with a model in which three-dimensional protein hydrophobic microenvironments of soluble proteins can be identified by oxidatively radicalizing the azide anion to directly azidylate amino acid side chains, which can then be captured and identified using mass spectrometry. Finally, beyond empirically mapping hydrophobicity, azide-radical mediated azidylation also presents a simple and fast means for click-based derivatization of proteins in vitro using known reagents available in any lab, as a new way to enable chemical biology and synthetic protein chemistry.

### Materials and Methods

Materials. BSA (A-7906), Lysozyme (L-6876), cupric sulfate (C-7631), sodium azide (438456), iodoacetamide (11149), EZView Red Streptavidin Affinity Gel (E5529), trichloroacetic acid (T4885), dimethyl sulfoxide (34869), palmitic acid (P0500), and Immobilon-FL PVDF transfer membrane, pore size 0.45 μm (IPFL00010) were purchased from Millipore Sigma. Sodium Ascorbate (352681000) and dithiothreitol (165680050) were purchased from Acros Organics. Urea (U15), ammonium bicarbonate (A643), acetone (A929), chloroform (BP1145), hydrogen peroxide (H323), formic acid (A117), and 0.1% formic acid in water (LS118) were purchased from Fisher Scientific. PBS mix (28372), 0.1% formic acid in acetonitrile (85174), and BCA assay kit (23227) were purchased from Thermo Scientific. Benchmark prestained ladder, Bolt 4-12% Bis-tris SDAPAGE gels, NuPage MES running buffer, Bolt transfer buffer, and iBlot gel transfer stacks were purchased from Invitrogen. Chameleon Duo Prestained protein ladder, PBS Intercept blocking buffer, and IRDye800CW streptavidin (92632230) were purchased from Li-Cor. Photocleavable biotin alkyne (1118), DADPS biotin alkyne (1331) AZDye 680 Alkyne (1514), and THPTA (1010) were purchased from Click Chemistry Tools. Typsin/Lys-C mix (V507A) was purchased from Promega Corporation. eGFP (part #4999) was purchased from BioVision Incorporated, and 1cc sep-pak cartridges (WAT023590) were purchased from Waters.

Protein Azidylation and Click Chemistry. In final reaction volumes of 1 mL, BSA (10 μM) or lysozyme (70 μM) solubilized into PBS were mixed with 10% H₂O₂, 1M sodium azide, and PBS for final concentrations of 1% H₂O₂, between 1 mM and 100 mM sodium azide, and 10 μM protein. The azidylation reaction is performed as follows: Protein and azide are added together in PBS with final volume of 900 μL and are pipetted or vortexed very gently (setting 2-3) to mix to homogeneity. 100 μL of 1% H₂O₂ is added, reaction is gently vortexed (setting 2-3) or pipetted to mix for 5 seconds, then allowed to rest at room temperature for 15 seconds. 100 μL of saturated, cold trichloroacetic acid (TCA) is added to precipitate protein, sample is vortexed to mix, and put on ice for 15 minutes. Samples were spun at full speed in a microcentrifuge for 10 minutes at 4° C., and azide-containing supernatant was pipetted off and safely disposed of. Protein pellets were broken up and suspended into 500 μL ice cold acetone and put on ice for 5 minutes. Samples were spun at full speed in a microcentrifuge for 10 minutes at 4° C., and supernatant was discarded. Acetone wash was repeated a second time, and protein pellets were air dried for 5 minutes. Pellets were resolubilized into 8M urea in 50 mM ammonium bicarbonate and then diluted to 4M urea with 50 mM ammonium bicarbonate. To perform the click reaction, the following reagents were added, in the following order, for the following final concentrations: 4 μL 100 mM tris-hydroxypropyltriazolylmethylamine (THPTA) in water, final concentration 3.5 mM, 4 μL 20 mM cupric sulfate in water, final concentration 708 UM, 4 μL 300 mM sodium ascorbate in water, final concentration 10.6 mM, and 1 uL 5 mM photocleavable biotin alkyne in dimethyl sulfoxide, final concentration 44 μM. Reagents were pipetted to mix to homogeneity and reacted at room temperature for 20 minutes in the dark. From this reaction, 15 μL of the lysozyme reaction were taken for SDS-PAGE analysis and 0.5 μL of the BSA reaction were taken for SDS-Page analysis, below. The remainder of each was used for digestion and further processing, below.

SDS-PAGE and Streptavidin Blotting Analysis. Samples for SDS-PAGE analysis were evenly split and concurrently run on two gels using a Novex Bolt mini gel tank. One gel was stained with Coomassie for assessing protein amount, and one was prepped with membrane transfer buffer and transferred to a membrane for 10 minutes using an Invitrogen iBlot system. Membranes was air-dried to completion, then washed with methanol, water, and blocked with PBS Intercept blocking buffer for 1 hour. 1 μL of IRDye 800CW Streptavidin was added and reacted in the cold and dark overnight. Membranes were washed with PBS-T four times, then with PBS, then allowed to air dry in the dark. Membranes were imaged using a Li-Cor Odyssey scanner and LiCor Image Studio v3.1, and Coomassie gels were imaged using an Epson V850 Pro scanner and Silverfast 8 software.

Protein Digestion and Enrichment. Click reaction volume not used for SDS-PAGE was diluted to 1M urea with 50 mM ammonium bicarbonate, and cold, saturated TCA was added to a final concentration of ˜10%. Samples were vortexed to mix, and put on ice for 15 minutes. Samples were spun at full speed in a microcentrifuge for 10 minutes at 4° C., and supernatant was pipetted off and safely disposed of. Protein pellets were broken up and suspended into 500 μL ice cold acetone and put on ice for 5 minutes. Samples were spun at full speed in a microcentrifuge for 10 minutes at 4° C., and supernatant was discarded. Pellets were air dried, resolubilized into 8M urea in 50 mM ammonium bicarbonate and diluted to 4M urea with 50 mM ammonium bicarbonate. Dithiothreitol was added to a final concentration of 2 mM and samples were reduced at 42° C. for 40 minutes. Samples were cooled to room temperature, iodoacetamide was added to a final concentration of 5 mM, and samples were alkylated at room temperature in the dark for 40 minutes. A second aliquot of DTT was added to a final concentration of 4 mM and alkylation was quenched for 5 minutes at room temperature. Samples were diluted to 1M urea with 50 mM ammonium bicarbonate, and a 1:1 mix of trypsin/lys-C was added to a ratio of 100:1 protein:protease. Samples were digested for 12 hours at 37° C., then held at 2° C. thereafter until enrichment.

For enrichment, 50 μL of EZView Red Streptavidin affinity gel slurry was used per sample. Enrichment media was equilibrated with two washes of 1 mL 50 mM ammonium bicarbonate followed by two 1 mL washes with 1M urea in 50 mM ammonium bicarbonate. For equilibration and washes, media was spun at 8.2×G for 30 seconds to pellet gel. After equilibration, resin was moved to ice and protein digests (˜400 μL) were added directly to gel pellet from 2° C. incubation post-digest. Samples were incubated with end over end mixing for 2 hours at 4° C. in the dark. Unbound fractions were saved, and enrichment media was washed twice with 1M urea in 50 mM ammonium bicarbonate and twice with 50 mM ammonium bicarbonate. 100 μL 18 MW water was added to resin, resuspended, and samples were moved to a 250 μL thin-walled clear PCR tube. Samples were laid on their side 5 cm from a 365 nM light source on an ice pack for 30 minutes to photocleave using a Stratagene UV stratalinker 1800, and supernatant was moved to a fresh 1.5 mL low-bind microcentrifuge tube. Unbound fractions were acidified with neat formic acid to 1% and cleaned up using Agilent OMIX tips according to manufacturer's protocol, and photocleaved peptide samples were acidified with neat formic acid to 1% and cleaned up with Thermo Scientific Pierce 10 μL C18 tips according to manufacturer's protocol. Samples were dried down to completion in a vacuum centrifuge following C18 cleanup.

One-Pot Click Chemistry. 100 μM BSA was solubilized into 8M urea/50 mM ammonium bicarbonate and diluted to 50 μM with 50 mM ammonium bicarbonate. 1 μL of serial sodium azide dilutions in PBS were added to 100 μL of 50 μM BSA to achieve the final concentrations used in the experiment. CuAAC click was performed as above, with 2 μL of PC biotin alkyne in DMSO used instead of 1, final concentration 88 μM. Samples were reacted as above, 0.5 μL was sampled for Coomassie gel and blot, and the remainder was processed as above for mass spectrometry analysis.

Arabidopsis growth, lysis, and membrane clarification. Arabidopsis was grown in magenta boxes as previously described (63). Plant tissue was removed from growth liquid, gently blotted dry and weighed, and homogenized in 2× weight/volume homogenization buffer (64) by grinding for 60 seconds with a benchtop homogenizer (Pro Scientific, Inc.) at 11K rpm on ice. Homogenate was filtered through 4 layers of miracloth, then spun for 10 min at 6,000×g at 4° C. in a Sorvall RC6 Plus high-speed centrifuge (Thermo Fisher Scientific) to clarify debris. Membranes were pelleted by spinning for 45 min at 65,000×g and 4° C. in an ultrahigh-speed centrifuge (Beckman L8-80M). The supernatant from this spin, the cytosol depleted of membranes, was used for further experiments.

Arabidopsis azidylation, click chemistry, and enrichment. Protein concentration was quantified via bicinchoninic assay according to manufacturer's protocol (Thermo Scientific). Reaction was performed in a 15 ml conical tube. In final reaction volume of 1.5 mL containing PBS as the background buffer to maintain pH˜7.2, 1.1 mg of protein was mixed with 400 mM sodium azide and 1% H₂O₂ as above with pure protein. Reaction was gently vortexed for 5 s, then allowed to rest for 15 s at room temperature. Here, methanol/chloroform precipitation was used instead of TCA precipitation to stop the reaction and precipitate protein. Briefly, to 1.5 mL reaction, 6 mL of methanol, 1.5 mL of chloroform, and 4.5 mL of water were added and vortexed at room temperature. Sample was spun for 10 min in a tabletop centrifuge at room temperature and 3,500×g to facilitate phase separation. Upper phase was discarded, and 4.5 mL methanol was added to disrupt protein interface and sample was spun for 10 min in a tabletop centrifuge at room temperature and 3,500×g to pellet protein. Pellet was broken up and resuspended into 1 mL 80% acetone and transferred to a 1.5 mL microcentrifuge tube. Sample was spun for 2 min at room temp and full speed in a tabletop microcentrifuge, and supernatant was discarded. Click reaction was performed as described above, but a biotin alkyne with the acid cleavable group dialkoxydiphenylsilane was used instead of a photocleavable variant. Following clicking, a second round of methanol/chloroform precipitation was performed on a 150 μL scale (all reagents scaled 10-fold down). Reduction and alkylation were identical to above, but 10 μg of trypsin/LysC was used for digestion. Equilibration and binding to enrichment resin was as described above, however, elution was performed by adding 100 μL of 10% formic acid and incubating with vortexing for 30 min. Samples were cleaned up as described above.

ANS fluorescence and digestion assay. Empty 50 mM ammonium bicarbonate (buffer), buffer+20 μg trypsin/LysC mix (protease), buffer+1.6 μM BSA, or buffer+20 μg protease+1.6 μM BSA. Samples were equilibrated to 42° C. for 30 minutes prior to taking first fluorescence measurement and digestion was performed at 42° C. At specified timepoints, three 90 μL aliquots of respective samples were added to a plate. 10 μL of 100 μM ANS in H₂O was added and samples were immediately read with a Tecan SpectraFluor Plus plate reader using the following parameters: excitation at 360 nm, emission at 465 nm, a gain of 80, 0 μs lag time and 40 s integration time, 3 flashes, and top read mode. Samples were shaken for 10 s in orbital mode in normal intensity prior to measuring, with 0 s settle time. Averages and standard deviation per three measurement set were used to generate the graph in FIG. 27C.

Palmitate competition assay. BSA was prepared as in “Protein Azidylation” section. Palmitic acid was solubilized into chloroform and 1 μL of 100× concentrated palmitic acid (or chloroform, for control) was added to 15 μM BSA in 79 μL PBS. Samples were vortexed gently to homogeneity and let sit at room temp for 10 minutes. Azidylation and TCA precipitation then proceeded as in “Protein Azidylation” section with 100 mM azide and 1% H₂O₂. For the CuAAC click reaction, all volumes and reagent concentrations were the same, but the fluorescent AZDye 680 alkyne was used instead of the previous alkyne. 0.5 μL of the reaction was loaded onto an SDS-PAGE gel. Gel was first fluorescently imaged and then Coomassie-stained and imaged.

ANS competition assay. Lysozyme reactions were prepared as above, with an added dose curve of ANS as specified in figure. Lysozyme was gently vortexed and allowed to bind to ANS for 10 min at room temp prior to performing azidylation. Protein was TCA precipitated, clicked, digested, and analyzed as described above following azidylation.

Solvent accessibility calculations. Solvent accessibility calculations were performed using the online tool provided by the Center for Informational Biology at Ochanomizu University, located at cib.cf.ocha.ac.jp/bitool/ASA/with the PBD structures listed where used.

Mass Spectrometry of pure protein samples. Photo- and acid cleaved samples were resuspended into 10 μL Optima LC/MS-grade 0.1% formic acid, and unbound samples were resuspended into 80 μL Optima LC/MSgrade 0.1% formic acid. For both sample types, a Thermo Scientific Dionex UltiMate 3000 was used to inject peptides onto a 50 cm, 2 μM, 200 Å pore size bead-containing Thermo Scientific PepMap RSLC C18 column in a Thermo Scientific EasySpray Source. Peptides were sprayed with 1900V into a Thermo Scientific Orbitrap Fusion Lumos Tribrid Mass Spectrometer. Mobile phase A was 0.1% formic acid, and mobile phase B was 80% acetonitrile/0.1% formic acid, and flow rate was 300 nL/min. Different methods of analysis and chromatography were used; 1 μL of unbound samples were injected, and 2-3 μL of photocleaved samples were injected.

Unbound samples were analyzed using the following LC gradient: background running and equilibration buffer was 2% B, a gradient from 5% B to 37.5% B over 23 minutes, followed by a fast ramp gradient from 37.5% B to 95% B for 3 minutes, flushing at 95% B for 5 minutes, and re-equilibrated to 2% B for 10 minutes. Mass spectrometry acquisition was as follows: MS1 scans were acquired in the Orbitrap Mass analyzer with a resolution of 120K, scan range of 350-1600 m/z, AGC target of 1e6, max inject time of 50 ms, and in profile mode. For MS2 acquisition, a cycle time method with Is fixed spacing between MS1 scans was used, monoisotopic peak selection was used in peptide mode, charge states of 2-4 were selected, and dynamic exclusion was used with settings of n=1 for 10 second exclusion. MS2 spectra were acquired in the linear ion trap with quadrupole isolation window set to 0.7 m/z, scan range set to auto, a fixed HCD energy of 30% for fragmentation, scan rate set to turbo, an AGC target of 3e5, a max inject time of 25 ms, and in centroid mode.

Photo- or acid cleaved samples were analyzed using the following LC gradient: background running and equilibration buffer was 2% B, a gradient from 5% B to 37.5% B over 38 minutes, followed by a fast ramp to 37.5% B to 95% B, flushing at 95% B for 5 minutes, and re-equilibrated to 2% B for 10 minutes. Mass spectrometry acquisition was as follows: MS1 scans were acquired in the Orbitrap Mass analyzer with a resolution of 120K, scan range of 350-1600 m/z, AGC target of 1e6, max inject time of 50 ms, and in profile mode. For MS2 acquisition, a cycle time method with Is fixed spacing between MS1 scans was used, monoisotopic peak selection was used in peptide mode, charge states of 2-7 were selected for fragmentation. MS2 spectra were acquired in the Orbitrap with quadrupole isolation window set to 0.7 m/z, scan range set to auto, an AGC target of 1.5e5, a max inject time of 54 ms, and in centroid mode. Either fixed HCD collision energy of 30% or, most of the time, as specified in the main text, electron transfer dissociation with supplemental HCD activation energy was used as the fragmentation technique. Charge-dependent calibration parameters were used for ETD reaction time, and MS2 spectra were acquired in centroid mode. Dynamic exclusion was not used for cleaved samples, in order to obtain more MS2 spectra for localizing modifications on peptides.

All raw data has been uploaded to and is freely available in the PRIDE proteome database with the DOI 10.6019/PXD035808.

Database Searching. Raw data was searched in Proteome Discoverer v2.4. All data was searched against a database containing the sequence of BSA and common and lab-specific contaminants (207 proteins total).

Unbound data was searched with full tryptic cleavage specified with up to 2 missed cleavages and a minimum peptide length of 6. MS1 mass tolerance was set to 10 ppm, and MS2 mass tolerance was set to 0.6 Da. B- and y-ions were considered for matching, and both oxidation and the PC biotin cleaved tag adduct (+96.04) were set as dynamic modifications on all residues. Carbamidomethylation was set as a dynamic modification on cysteine residues. A concatenated target/decoy selection strategy was used with a strict FDR of 1%.

Photo and acid-cleaved data was searched with full tryptic cleavage specified with up to 2 missed cleavage events. MS1 mass tolerance was set to 10 ppm, and MS2 mass tolerance was set to 0.1 Da. B-, y-, c-, and z-ions were considered for matching, and both oxidation and the PC biotin cleaved tag adduct (+96.04) were set as dynamic modifications on all residues except cysteine. Carbamidomethylation was set as static on cysteine residues. A concatenated target/decoy selection strategy was used with a strict FDR of 1%.

Arabidopsis samples were searched as with cleaved samples above, but a proteome database from Uniprot (27,556 sequences, exported on Jan. 22, 2019) with added contaminants was used for searching.

All the spectra mentioned and used in the main text of the paper have been manually examined for both confidence and localization, when possible. Sites that were not localized are explicitly referred to as such.

eGFP Fluorescence assay. Triplicate samples were prepared using eGFP in PBS. Per sample, 500 ng of eGFP was used, and total reaction volumes were 100 μL. All reagents except eGFP were added together, pipetted to mix, and then eGFP was added. eGFP was measured using a Tecan SpectraFluor Plus plate reader. 5 seconds of orbital shaking followed be 15 seconds of resting (as close to our benchtop conditions as possible) were performed before measuring fluorescence. Fluorescence was measured with the following settings: excitation/emission of 485 nm/535 nm, a gain of 80, 3 flashes, 0 lag time, 40 us integration time, and at room temperature. After shaking and measuring the first time, fluorescence was measure at ˜t=1.5 min without shaking and with the above conditions.

BSA Native vs. Digest Experiment. Native/intact BSA treatment was performed as specified in main methods sections. Specifically, for the native vs. digest comparison, the same reagents were used for azidylation, CuAAC clicking, reduction, alkylation, digestion, enrichment, and solid phase extraction of enriched, photocleaved fractions. In order to azidylate peptides, BSA was first digested to peptides using the protocol in main methods section. BSA digests here were desalted and concentrated using Waters 1cc sep-pak solid phase columns, after which they were dried to completion by vacuum centrifugation. Peptides were resuspended in PBS such that final concentration was 100 μM and equivalent reaction conditions as to native protein were used. Peptide azidylation was performed as specified in main text protocol section. Per replicate, following azidylation, samples were acidified to 1% final concentration of formic acid using neat formic acid, gently vortexed, and immediately run twice through a pre-equilibrated Waters 1cc sep-pak to desalt and concentrate peptide mixture, and were then dried to completion by vacuum centrifugation. Peptides were resuspended into 8M urea/50 mM ammonium bicarbonate, diluted to 4M urea, and click was performed as described in main text section. Following click, peptides were again cleaned up using Waters 1cc sep-pak solid phase columns and dried to completion by vacuum centrifugation. Peptide mixtures were resuspened into 1M urea/50 mM ammonium bicarbonate and enriched, cleaned up, and analyzed as detailed in main text protocols.

### REFERENCES

1. H. Yu, S. Ma, Y. Li, P. A. Dalby, Hot spots-making directed evolution easier. Biotechnol Adv 56, 107926 (2022).
2. L. Lins, R. Brasseur, The hydrophobic effect in protein folding. FASEB J 9, 535-540 (1995).
3. A. Sarkar, G. E. Kellogg, Hydrophobicity—shake flasks, protein folding and drug discovery. Curr Top Med Chem 10, 67-83 (2010).
4. A. Mckenzie-Coe, N. S. Montes, L. M. Jones, Hydroxyl Radical Protein Footprinting: A Mass Spectrometry-Based Structural Method for Studying the Higher Order Structure of Proteins. Chem Rev, (2021).
5. W. Huang, K. M. Ravikumar, M. R. Chance, S. Yang, Quantitative mapping of protein structure by hydroxyl radical footprinting-mediated structural mass spectrometry: a protection factor analysis. Biophys J 108, 107-115 (2015).
6. C. Guo, M. Cheng, W. Li, M. L. Gross, Diethylpyrocarbonate Footprints a Membrane Protein in Micelles. J Am Soc Mass Spectrom 32, 2636-2643 (2021).
7. P. Sivaguru, Y. Ning, X. Bi, New Strategies for the Synthesis of Aliphatic Azides. Chem Rev 121, 4253-4307 (2021).
8. F. Minisci, R. Galli, Influence of the electrophilic character on the reactivity of free radicals in solution reactivity of alkoxy, hydroxy, alkyl and azido radicals in presence of olefins. Tetrahedron Letters 3, 533-538 (1962).
9. F. Minisci, R. Galli, Reactivity of hydroxy and alkoxy radicals in presence of olefins and oxidation-reduction systems. Introduction of azido, chloro and acyloxy groups in allylic position and azido-chlorination of olefins. Tetrahedron Letters 4, 357-360 (1963).
10. A. Singh, G. W. Koroll, R. B. Cundall, Pulse radiolysis of aqueous solutions of sodium azide:reactions of azide radical with tryptophan and tyrosine. Radiation Physics and Chemistry (1977) 19, 137-146 (1982).
11. J. Butler, E. J. Land, A. J. Swallow, W. Prutz, The azide radical and its reaction with tryptophan and tyrosine. Radiation Physics and Chemistry (1977) 23, 265-270 (1984).
12. T. Georgeá Truscott, J. Edward, Role of azide concentration in pulse radiolysis studies of oxidation: 3, 4-dihydroxyphenylalanine. Journal of the Chemical Society, Faraday Transactions 87, 2939-2942 (1991).
13. A. Gatin, I. Billault, P. Duchambon, G. Van der Rest, C. Sicard-Roselli, Oxidative radicals (HO· or N3·) induce several di-tyrosine bridge isomers at the protein scale. Free Radical Biology and Medicine 162, 461-470 (2021).
14. D. M. Patterson, L. A. Nazarova, J. A. Prescher, Finding the right (bioorthogonal) chemistry. ACS Chem Biol 9, 592-605 (2014).
15. S. L. Scinto et al., Bioorthogonal chemistry. Nat Rev Methods Primers 1, (2021).
16. N. Inoue, A. Onoda, T. Hayashi, Site-Specific Modification of Proteins through NTerminal Azide Labeling and a Chelation-Assisted CuAAC Reaction. Bioconjug Chem 30, 2427-2434 (2019).
17. S. F. van Dongen et al., Single-step azide introduction in proteins via an aqueous diazo transfer. Bioconjug Chem 20, 20-23 (2009).
18. S. Schoffelen et al., Metal-free and pH-controlled introduction of azides in proteins. Chemical Science 2, 701-705 (2011).
19. G. M. West, L. Tang, M. C. Fitzgerald, Thermodynamic analysis of protein stability and ligand binding using a chemical modification- and mass spectrometry-based strategy. Analytical chemistry 80, 4175-4185 (2008).
20. N. Wiebelhaus et al., Discovery of the Xenon-Protein Interactome Using Large-Scale Measurements of Protein Folding and Stability. Journal of the American Chemical Society, (2022).
21. B. B. Minkoff et al., Plasma-Generated OH Radical Production for Analyzing ThreeDimensional Structure in Protein Therapeutics. Sci Rep 7, 12946 (2017).
22. R. G. Reed, Location of long chain fatty acid-binding sites of bovine serum albumin by affinity labeling. J Biol Chem 261, 15619-15624 (1986).
23. A. Bujacz, Structures of bovine, equine and leporine serum albumin. Acta Crystallogr D Biol Crystallogr 68, 1278-1289 (2012).
24. A. A. Bhattacharya, T. Grune, S. Curry, Crystallographic analysis reveals common modes of binding of medium and long-chain fatty acids to human serum albumin. J Mol Biol 303, 721-732 (2000).
25. P. Lijnzaad, H. J. Berendsen, P. Argos, A method for detecting hydrophobic patches on protein surfaces. Proteins 26, 192-203 (1996).
26. O. S. Nnyigide, S. G. Lee, K. Hyun, Exploring the differences and similarities between urea and thermally driven denaturation of bovine serum albumin: intermolecular forces and solvation preferences. J Mol Model 24, 75 (2018).
27. J. L. England, G. Haran, Role of solvation effects in protein denaturation: from thermodynamics to single molecules and back. Annu Rev Phys Chem 62, 257-277 (2011).
28. D. M. Togashi, A. G. Ryder, A fluorescence analysis of ANS bound to bovine serum albumin: binding properties revisited by using energy transfer. J Fluoresc 18, 519-526 (2008).
29. L. Young, R. L. Jernigan, D. G. Covell, A role for surface hydrophobicity in protein-protein recognition. Protein Sci 3, 717-729 (1994).
30. R. Patil et al., Optimized hydrophobic interactions and hydrogen bonding at the target-ligand interface leads the pathways of drug-designing. PLOS One 5, e12029 (2010).
31. R. Patil et al., Optimized hydrophobic interactions and hydrogen bonding at the target-ligand interface leads the pathways of drug-designing. PLOS One 5, e12029 (2010).
32. J. Sun et al., Nanoparticles and photochemistry for native-like transmembrane protein footprinting. Nat Commun 12, 7270 (2021).
33. J. Sun, W. Li, M. L. Gross, Advances in mass spectrometry-based footprinting of membrane proteins. Proteomics 22, e2100222 (2022).
34. X. Jiang et al., Recent applications of click chemistry in drug discovery. Expert Opin Drug Discov 14, 779-789 (2019).
35. M. Shee, N. D. P. Singh, Chemical versatility of azide radical: journey from a transient species to synthetic accessibility in organic transformations. Chem Soc Rev 51, 2255-2312 (2022). \
36. H. C. Kolb, M. G. Finn, K. B. Sharpless, Click Chemistry: Diverse Chemical Function from a Few Good Reactions. Angew Chem Int Ed Engl 40, 2004-2021 (2001).
37. M. W. Bowler, M. G. Montgomery, A. G. Leslie, J. E. Walker, How azide inhibits ATP hydrolysis by the F-ATPases. Proc Natl Acad Sci USA 103, 8646-8649 (2006).
38. C. Ota, S. I. Tanaka, K. Takano, Revisiting the Rate-Limiting Step of the ANS-Protein Binding at the Protein Surface and Inside the Hydrophobic Cavity. Molecules 26 (2021).
39 J. Martell, E. Weerapana, Applications of copper-catalyzed click chemistry in activity-based protein profiling. Molecules 19, 1378-1393 (2014).
40. P. Nicholls, The reaction of azide with catalase and their significance. Biochem J 90, 331-343 (1964).
41. Inhibition of catalase in perfused rat liver by sodium azide. Ann N Y Acad Sci 168, 348-353 (1969).
42. A structure-based mechanism for copper-zinc superoxide dismutase. Biochemistry 38, 2167-2178 (1999).
43. C. R. Beddell, C. C. Blake, S. J. Oatley, An x-ray study of the structure and binding properties of iodineinactivated lysozyme. J Mol Biol 97, 643-654 (1975).
44. R. Ceron, M. Peimbert, A. Rojo-Dominguez, H. Najera, Hen lysozyme fibrillogenesis, molten globule intermediate and effect of copper salts. J Biomol Struct Dyn 10.1080/07391102.2021.2006090, 1-12 (2021).
45. Z. Feng, Y. Li, Y. Bai, Elevated temperatures accelerate the formation of toxic amyloid fibrils of hen egg-white lysozyme. Vet Med Sci 7, 1938-1947 (2021).
46. B. Ma, F. Zhang, X. Wang, X. Zhu, Investigating the inhibitory effects of zinc ions on amyloid fibril formation of hen egg-white lysozyme. Int J Biol Macromol 98, 717-722 (2017).
47. K. Valegard, D. Hasse, I. Andersson, L. H. Gunn, Structure of Rubisco from Arabidopsis thaliana in complex with 2-carboxyarabinitol-1,5-bisphosphate. Acta Crystallogr D Struct Biol 74, 1-9 (2018).
48. B. Stec, Structural mechanism of RuBisCO activation by carbamylation of the active site lysine. Proc Natl Acad Sci USA 109, 18785-18790 (2012).
49. R. Sugadev, M. N. Ponnuswamy, K. Sekar, Structural analysis of NADPH depleted bovine liver catalase and its inhibitor complexes. Int J Biochem Mol Biol 2, 67-77 (2011).
50. J. J. Perry, D. S. Shin, E. D. Getzoff, J. A. Tainer, The structural biochemistry of the superoxide dismutases. Biochim Biophys Acta 1804, 245-262 (2010).
51. P. Setny et al., Dewetting-controlled binding of ligands to hydrophobic pockets. Phys Rev Lett 103, 187801 (2009).
52. D. K. Sriramulu, S. G. Lee, Combinatorial Effect of Ligand and Ligand-Binding Site Hydrophobicities on Binding Affinity. J Chem Inf Model 60, 1678-1684 (2020).
53. C. Arter, L. Trask, S. Ward, S. Yeoh, R. Bayliss, Structural features of the protein kinase domain and targeted binding by small molecule inhibitors. J Biol Chem 10.1016/j.jbc.2022.102247, 102247 (2022).
54. R. Ma, H. Meng, N. Wiebelhaus, M. C. Fitzgerald, Chemo-Selection Strategy for Limited Proteolysis Experiments on the Proteomic Scale. Anal Chem 90, 14039-14047 (2018).
55. J. A. Espino, C. D. King, L. M. Jones, R. A. S. Robinson, In Vivo Fast Photochemical Oxidation of Proteins Using Enhanced Multiplexing Proteomics. Anal Chem 92, 7596-7603 (2020).
56. F. Jiao et al., Two-Dimensional Fractionation Method for Proteome-Wide Cross-Linking Mass Spectrometry Analysis. Anal Chem 94, 4236-4242 (2022).
57. L. M. Jones, Mass spectrometry-based methods for structural biology on a proteome-wide scale. Biochem Soc Trans 48, 945-954 (2020).
58. V. Cappelletti et al., Dynamic 3D proteomes reveal protein functional alterations at high resolution in situ. Cell 184, 545-559 e522 (2021).
59. A. Mateus, N. Kurzawa, J. Perrin, G. Bergamini, M. M. Savitski, Drug Target Identification in Tissues by Thermal Proteome Profiling. Annu Rev Pharmacol Toxicol 62, 465-482 (2022).
60. N. Wiebelhaus et al., Discovery of the Xenon-Protein Interactome Using Large-Scale Measurements of Protein Folding and Stability. J Am Chem Soc 144, 3925-3938 (2022).
61. C. S. Leung, S. S. Leung, J. Tirado-Rives, W. L. Jorgensen, Methyl effects on protein-ligand binding. J Med Chem 55, 4489-4500 (2012).
62. L. L. Lou, J. C. Martin, Selected Thoughts on Hydrophobicity in Drug Design. Molecules 26 (2021).
63. K. G. Kline, G. A. Barrett-Wilt, M. R. Sussman, In planta changes in protein phosphorylation induced by the plant hormone abscisic acid. Proc Natl Acad Sci USA 107, 15986-15991 (2010).
64. E. L. Huttlin, A. D. Hegeman, A. C. Harms, M. R. Sussman, Comparison of full versus partial metabolic labeling for quantitative proteomics analysis in Arabidopsis thaliana. Mol Cell Proteomics 6, 860-881 (2007).

What is claimed is:

1. A method of attaching an azide moiety to a biomolecule, the method comprising contacting a biomolecule in a solution with an azide and an oxidizing agent, for a time and at a temperature wherein at least one azide moiety is covalently bonded to the biomolecule to yield an azidylated biomolecule.

2. The method of claim 1, wherein the biomolecule is a protein.

3. The method of claim 2, wherein the protein is selected from the group consisting of an intracellular protein, a membrane-bound protein, a circulating protein, and an antibody.

4. The method of claim 1, wherein the biomolecule is a nucleic acid polymer.

5. The method of claim 4, wherein the nucleic acid polymer is a DNA polymer.

6. The method of claim 4, wherein the nucleic acid polymer is a RNA polymer.

7. The method of claim 1, wherein the solution is an aqueous solution.

8. The method of claim 1, wherein the solution is a non-aqueous solution.

9. The method of claim 1, wherein the oxidizing agent comprises H₂O₂.

10. The method of claim 1, wherein the oxidizing agent consists of H₂O₂.

11. The method of claim 1, wherein the oxidizing agent comprises phenyliodosohydroxy tosylate ([hydroxy(tosyloxy)iodo]benzene, CAS No. 27126-76-7).

12. The method of claim 1, comprising contacting the biomolecule with the azide for 1 second to 1 hour, at a temperature of from 4° C. to 100° C.

13. The method of claim 1, further comprising reacting the azidylated biomolecule with a reagent comprising an alkyne.

14. The method of claim 13, wherein the reaction with the alkyne is a copper-catalyzed azide-alkyne cycloaddition (“CuAAC”) reaction.

15. The method of claim 13, wherein the reaction with the alkyne is a strain-promoted alkyne-azide cycloaddition (“SPAAC”) reaction.

16. A method of attaching a reagent comprising an alkyne to a biomolecule, the method comprising:
reacting a biomolecule in a solution with an azide and a reagent comprising an alkyne, for a time and at a temperature wherein the reagent comprising an alkyne is covalently bonded to the biomolecule via a triazole linkage.

17. The method of claim 16, wherein the biomolecule is a protein.

18. The method of claim 17, wherein the protein is selected from the group consisting of an intracellular protein, a membrane-bound protein, a circulating protein, an antibody, and a folded protein comprising aromatic residues.

19. The method of claim 17, wherein the protein comprises bovine serum albumin.

20. The method of claim 16, wherein the biomolecule is a nucleic acid polymer.

21. The method of claim 20, wherein the nucleic acid polymer is a DNA polymer.

22. The method of claim 20, wherein the nucleic acid polymer is a RNA polymer.

23. The method of claim 16, wherein the solution is an aqueous solution.

24. The method of claim 16, wherein the solution is a non-aqueous solution.

25. The method of claim 16, wherein the reaction is a CuAAC reaction.

26. The method of claim 16, wherein the reaction is a SPAAC reaction.

27. The method of claim 16, comprising conducting the reaction for 1 second to 1 hour, at a temperature of from 4° C. to 100° C.
